(19) United States
(12) Patent Application Publication
(10) Pub. No.: US 2014/0331363 A1
(43) Pub. Date: Nov. 6, 2014

(54) PLANTS WITH ALTERED GLUCURONOXYLAN METHYL TRANSFERASE ACTIVITY AND METHODS OF USE

(71) Applicants: Maria Pena, Athens, GA (US); Breeanna Urbanowicz, Athens, GA (US); Jason Backe, Athens, GA (US); Malcom A. O'Neill, Winterville, GA (US); William S. York, Watkinsville, GA (US)

(72) Inventors: Maria Pena, Athens, GA (US); Breeanna Urbanowicz, Athens, GA (US); Jason Backe, Athens, GA (US); Malcom A. O'Neill, Winterville, GA (US); William S. York, Watkinsville, GA (US)

(73) Assignee: UNIVERSITY OF GEORGIA RESEARCH FOUNDATION, INC., Athens, GA (US)

(21) Appl. No.: 14/237,696

(22) PCT Filed: Aug. 9, 2012
(86) PCT No.: PCT/US12/50166

§ 371 Date: May 7, 2014

Related U.S. Application Data
(60) Provisional application No. 61/521,655, filed on Aug. 9, 2011.

(51) Int. Cl. C12N 15/82

(52) U.S. Cl. CPC C12N 15/8246
USPC 800/298; 435/419

(57) ABSTRACT

Provided herein are plants having altered glucuronoxylan methyl transferase (GXMT) activity, including reduced GXMT activity. In one embodiment the plant is a transgenic plant. Also provided herein are methods for using such plants, including methods for processing a part of a plant to result in a pulp, methods for hydrolyzing a pulp, and methods for producing a metabolic product. Further provided herein is plant material from a plant having altered GXMT activity, and pulp from a plant having altered GXMT activity.

### CROSS-REFERENCE TO RELATED APPLICATIONS

This application claims the benefit of U.S. Provisional Application Ser. No. 61/521,655, filed Aug. 9, 2011, which is incorporated by reference herein.

### GOVERNMENT FUNDING

The present invention was made with government support under Grant No. DE-AC05-000R22725, awarded by the Department of Energy. The Government has certain rights in this invention.

### BACKGROUND

The evolution of vascular tissues with rigid secondary cell walls was a critical adaptive event in the history of land plants (Niklas, 1997, The evolutionary biology of plants (University of Chicago Press, Chicago)). These tissues are required to transport water and nutrients throughout the plant body and provide the mechanical strength to sustain the extensive upright growth needed to compete for sunlight (Niklas, 1997, The evolutionary biology of plants (Univeristy of Chicago Press, Chicago)). Secondary walls have also had an impact on human life as they are a major component of wood (Petersen, 1984, The chemical composition of wood. The chemistry of solid wood, ed Rowell R M (American Chemical Society, Washington D.C.), pp 57-126) and are a source of nutrition for livestock (Jung and Allen, 1995, J Anim Sci 73:2774-2790). Moreover, these walls account for the bulk of renewable biomass that can be converted to fuel and added-value chemicals (Carroll and Somerville, 2009, Ann Rev Plant Biol 60:165-182). Such ever-increasing demands on plants for fuel and for food has led to a renewed interest in developing crops with secondary walls engineered to improve their agronomic value (Himmel et al., 2007, Science 315:804-807). However, progress in this area is limited by our incomplete understanding of the mechanisms of cell wall biosynthesis (York and O'Neill, 2008, Curr Opin Plant Biol 11:258-265, Sandhu et al., 2009, Molecular Plant 2:840-850, Scheller and Ulvskov, 2010, Annu Rev Plant Biol 61:263-289).

Cellulose, lignin, and 4-O-methyl glucuronoxylan (GX) are the principle components present in the secondary walls of eudicotyledons (York and O'Neill, 2008, Curr Opin Plant Biol 11:258-265). These polymers interact with themselves and with each other via covalent and non-covalent bonds to form a macromolecular network that determines the biological and physical properties of the secondary wall. Advances in understanding cellulose and lignin biosynthesis (Somerville, 2006, Annu Rev Cell Dev Biol 22:53-78, Vanholme et al., 2010, Plant Physiol 153:895-905) and some of the genetic factors that regulate secondary wall formation (Wang et al., 2011, Mol Plant 5:297-303) have begun to provide insight into wall structure and assembly. Much less is known about GX synthesis and the mechanisms by which this polysaccharide interacts with cellulose and lignin to form a functional wall (York and O'Neill, 2008, Curr Opin Plant Biol 11:258-265).

In hardwoods and in mature stems of the model plant Arabidopsis thaliana, GX has a backbone composed of 1,4-linked β-D-xylosyl (Xyl) residues that are often substituted at O-2 with α-D-glucuronic acid (GlcA) or 4-O-methyl α-D-glucuronic acid (4-O-MeGlcA) and at O-2 and O-3 with acetyl groups (York and O'Neill, 2008, Curr Opin Plant Biol 11:258-265, Ebringerova et al., 2005, Adv Polym Sci 186:1-67) (FIG. 1). Arabidopsis GX has approximately one uronic acid residue for every eight Xyl residues and a GlcA to 4-O-MeGlcA ratio of 1:3 (Peña et al., 2007, Plant Cell 19:549-563). 4-O-MeGlcA has been identified in all GXs that have been isolated from vascular plants (Ebringerova et al., 2005, Adv Polym Sci 186:1-67). By contrast, the avascular moss Physcomitrella patens, which does not form lignified secondary cell walls, produces a GX that lacks O-methyl-etherified GlcA (Kulkarni et al., 2012, Glycobiology 22:439-451), suggesting that O-methylation of GXs establishes key structural features of the secondary cell walls of vascular plants.

GX synthesis requires the coordinated action of numerous enzymes including glycosyltransferases (GTs), O-acetyl transferases and O-methyl transferases (York and O'Neill, 2008, Curr Opin Plant Biol 11:258-265, Peña et al., 2007, Plant Cell 19:549-563). Genetic approaches have provided limited insight into the mechanisms of GX synthesis, as plants carrying mutations in many of the putative xylan synthesis genes have severe growth and developmental defects related to abnormal secondary wall formation (Peña et al., 2007, Plant Cell 19:549-563, Brown et al., 2005, Plant Cell 17:2281-2295, Zhong et al., 2005, Plant Cell 17:3390-3408). Nevertheless, the protein encoded by Glucuronic Acid Substitution of Xylan (GUX) 1, a Family 8 GT responsible for adding the glucuronosyl substituent onto the GX backbone has been isolated and biochemically characterized in vitro (Rennie et al., 2012, Plant Physiol Epub ahead of print, PMID 22706449). Much less is known about the other GTs involved in secondary wall GX synthesis (Scheller and Ulvskov, 2010, Annu Rev Plant Biol 61:263-289, Peña et al., 2007, Plant Cell 19:549-563, Brown et al., 2007, Plant J 52:1154-1168, Wu et al., 2009, Plant J 57:718-731, Mortimer et al., 2010, Proc Nat Acad Sci USA 107:17409-17414). No xylan O-acetyl or O-methyltransferase has been isolated nor have the genes that encode these enzymes been identified. Thus, there is a lack of information regarding the biochemical mechanisms by which O-acetyl and O-methyl substituents are added to GX and how these substituents affect the structure and function of the secondary wall.

Numerous cation-dependent plant O-methyltransferases (OMTs) have been identified and shown to catalyze the transfer of the methyl group from S-adenosyl methionine (SAM) to secondary metabolites (Ibrahim et al., 1998, Plant Mol Biol 36:1-10, Lam et al., 2007, Genome 50:1001-1013, Kopycki et al., 2008, J Mol Biol 378:154-164). Such methylation expands the chemical diversity of these low molecular weight plant metabolites, which are involved in diverse biological processes that include signaling, defense and lignin biosynthesis (Ibrahim et al., 1998, Plant Mol Biol 36:1-10, Lam et al., 2007, Genome 50:1001-1013). An early report also showed that the methyl group of SAM was also transferred to endogenous xylan in a cell-free system derived from corn cobs but the enzyme was not characterized (Kauss and Hassid, 1967, J Biol Chem 242:1680-1685).

### SUMMARY OF THE INVENTION

Secondary cell walls are the dominant component of plant lignocellulosic feedstocks. The polysaccharides in secondary cell walls include cellulose, heteroxylans (glucuronoxylans, 4-O-methyl glucuronoxylan, glucuronoarabinoxylans, and/or arabinoxylans) and glucomannans. These polysaccharides are converted in a process known as saccharification to fermentable sugars for the production of liquid fuels and other chemical feedstocks. The cost of bioconversion to these products is increased by the recalcitrance of lignocellulosic feedstocks to saccharification.

Provided herein are methods for generating and/or identifying biofuels plant lines containing heteroxylans with altered side chain structure and/or composition due to altered expression or activity of glucuronoxylan methyl transferases (GXMT) encoded by so-called domain of unknown function 579 (DUF579) genes and their orthologs. Biofuels crops can be manipulated via genetic transformation or directed breeding to produce plants that have, for instance, non-functional copies of these GXMT genes, modified expression of these GXMT genes or modified activity of the GXMT proteins. GXMT genes encode O-methyl transferases that participate in biosynthesis of heteroxylans in plants by catalyzing the transfer of methyl groups from a suitable methyl donor to O-4 of the glucuronosyl residues of the heteroxylan. Mutant gxmt-1 A. thaliana plants with no functional copies of one member of the GXMT gene family (GXMT-1) produce secondary cell walls that contain glucuronoxylan with substantially reduced levels of 4-O-methyl-glucuronic acid and much higher levels of unmethylated glucuronic acid. Lignocellulosic material from giant-1 stems also show differences in recalcitrance to enzyme-catalyzed saccharification when compared to lignocellulosic material prepared from the stems of wild-type plants. Thus, expression of GXMT genes in planta contributes to lignocellulosic recalcitrance to saccharification. Biofuels plants lacking GXMT genes or that have reduced levels of expression of GXMT will provide improved lignocellulosic feedstock for the cost-effective production of liquid biofuels and other chemical feedstocks.

Provided herein are methods for using a transgenic plant. In one embodiment, the method includes processing a part of a transgenic plant to result in a pulp, wherein the transgenic plant includes decreased GXMT activity compared to a control plant. In one embodiment, the vascular tissues of the transgenic plant include decreased GXMT activity compared to the vascular tissues of the control plant. In one embodiment, the method includes processing a part of a transgenic plant to result in a pulp, wherein the transgenic plant includes decreased expression of a coding region encoding a GXMT polypeptide compared to a control plant. In one embodiment, the expression of the GXMT polypeptide is undetectable. The processing may include a mechanical pretreatment, a chemical pretreatment, a biological pretreatment, or a combination thereof. The method may include processing the pulp with a hydrothermal pretreatment, for instance by contacting the pulp with water at a temperature between 130° C. and 180° C. for a time between at least 5 minutes and no greater than 120 minutes at a severity level between 2 and 5. The method may further include hydrolyzing the processed pulp. The method may further include contacting the processed pulp with a microbe, such as a eukaryote. In one embodiment, the part of the transgenic plant that is processed includes the stem. Also provided herein is the pulp made from the transgenic plant, including a pulp made by processing a part of the transgenic plant to result in a pulp. In one embodiment the transgenic plant may be a woody plant, such as a member of the genus Populus. In one embodiment the transgenic plant is switchgrass.

Provided herein are methods for hydrolyzing a pulp. In one embodiment, the pulp includes plant material from a transgenic plant, wherein the transgenic plant includes decreased GXMT activity compared to a control plant. In one embodiment, the vascular tissues of the transgenic plant include decreased GXMT activity compared to the vascular tissues of the control plant. In one embodiment, the pulp includes plant material from a transgenic plant, wherein the transgenic plant includes decreased expression of a coding region encoding a GXMT polypeptide compared to a control plant. In one embodiment, the expression of the GXMT polypeptide is undetectable. The hydrolyzing may include contacting the pulp with a composition including a cellulase under conditions suitable for hydrolysis. The method may further including contacting the hydrolyzed pulp with a microbe, such as a eukaryote. In one embodiment the transgenic plant may be a woody plant, such as a member of the genus Populus. In one embodiment the transgenic plant is switchgrass.

Provided herein are methods for producing a metabolic product. In one embodiment, the method includes contacting under conditions suitable for the production of a metabolic product a microbe with a composition including a pulp obtained from a transgenic plant, wherein the transgenic plant includes decreased GXMT activity compared to a control plant. In one embodiment, the vascular tissues of the transgenic plant include decreased GXMT activity compared to the vascular tissues of the control plant. In one embodiment, the method includes contacting under conditions suitable for the production of a metabolic product a microbe with a composition including a pulp obtained from a transgenic plant, wherein the transgenic plant includes decreased expression of a coding region encoding a GXMT polypeptide compared to a control plant. In one embodiment, the expression of the GXMT polypeptide is undetectable. The method may further include fermenting the pulp by, for instance, a simultaneous saccharification and fermentation. The microbe may be a eukaryote. In one embodiment the transgenic plant may be a woody plant, such as a member of the genus Populus. In one embodiment the transgenic plant is switchgrass.

The method may further include obtaining a metabolic product. In one embodiment, the metabolic product may include an alcohol, such as ethanol, butanol, ethylene glycol, or a diol. In one embodiment, the metabolic product may include a ketone, such as acetone. In one embodiment, the metabolic product may include an aldehyde, such as acetaldehyde. In one embodiment, the metabolic product may include an organic acid, such as lactic acid or acetic acid. In one embodiment, the metabolic product may include an alkane or an alkene.

Provided herein are methods for generating a transgenic plant having decreased recalcitrance compared to a plant of substantially the same genetic background grown under the same conditions. In one embodiment, the method includes transforming a plant cell with a polynucleotide to obtain a recombinant plant cell, and generating a transgenic plant from the recombinant plant cell, wherein the transgenic plant has decreased GXMT activity compared to a control plant. In one embodiment, the method includes transforming a plant cell with a polynucleotide to obtain a recombinant plant cell, and generating a transgenic plant from the recombinant plant cell, wherein the transgenic plant has decreased expression of a coding region encoding a GMXT polypeptide compared to a control plant. The transgenic plant may be a dicot plant or a monocot plant. The method may further include breeding the transgenic plant with a second plant, wherein the second plant is transgenic or non-transgenic. In one embodiment the transgenic plant may be a woody plant, such as a member of the genus Populus. In one embodiment the transgenic plant is switchgrass.

Also provided herein is a transgenic plant. In one embodiment, a transgenic plant includes decreased GXMT activity compared to a control plant, wherein the transgenic plant is not plant line SALK_{—}018081 or SALK_{—}087114. In one embodiment, includes decreased expression of a coding region encoding a GXMT polypeptide compared to a control plant, wherein the transgenic plant is not plant line SALK_{—}018081 or SALK_{—}087114. The transgenic plant may be a dicot plant or a monocot plant. In one embodiment the transgenic plant may be a woody plant, such as a member of the genus Populus. In one embodiment the transgenic plant is switchgrass. The transgenic plant may include a phenotype of decreased recalcitrance. Provided herein is a part of the transgenic plant, wherein the part is chosen from a leaf, a stem, a flower, an ovary, a fruit, a seed, and a callus. Also provided herein is a progeny of the transgenic plant. In one embodiment, the progeny is a hybrid plant. Also provided herein is a plant material from the transgenic plant, and a pulp from the transgenic plant, as well as a pulp made by processing a part of the transgenic plant to result in a pulp.

Also provided herein is a method for using a transgenic plant including exposing biomass obtained from the transgenic plant to conditions suitable for the production of a metabolic product. In one embodiment, the exposing includes contacting the biomass with a microbe, such as a eukaryote As used herein, the term “transgenic plant” refers to a plant that has been transformed to contain at least one modification to result in altered expression of a coding region. For example, a coding region in a plant may be modified to include a mutation to reduce transcription of the coding region or reduce activity of a polypeptide encoded by the coding region. Alternatively, a plant may be transformed to include a polynucleotide that interferes with expression of a coding region. For example, a plant may be modified to express an antisense RNA or a double stranded RNA that silences or reduces expression of a coding region by decreasing translation of an mRNA encoded by the coding region. In some embodiments more than one coding region may be affected. The term “transgenic plant” includes whole plant, plant parts (stems, branches, roots, leaves, fruit, etc.) or organs, plant cells, seeds, and progeny of same. A transformed plant of the current invention can be a direct transfectant, meaning that the DNA construct was introduced directly into the plant, such as through Agrobacterium, or the plant can be the progeny of a transfected plant. The second or subsequent generation plant can be produced by sexual reproduction, i.e., fertilization. Furthermore, the plant can be a gametophyte (haploid stage) or a sporophyte (diploid stage). A transgenic plant may have a phenotype that is different from a plant that has not been transformed.

As used herein, the term “wild-type” refers to a plant cell, seed, plant component, plant tissue, plant organ or whole plant that has not been genetically modified or treated in an experimental sense.

As used herein, the term “control plant” refers to a plant that is the same species as a transgenic plant, but has not been transformed with the same polynucleotide used to make the transgenic plant.

As used herein, the term “plant tissue” encompasses any portion of a plant, including plant parts (stems, branches, roots, leaves, fruit, etc.) or organs, plant cells, and seeds. Plant cells include suspension cultures, callus, embryos, meristematic regions, callus tissue, leaves, roots, shoots, gametophytes, sporophytes, pollen, seeds and microspores. Plant tissues can be grown in liquid or solid culture, or in soil or suitable media in pots, greenhouses or fields. As used herein, “plant tissue” also refers to a clone of a plant, seed, progeny, or propagule, whether generated sexually or asexually, and descendents of any of these, such as cuttings or seeds.

Unless indicated otherwise, as used herein, “altered expression of a coding region” refers to a change in the transcription of a coding region, a change in translation of an mRNA encoded by a coding region, or a change in the activity of a polypeptide encoded by the coding region.

As used herein, “transformation” refers to a process by which a polynucleotide is inserted into the genome of a plant cell. Such an insertion includes stable introduction into the plant cell and transmission to progeny. Transformation also refers to transient insertion of a polynucleotide, wherein the resulting transformant transiently expresses a polypeptide that may be encoded by the polynucleotide.

As used herein, “phenotype” refers to a distinguishing feature or characteristic of a plant which can be altered as described herein by modifying expression of at least one coding region in at least one cell of a plant. The modified expression of at least one coding region can confer a change in the phenotype of a transformed plant by modifying any one or more of a number of genetic, molecular, biochemical, physiological, morphological, or agronomic characteristics or properties of the transformed plant cell or plant as a whole. Whether a phenotype of a transgenic plant is altered is determined by comparing the transformed plant with a plant of the same species that has not been transformed with the same polynucleotide (a “control plant”).

As used herein, “mutation” as used herein refers to a modification of the natural nucleotide sequence of a coding region or an operably linked regulatory region in such a way that the polypeptide encoded by the modified nucleic acid is altered structurally and/or functionally, or the coding region is expressed at a decreased level. Mutations may include, but are not limited to, mutations in a 5′ or 3′ untranslated region (UTR) or an exon, and such mutations may be a deletion, insertion, or point mutation to result in, for instance, a codon encoding a different amino acid or a stop to translation.

As used herein, a “target coding region” and “target coding sequence” refer to a specific coding region whose expression is inhibited by a polynucleotide described herein. As used herein, a “target mRNA” is an mRNA encoded by a target coding region.

As used herein, the term. “polypeptide” refers broadly to a polymer of two or more amino acids joined together by peptide bonds. The term “polypeptide” also includes molecules which contain more than one polypeptide joined by a disulfide bond, or complexes of polypeptides that are joined together, covalently or noncovalently, as multimers (e.g., dimers, tetramers). Thus, the terms peptide, oligopeptide, and protein are all included within the definition of polypeptide and these terms are used interchangeably.

As used herein, a polypeptide may be “structurally similar” to a reference polypeptide if the amino acid sequence of the polypeptide possesses a specified amount of sequence similarity and/or sequence identity compared to the reference polypeptide. Thus, a polypeptide may be “structurally similar” to a reference polypeptide if, compared to the reference polypeptide, it possesses a sufficient level of amino acid sequence identity, amino acid sequence similarity, or a combination thereof.

As used herein, the term “polynucleotide” refers to a polymeric form of nucleotides of any length, either ribonucleotides, deoxynucleotides, peptide nucleic acids, or a combination thereof, and includes both single-stranded molecules and double-stranded duplexes. A polynucleotide can be obtained directly from a natural source, or can be prepared with the aid of recombinant, enzymatic, or chemical techniques. A polynucleotide described herein may be isolated.

An “isolated” polynucleotide or polypeptide is one that has been removed from its natural environment. Polynucleotides and polypeptides that are produced by recombinant, enzymatic, or chemical techniques are considered to be isolated and purified by definition, since they were never present in a natural environment.

A “regulatory sequence” is a nucleotide sequence that regulates expression of a coding sequence to which it is operably linked. Nonlimiting examples of regulatory sequences include promoters, enhancers, transcription initiation sites, translation start sites, translation stop sites, transcription terminators, and poly(A) signals. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

The term “complementary” refers to the ability of two single stranded polynucleotides to base pair with each other, where an adenine on one polynucleotide will base pair to a thymine or uracil on a second polynucleotide and a cytosine on one polynucleotide will base pair to a guanine on a second polynucleotide.

“Hybridization” includes any process by which a strand of a nucleic acid sequence joins with a second nucleic acid sequence strand through base-pairing. Thus, strictly speaking, the term refers to the ability of a target sequence to bind to a test sequence, or vice-versa.

“Hybridization conditions” are typically classified by degree of “stringency” of the conditions under which hybridization is measured. The degree of stringency can be based, for example, on the calculated (estimated) melting temperature (Tm) of the nucleic acid sequence binding complex or probe. Calculation of Tm is known in the art (see Sambrook et al., 2001, Molecular Cloning, A Laboratory Manual, 3d Ed., Cold Spring Harbor Laboratory Press, Cold Spring Harbor, N.Y.). For example, “maximum stringency” typically occurs at about Tm −5° C. (5° below the Tm of the probe); “high stringency” at about 5-10° C. below the Tm; “intermediate stringency” at about 10-20° C. below the Tm of the probe; and “low stringency” at about 20-25° C. below the Tm. In general, hybridization conditions are carried out under high ionic strength conditions, for example, using 6×SSC or 6×SSPE. Under high stringency conditions, hybridization is followed by two washes with low salt solution, for example 0.5×SSC, at the calculated temperature. Under medium stringency conditions, hybridization is followed by two washes with medium salt solution, for example 2×SSC. Under low stringency conditions, hybridization is followed by two washes with high salt solution, for example 6×SSC. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, one will typically desire to employ relatively stringent conditions to form the hybrids, e.g., one will select relatively high temperature conditions. Hybridization conditions, including moderate stringency and high stringency, are provided in Sambrook et al., Molecular Cloning: A Laboratory Manual, Second Edition, Cold Spring Harbor Press (1989); Sambrook et al., Molecular Cloning, A Laboratory Manual, 3d Ed., Cold Spring Harbor Laboratory Press, Cold Spring Harbor, N.Y. (2001).

As used herein, “recalcitrance” refers to the natural resistance of plant cell walls to microbial and/or enzymatic deconstruction.

Conditions that are “suitable” for an event to occur, such as methylation of glucuronoxylan, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.). For any method disclosed herein that includes discrete steps, the steps may be conducted in any feasible order. And, as appropriate, any combination of two or more steps may be conducted simultaneously.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Schematic structure of GX. Arabidopsis GX has a linear backbone of 1,4-linked β-D-Xyl residues. Approximately one in eight of these residues are substituted at O-2 with a single α-D-GlcA residue, which is usually modified by transfer of a methyl substituent to O-4 (arrow), forming a 4-O-methyl-α-D-GlcA (i.e., 4-O-MeGlcA) residue. The distinct reducing-end sequence shown is present in Arabidopsis, softwood and hardwood GXs (York and O'Neill, 2008, Curr Opin Plant Biol 11:258-265).

FIG. 2. O-methylation of GlcA is reduced in the GX produced by GXMT1 mutants. (A) Partial 600-MHz ¹H NMR spectra of the oligosaccharides generated by endoxylanase treatment of the 1 N KOH-soluble GX from wild-type, gxmt1-1, gxmt1-2 and irx10 stem cell walls. U1 is H1 of α-D-GlcpA, M1 is H1 of 4-O-methyl α-D-GlcpA; U5 is H5 of α-D-GlcpA, M5 is H5 of 4-O-methyl α-D-GlcpA, G is H1 of α-D-GalpA, R is H1 of α-L-Rhap and X is H1 of β-D-Xylp linked to Rha. The extent of GlcA methylation was obtained by integration of U1 and M1. (B) Indirect immunofluorescence microscopy of (C) CBM2b1-2 CBMs and (D) CBM35 binding to transverse sections of wild-type, gxmt1-1, and irx10 stems. Scale bars 10 μm.

FIG. 3. Heterologously expressed GXMT1 catalyzes 4-O-methylation of GX in vitro and is located in the Golgi. (A)¹H NMR spectra of the oligosaccharides generated by endoxylanase treatment of the products formed when gxmt1-1 GX was incubated with GXMT1 and SAM. GlcA O-methylation was quantified by integration of signals labeled U1 and M1 (see FIG. 2). Kinetics of methyl transfer to (B) oligomeric (GXO) or (C) polymeric (GXP) gxmt1-1 GX as determined by measuring the amounts of SAH formed upon transfer of the methyl group from SAM in the presence of GXMT1 (340 pmol). Error bars are ±SD, n=3. Kinetic constants Kₘ (mM) and Vₘₐₓ (pmol SAH min⁻¹) were calculated by fitting the initial velocities (V₀, pmol SAH min⁻¹) as a function of the acceptor substrate concentration [GXO] or [GXP] (mM) to the Michaelis-Menten equation using nonlinear curve fitting (inset). (D) Subcellular localization of transiently expressed GXMT1-YFP in N. benthamiana epidermal cells observed by confocal laser-scanning microscopy. Co-expression of CFP-tagged Golgi apparatus marker (GmMan1-CFP, G-ck; left panel) and GXMT1-YFP (middle panel) shows GXMT1-YFP is co-localized with the Golgi marker in the merged image (right panel) (Scale Bar, 20 μm).

FIG. 4. Hydrothermal pretreatment releases more xylose from gxmt1-1 biomass than from wild-type biomass. (A) Glucan and xylan contents of Arabidopsis wild-type and gxmt1-1 stem biomass. (B) Total xylose (monomer plus oligomers) released during hydrothermal pretreatment at 180° C. for the specified times (min). (C) Glucose, xylose and total glucose plus xylose released by cellulase and xylanase (150 mg protein/g structural sugars in biomass) after hydrothermal pretreatment (180° C. for 11.1 min). Error bars are standard deviation n=3. HSQC spectra of the lignin-enriched material from wild-type (D) and gxmt1-1 (E) stems reveal subtle structural differences. HSQC crosspeak assignments are annotated using the nomenclature of Kim and Ralph (Kim and Ralph, 2010, Org Biomol Chem 8:576-591). Resonance assignments: A, various monolignols connected by β-O4 linkages; B, monolignols connected by phenylcoumaran linkages; G, guaiacyl residues; S, syringyl residues; H, hydroxyphenyl residues; OMe, phenolic methoxyl groups. Specific atom assignments are indicated by subscript numbers or Greek letters.

FIG. 5. Identification of Arabidopsis GXMT1 T-DNA insertion alleles. (A) Maximum likelihood phylogenetic tree of full length DUF579 family protein sequences from Arabidopsis thaliana (At), Physcomitrella patens (Pp) and Populus trichocarpa (Poptr). Amino acid sequences were aligned using ClustalW2. The tree was generated and bootstrap analysis was performed using SeaView 3.3. The two major clades, denoted Clade I and Clade II by (Brown et al., 2011, Plant J 66:401-413), are indicated. (B) Gene model of AtGXMT1 (At1g33800) showing the location of the T-DNA insertions The positions of the T-DNA insertion sites in are indicated by triangles. Exons are rectangles, with translated regions in grey and untranslated regions in black. The thick arrows (P1/P2) indicate the primer positions used for RT-PCR. C. RT-PCR detection of GXMT1 in wild-type (WT), gxmt1-1 and gxmt1-2 stem tissue. ACTIN2 expression was used as the control.

FIG. 6. Features of AtGXMT1. (A) Schematic representation of AtGXMT1. The DUF579 (residues 93-289) is shown in blue. Analysis of the GXMT1 sequence by the SVMtm Transmembrane Domain Predictor (Yuan et al., 2004, J Comp Chem 25:632-636), suggests it has a single transmembrane spanning domain located from amino acids 13-31, shown as “TMD.” The position of the predicted SAM binding motif (amino acids 113-117) is marked “SAM” and residues 204-209, marked “204-209,” are highly conserved in cation dependent OMTs from Group A1 (Fauman et al., 1999, Structure and evolution of AdoMet-dependent methyltransferases. In X. Cheng, R. M. Blumenthal (eds.) AdoMet-dependent methyltransferases: structures and functions. (World Scientific, pp. 1-38), Lam et al., 2007, Genome 50:1001-1013) and are predicted to play a role in SAM and metal binding. (B) One-to-one threading alignment of AtGXMT1 and amino acids 46-192 of Medicago sativa caffeoyl coenzyme A 3-O-methyltransferase (MsCCoAOMT, AAC28973.1), a well characterized cation dependent OMT from Group A1, generated using the Phyre2 multi-template modeling server (Kelley and Sternberg, 2009, Nature Protocols 4:363-371). Residues of MsCCoAOMT experimentally determined by Ferrer et al., to be involved in divalent metal coordination (Thr-63, Glu-67, Asp-163, Asp-189 and Asn-190) are indicated by the first, second, eighth, tenth, and eleventh triangles and those involved in binding SAM/SAH (Gle-85, Gly-87 Ser-93, Asp-111, Ala-140 and Asp-165) are designated by the third, fourth, fifth, sixth, seventh, and ninth triangles (Ferrer et al., 2005, Plant Physiol 137:1009-1017). Despite the low level of shared sequence identity, many of the functional residues are conserved in GXMT1. (C). Multiple sequence alignment of the deduced amino acid sequences of Arabidopsis DUF579 containing proteins performed with ClustalW2 (Goujon et al., 2010, Nucleic Acids Res 38:W695-W699) using the default settings and visualized in Vector NTI AlignX (Invitrogen). AtGXMT1 (At1g33800) is indicated in bold and members of the two major clades shown in FIG. 5 are indicated by brackets labeled with roman numerals. Putative functional residues in GXMT1 inferred from (B) are designated by the first, second, sixth, and seventh (metal coordination) and the third, fourth, and fifth (SAH/SAM) triangles, and are highly conserved among the DUF579 sequences. The alignment shading scheme (B, C) represents amino acid identity, conservation, and blocks of similarity. AT1G33800 and AtGXMT1, SEQ ID NO:22; AT1G09610 (GXMT2), SEQ ID NO:21; AT4G09990 (GXMT3), SEQ ID NO:25; AT1G71690, SEQ ID NO:26; AT1G27930, SEQ ID NO:28; AT3G50220, SEQ ID NO:29; AT5G67210, SEQ ID NO:30; AT1G67330, SEQ ID NO:31; AT2G15440, SEQ ID NO:32; AT4G24910, SEQ ID NO:33; MaCCoAOMT, SEQ ID NO:34.

FIG. 7. Methyl etherification of Fuc and Xyl in the pectic polysaccharide rhamnogalacturonan II is not affected gxmt1-1. Pectin fragments were obtained from wild-type and gxmt1-1 stems by treatment of AIR with a combination of pectin methyl esterase and endopolygalacturase The neutral glycosyl residue compositions of the fragments was determined by analysis of their alditol acetate derivatives. Methyl sugars were identified by GC-MS and quantified by GC-FID. Error bars represent the standard deviation of three analyses.

FIG. 8. Transverse sections of eight week-old Arabidopsis stems stained with Toludine blue. (A) Toluidine blue stained sections of wild-type (WT), gxmt1-1 and irx10. (B) Expanded view of Col WT stem cross section with cell types indicated: epidermis (ep), cortical parenchyma (cp), inter-fascicular fibers (if), phloem (ph), xylem (xy), vascular bundle (vb) and pith parenchyma (pp). Scale bars 10 uM.

FIG. 9. GXMT1 promoter GUS histochemical analysis of transverse sections of eight week-old Arabidopsis stems. Strong pGXMT1::GUS activity is observed the vascular bundles and to a lesser degree in fiber cells. (A) Upper stem. (B) Mid stem. (C) Lower stem. (D) Expanded view of lower stem cross section with cell types indicated: epidermis (ep), cortical parenchyma (cp), inter-fascicular fibers (if), phloem (ph), xylem (xy), vascular bundle (vb) and pith parenchyma (pp). Scale bars 10 μm. The expression pattern shown is representative of identical analyses performed on several independent transgenic plants expressing the GUS reporter gene driven by the putative GXMT1 promoter sequence.

FIG. 10. Cobalt is required for GXMT1 activity in vitro. (A) Purification of GST and GST-GXMT1, 1, Crude lysate; 2, column flow through; 3, eluted protein; L, Benchmark Protein ladder (Invitrogen). (B) Methyltransferase reactions (B, C) were performed for 180 min in HEPES-HCl, pH 7.5, containing GXMT1 (3.4 μM), gxmt1-1 xylan (2.2 mg/ml) and SAMe-PTS (15 μM). The transfer of a methyl group from SAM to GlcA results in the formation of S-adenosyl homocysteine (SAH). The amounts of SAH formed were determined by LC-ESI-MS (Salyan et al., 2006, Anal Biochem 349:112-117). (B) The effect on GXMT activity of different divalent metals. Protein and buffer solutions were treated with Chelex-100 resin to remove divalent cations prior to initiating the methylation reaction. Metal salts or EDTA were used at a concentration of 1 mM. (D) Temperature dependence of GXMT1 activity in the presence of 1 mM CoCl₂. Reaction mixtures were allowed to equilibrate to the specified temperature for 30 min and then the methylation reaction was initiated by the addition of SAMe (15 mM).

FIG. 11. Heterologously expressed GXMT1 catalyzes 4-O-methylation of GlcA sidechains of glucuronoxylan oligosaccharides in vitro but not free GlcA or UDP-GlcA. The following substrates were evaluated for their ability to act as acceptor substrates for GXMT1: gxmt1-1 glucuronoxylan fragments (A) free GlcA (B) and UDP-GlcA (C) were reacted with GXMT1 (10 μM) and S-adenosyl-L-methionine (1.5 mM) in presence of CoCl₂. The concentration of available GlcA was 2.27 mM for all substrates. After 48 hours the CoCl₂ in the reactions was removed by treatment with Chelex-100 resin and the products formed were characterized by ¹H NMR spectroscopy. The intensity of the signals of methylated GlcA (M1 and M5; see FIG. 1B) is higher in the H⁺ spectrum of the gxmt1-1 glucuronoxylan fragments after the incubation with GXMT1.

FIG. 12. GXMT1 activity is inhibited by the end product of the reaction, S-adenosyl homocysteine (SAH). gxmt1-1 glucuronoxylan at a concentration of 2.27 mM of available GlcA was reacted with 10 μM GXMT1 and S-adenosyl-L-methionine alone or in combination with S-adenosyl homocysteine (SAH). After 48 hours the products formed were treated with endoxylanase and the resulting oligosaccharides were characterized by ¹H NMR spectroscopy. The extent of GlcA methylation was determined by integration of the GlcA (U) and 4-O-methyl GlcA (M) H1 signals (see FIG. 1B). Incubation of gxmt1-1 glucuronoxylan with GXMT1 and 1.5 mM of SAM increased the percentage of GlcA methylated from 17% before of the incubation to 40% after 48 hours. Only an additional 4% of the GlcA was methylated when the SAM concentration was double to 3 mM suggesting that inhibition but not SAM availability was responsible for limited GlcA methylation. The O-methyl transferase activity was reduced over 50% when the reaction also contained 1.5 mM SAH indicating that SAH has an inhibitor effect in GXMT1 activity.

FIG. 13. GXMT1-YFP expression does not overlap significantly with an ER or plasma membrane marker. Confocal analysis of CFP tagged ER (A) and plasma membrane (D) marker proteins (HDEL-CFP, ER-ck; AtPIP2A-CFP, pm-ck) demonstrated diffuse expression patterns that were largely distinct from the discrete GXMT1-expressing puncta. While GXMT1-YFP (B, E) expression is directly adjacent to the HDEL-CFP and AtPIP2A-CFP positive structures, the two patterns do not overlap significantly (C, F). Although minor regions of overlap do exist, it is not clear whether these represent true overlap, or if they are an artifact of imaging due to the highly mobile nature of the GXMT1-expressing structures. (Scale bar, 20 μM)

FIG. 14. Xylanase fragmentation of glucuronoxylan in gxmt1-1 stem sections is increased by linking xylanase Xyl10B to CBM35-Abf62A. Arabidopsis inflorescence stem sections from gxmt1-1 (A) and irx10 (B) mutant plants were treated for 2 hrs with xylanase (10 uM), xylanase coupled to CBM35 (10 uM), or buffer only. Xylan fragmentation was then estimated by indirect immunofluorescence using xylan-specific CBM2b-1-2 binding. (C) The effect of xylanase action is expressed as a percentage of CBM2b-1-2 florescence intensity measured using “Image J”, relative to the untreated controls. 10-15 individual 250 nm transverse sections prepared from each of three independent plants per line (40 sections total) were used for each treatment.

FIG. 15. O-Methylation of GlcA is reduced in the GX produced by gxmt2-1 and gxmt3-1 plants. Partial 600-MHz ¹H NMR spectra of the oligosaccharides generated by endoxylanase treatment of the 1 N KOH-soluble GX from the cell walls of inflorescence stems from wild-type, gxmt2-1 and gxmt3-1 plants. U1 is H1 of α-D-GlcpA, M1 is H1 of 4-O-methyl α-D-GlcpA. Reductions in the extent of GlcA methylation (Table 1) were determined by integration of U1 and M1.

FIG. 16. Amino acid sequences of GXMT polypeptides (SEQ ID NO:1-1-28), and polynucleotide sequences (SEQ ID NO:29-40) encoding GXMT polypeptides SEQ ID NO:1, 4, 5, 8, 13, 16, 18, 20, 25, 26, 27, and 28, respectively. 19524274_peptide|Zmays|GRMZM5G844894|GRMZM5G844894_T01, SEQ ID NO:1; 17446785_peptide|Mtruncatula|Medtr3g013170|Medtr3g013170.1, SEQ ID NO:2; 1977891_peptide|Sbicolor|Sb08g006410|Sb08g006410.1, SEQ ID NO:3; 16893260_peptide|Osativa|LOC_Os12g10320|LOC_Os12g10320.1, SEQ ID NO:4; 18218496_peptide|Ptrichocarpa|POPTR_{—}0022s00320|POPTR_{—}0022s00320.1, SEQ ID NO:5; 16257772_peptide|Gmax|Glyma04g43510|Glyma04g43510.1, SEQ ID NO:6; 19599727_peptide|Zmays|GRMZM2G024440|GRMZM2G024440_T01, SEQ ID NO:7; 19618960_peptide|Zmays|GRMZM2G073943|GRMZM2G073943_T01, SEQ ID NO:8; 1969757_peptide|Sbicolor|Sb05g007090|Sb05g007090.1, SEQ ID NO:9; 16499977_peptide|Bdistachyon|Bradi4g40400|Bradi4g40400.1, SEQ ID NO:10; 19554976_peptide|Zmays|GRMZM2G071720|GRMZM2G071720_T01, SEQ ID NO:11; 17683070_peptide|Mguttatus|mgv1a010969 m.g|mgv1a010969m, SEQ ID NO:12; 16888768_peptide|Osativa|LOC_Os11g13870|LOC_Os11g13870.1, SEQ ID NO:13; 17974814_peptide|Mesculenta|cassava4.1_{—}013076 m.g|cassava4.1_{—}013076 m, SEQ ID NO:14; 16497719_peptide|Bdistachyon|Bradi4g21240|Bradi4g21240.1, SEQ ID NO:15; 18225748_peptide|Ptrichocarpa|POPTR_{—}0004s23540|POPTR_{—}0004 s23540.1, SEQ ID NO:16; 16300957_peptide|Gmax|Glyma16g00330|Glyma16g00330.1, SEQ ID NO:17; 18218808_peptide|Ptrichocarpa|POPTR_{—}0019 s10490|POPTR_{—}0019 s10490.1, SEQ ID NO:18; 19558687_peptide|Zmays|GRMZM2G391673|GRMZM2G391673_T01, SEQ ID NO:19; 18221592_peptide|Ptrichocarpa|POPTR_{—}0013s10240|POPTR_{—}0013 s10240.1, SEQ ID NO:20; 19654540_peptide|Athaliana|AT1G09610|AT1G09610.1, SEQ ID NO:21; 19652714_peptide|Athaliana|AT1G33800|AT1G33800.1, SEQ ID NO:22; 19696589_peptide|Sitalica|Si010707 m.g|Si010707m, SEQ ID NO:23; 19699711_peptide|Sitalica|Si024727 m.g|Si024727m, SEQ ID NO:24; SwgGXMT Pavirv00020389m Peptide, SEQ ID NO:25; SwgGXMT Pavirv00012454m Peptide, SEQ ID NO:26; EucalyptusGXMT Eucgr.F02961.1 Peptide, SEQ ID NO:27; EucalyptusGXMT Eucgr.I02785.1 Peptide, SEQ ID NO:28; ZeaMays GXMT GRMZM5G844894_T01, SEQ ID NO:29; RiceGXMT LOC_Os12g10320.1, SEQ ID NO:30; PopGXMT POPTR_{—}0022 s00320, SEQ ID NO:31; ZeaMays GXMT GRMZM2G073943_T01, SEQ ID NO:32; RiceGXMT LOC_Os11g13870.1, SEQ ID NO:33; PopGXMT POPTR_{—}0004 s23540.1, SEQ ID NO:34; PopGXMT POPTR_{—}0019 s10490.1, SEQ ID NO:35; PopGXMT POPTR_{—}0013s10240.1, SEQ ID NO:36; SwgGXMT Pavirv00020389m, SEQ ID NO:37; SwgGXMT Pavirv00012454m, SEQ ID NO:38; EucalyptusGXMT Eucgr.F02961.1, SEQ ID NO:39; EucalyptusGXMT Eucgr.I02785.1, SEQ ID NO:40.

FIG. 17. An amino acid alignment of 15 GXMT polypeptides and a consensus sequence. LOC_OS12G10320.1.OSA.16893260, SEQ ID NO:4; BRADI4G40400.1.BDI.16499977, SEQ ID NO:10; GRMZM2G073943_T01.ZMA.19618960, SEQ ID NO:8; SB08G006410.1.SBI.1977891, SEQ ID NO:3; BRADI4G21240.1.BDI.16497719, SEQ ID NO:15; GRMZM5G844894_T01.ZMA.19524274, SEQ ID NO:1; LOC_OS11G13870.1.OSA.16888768, SEQ ID NO:13; AT4G09990.1.ATH.19644356, SEQ ID NO:25; AT1G33800.1.ATH.19652714, SEQ ID NO:22; POPTR_{—}0019510490.1. PTR.18218808, SEQ ID NO:18; POPTR_{—}0013 S10240.1.PTR.18221592, SEQ ID NO:20; AT1G09610.1.ATH.19654540, SEQ ID NO:21; POPTR_{—}0022 S00320.1.PTR.18218496, SEQ ID NO:5; POPTR_{—}0004S23540.1.PTR.18225748, SEQ ID NO:16; AT1G71690.1.ATH.19655931, SEQ ID NO:26; Consensus, SEQ ID NO:27.

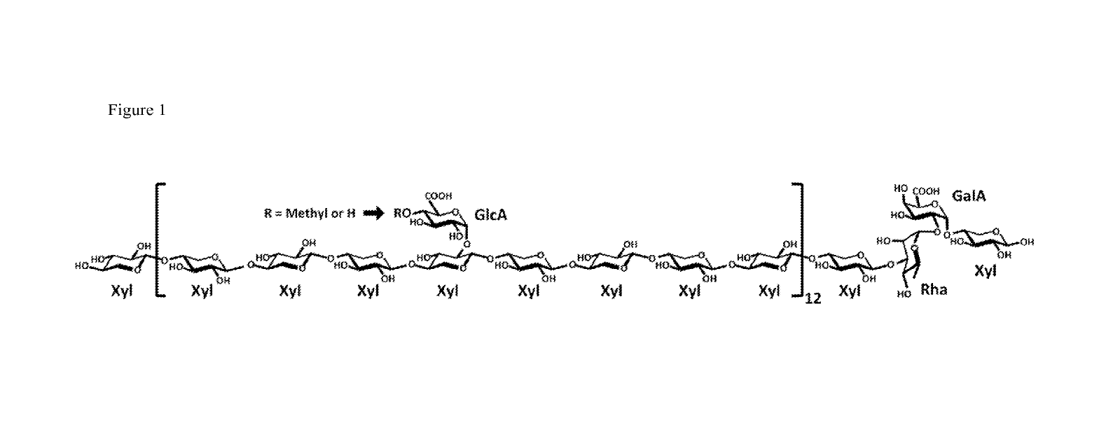

### DETAILED DESCRIPTION OF ILLUSTRATIVE EMBODIMENTS

Provided herein are cultured plant cells and plants that include alterations in expression of polypeptides having glucuronoxylan methyl transferase (GXMT) activity. The cultured plant cells and plants may be transgenic or may be natural variants. A polypeptide having GXMT activity is referred to herein as a GXMT polypeptide. The alterations in expression of a GXMT polypeptide may include, but are not limited to, a decrease in expression of an active GXMT polypeptide, expression of an inactive GXMT polypeptide, expression of a GXMT polypeptide that is altered to have decreased activity, the absence of detectable expression of a GXMT polypeptide, or a decrease in GXMT activity. More than one polypeptide may be altered in a cell or plant. In one embodiment, such modifications may be achieved by mutagenesis of a coding sequence encoding a GXMT polypeptide.

As used herein, a polypeptide having GXMT activity means a polypeptide catalyzes, under suitable conditions, the transfer of methyl groups from a suitable methyl donor to O-4 of the glucuronosyl residues of heteroxylan. Whether a polypeptide has GXMT activity may be determined by in vitro assays. In one embodiment, an in vitro assay that evaluates the candidate polypeptide's ability to transfer the methyl group from S-adenosyl methionine (SAM) to an acceptor substrate is carried out as follows (also see Example 1). Briefly, reactions containing a candidate polypeptide, SAM, and the acceptor are allowed to proceed for 48 hours, and the products formed are then analyzed. A reaction is incubated at a temperature between 19° C. and 25° C. In one embodiment, a reaction of 100 μL includes 50 mM HEPES, pH 7.5 with 3.4 μM of candidate polypeptide, 1 mM CoCl₂, 220 μg of substrate polymeric glucuronoxylan used to evaluate GXMT activity, and S-adenosyl-L-methionine sulfate p-toluenesulfonate. A reaction can be terminated by the addition of formic acid to 0.2% (v/v), and the reaction is analyzed by liquid chromatography electrospray ionization/mass spectroscopy. In one embodiment, a polypeptide having GXMT activity is divalent metal dependent, in particular Co2⁺. In one embodiment, a polypeptide having GXMT activity catalyzes the transfer of methyl groups exclusively to O-4 of GlcA in a substrate polymeric glucuronoxylan and its fragment oligosaccharides, and does not transfer methyl groups to α-D-glucuronic acid (GlcA) or UDP-GlcA at a detectable level. In one aspect, the substrate polymeric glucuronoxylan used to evaluate GXMT activity is obtained from a plant that has decreased expression of the polypeptide being tested for GXMT activity. Examples of plants that can be used as a source of substrate polymeric glucuronoxylan having a low degree of methylation include, but are not limited to, an Arabidopsis thaliana having the T-DNA insertion Salk_{—}018081 or the T-DNA insertion Salk_{—}087114 (Alonso et al., 2003, Science, 301:653-657). Seeds of A. thaliana having one of the T-DNA insertions are readily available through the Arabidopsis Biological Resource Center (ABRC) and Nottingham Arabidopsis Stock Centre (NASC) stock centers.

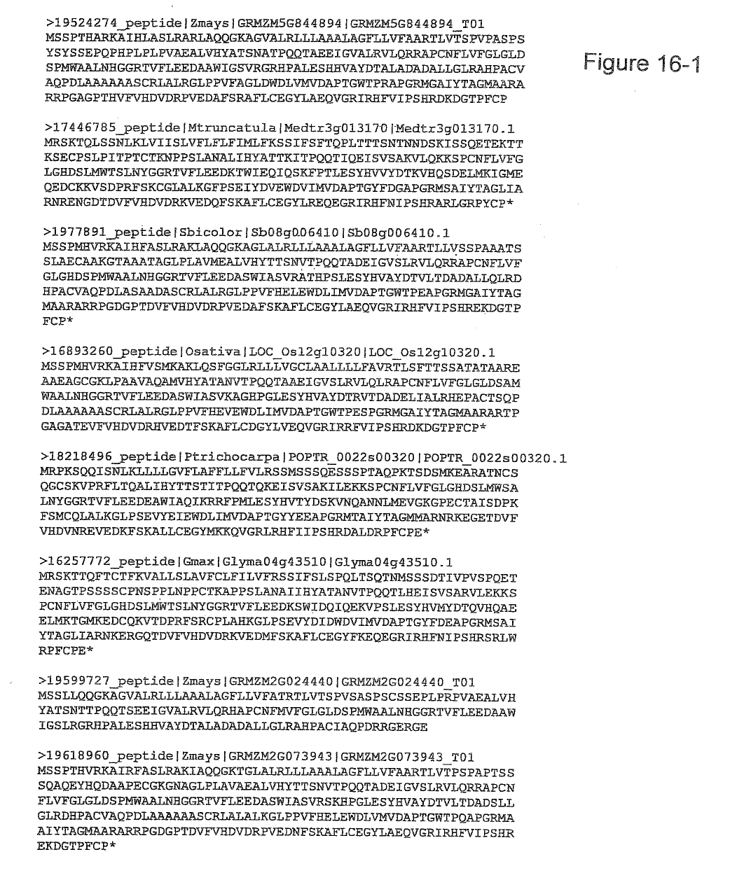

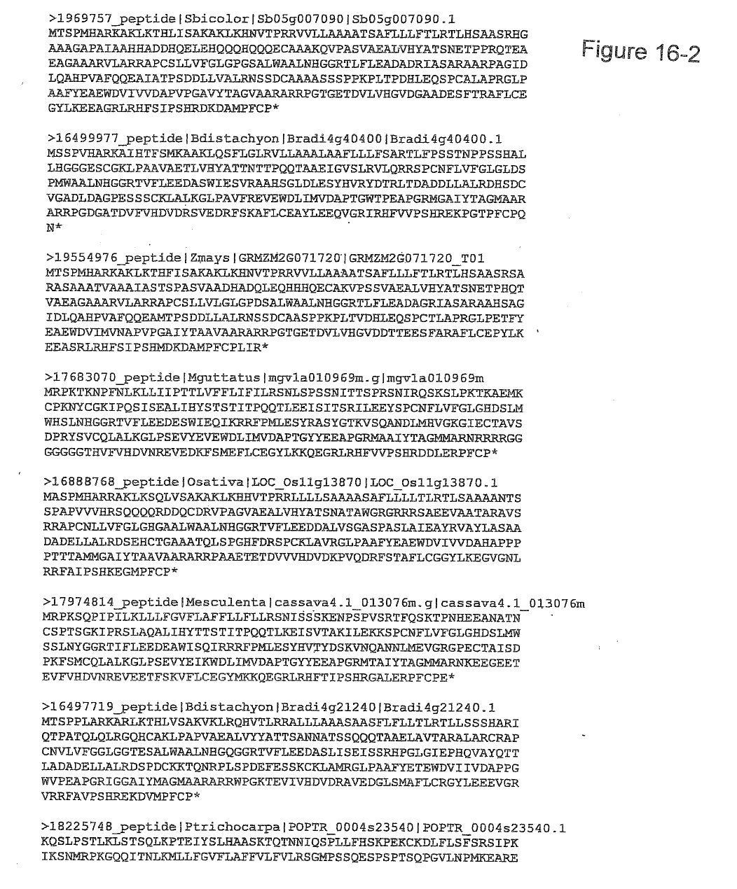

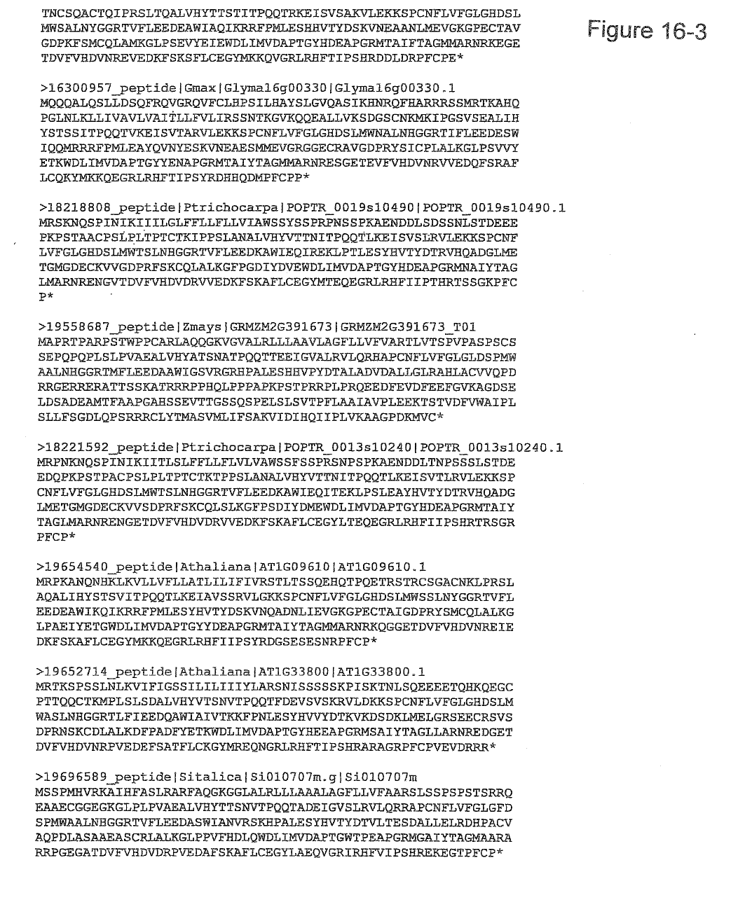

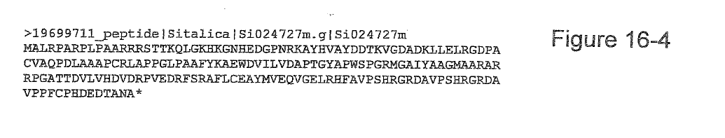

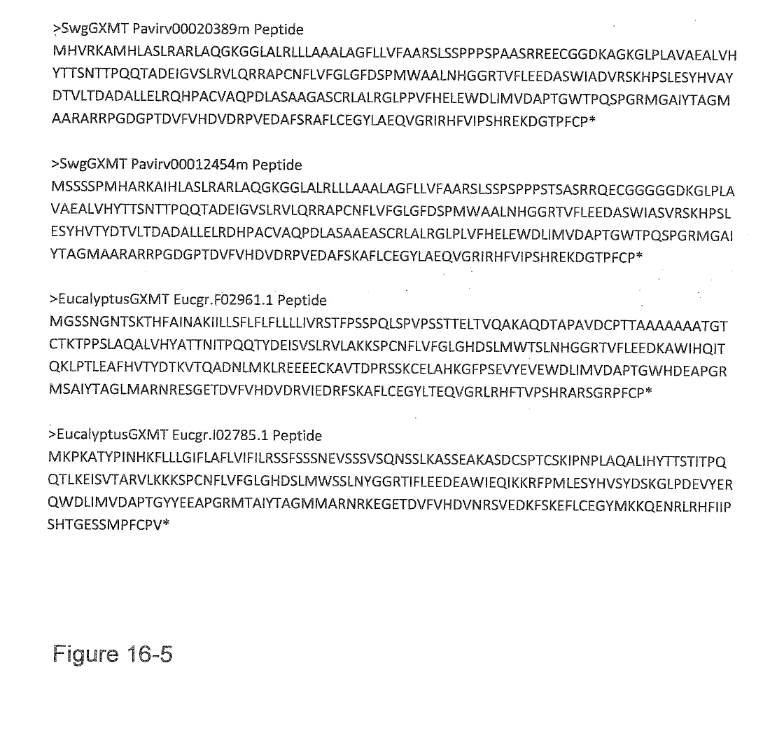

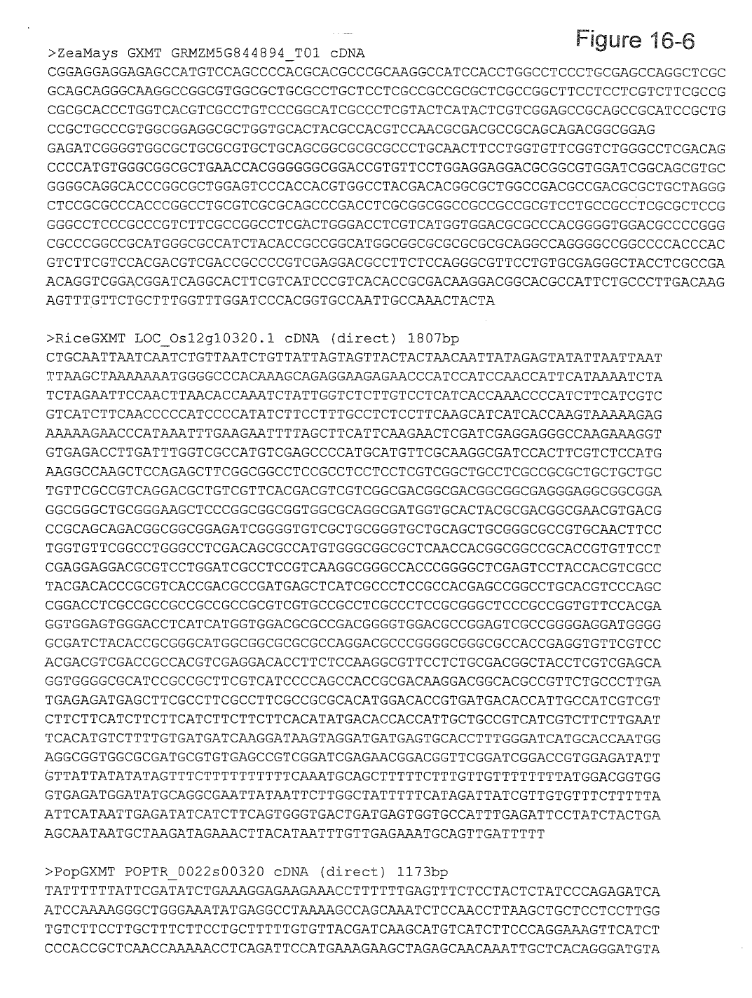

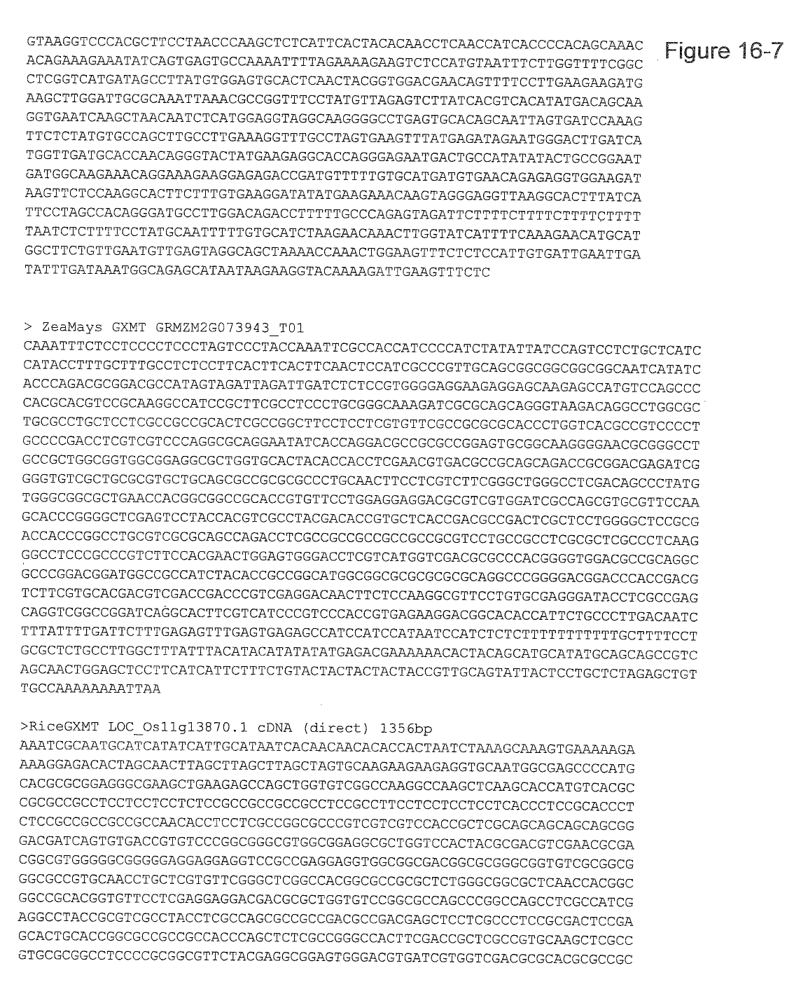

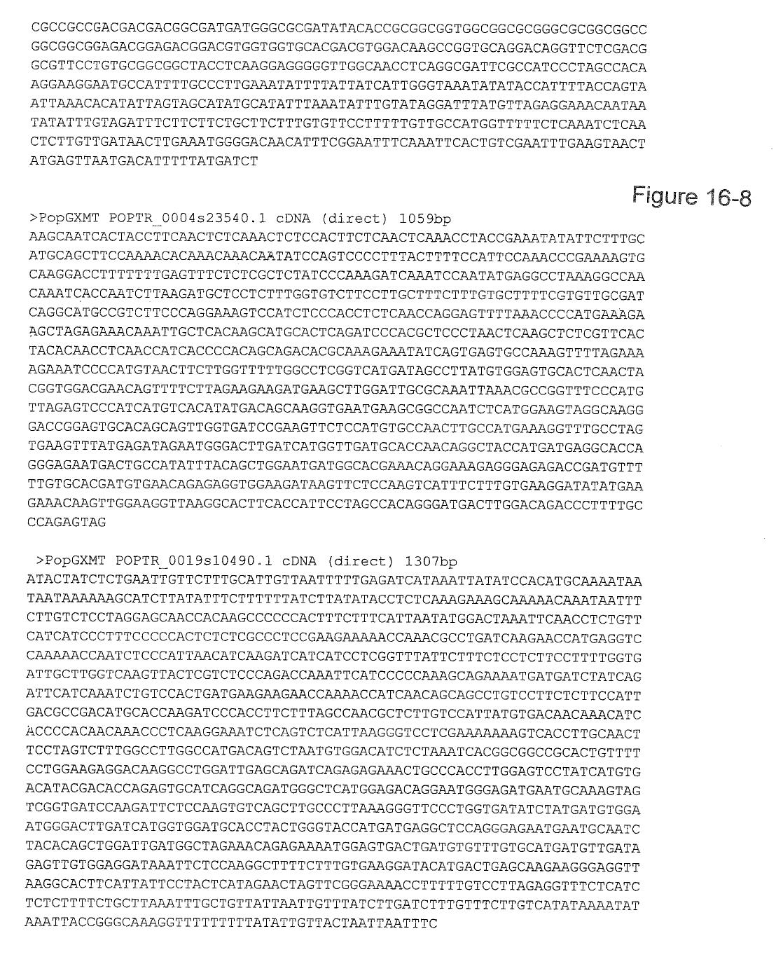

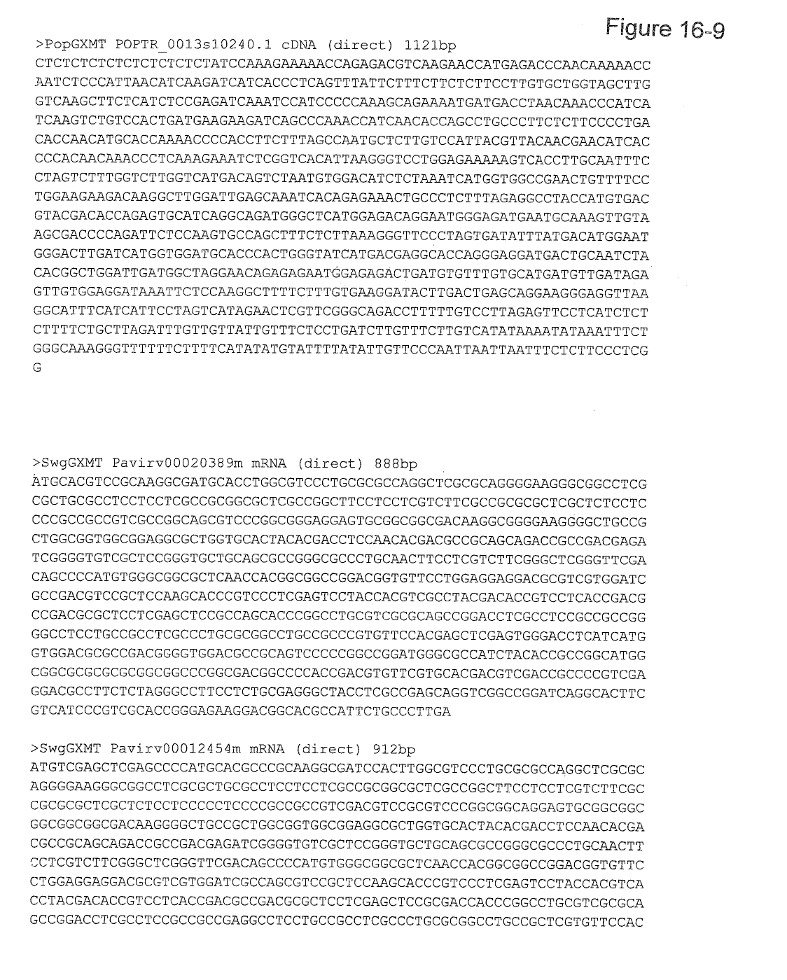

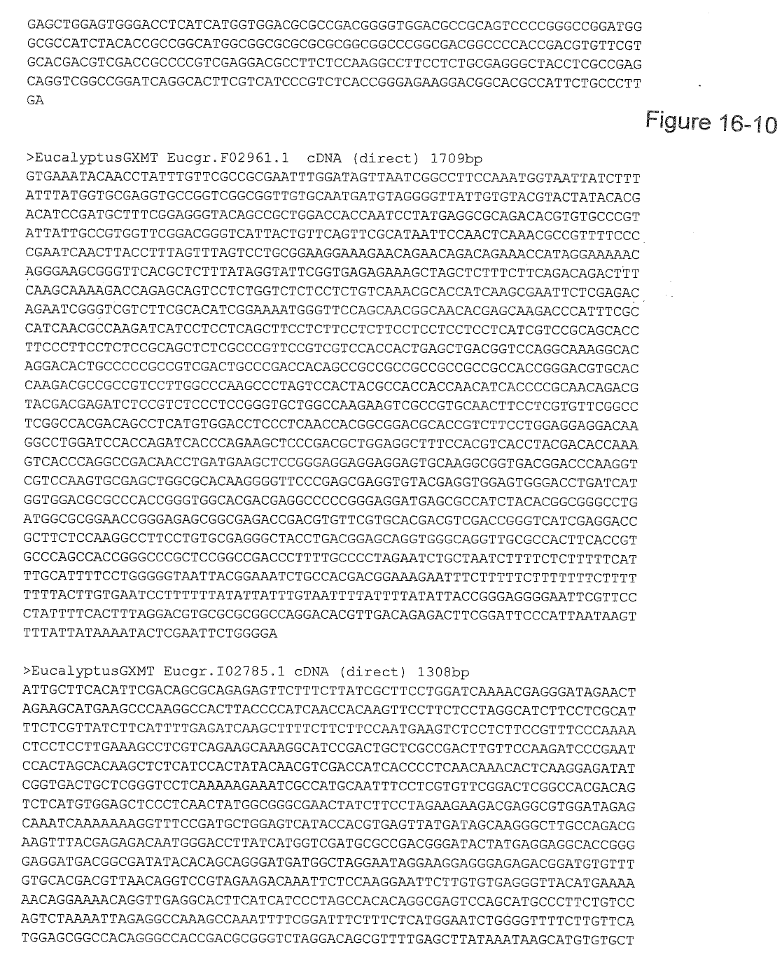

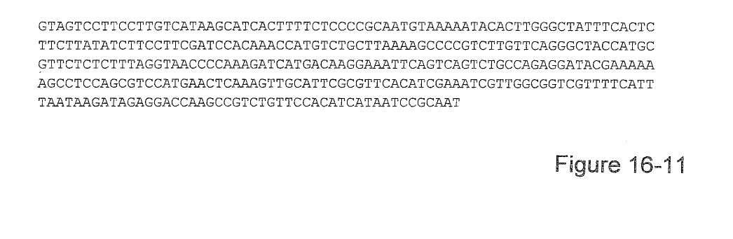

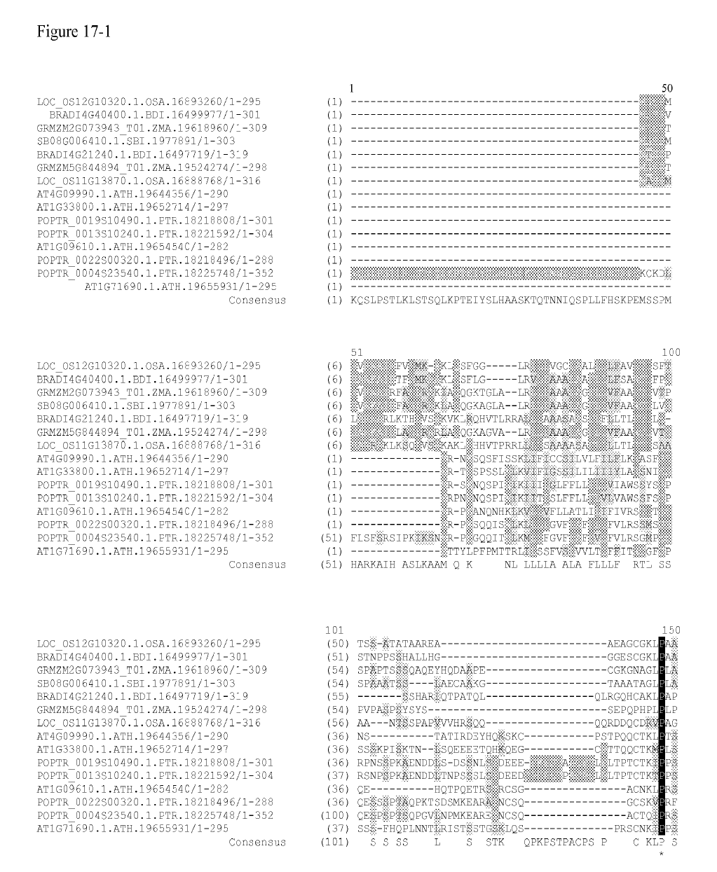

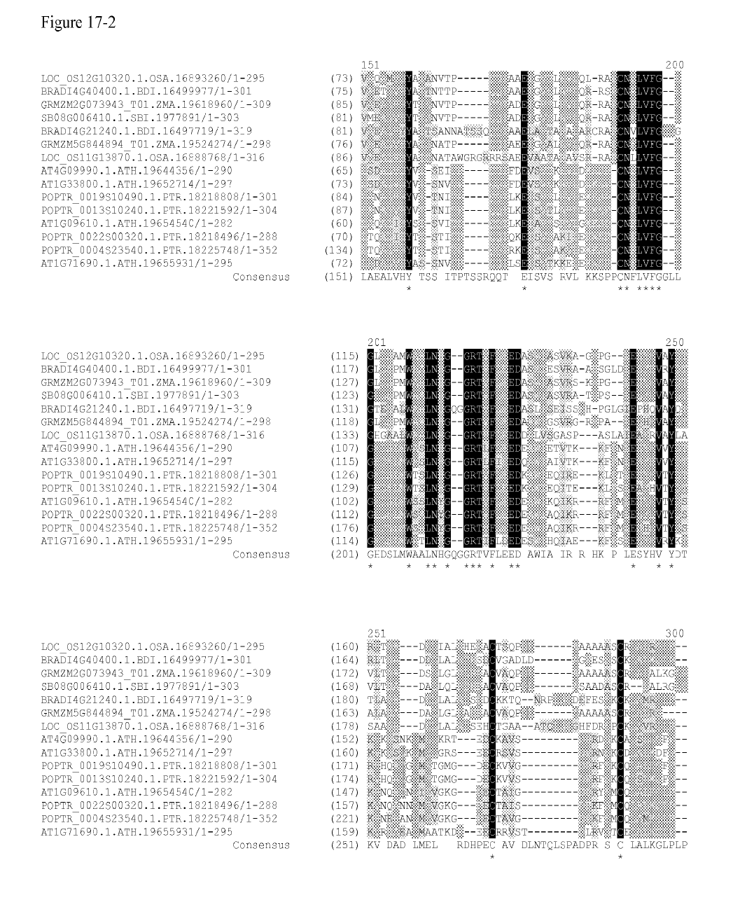

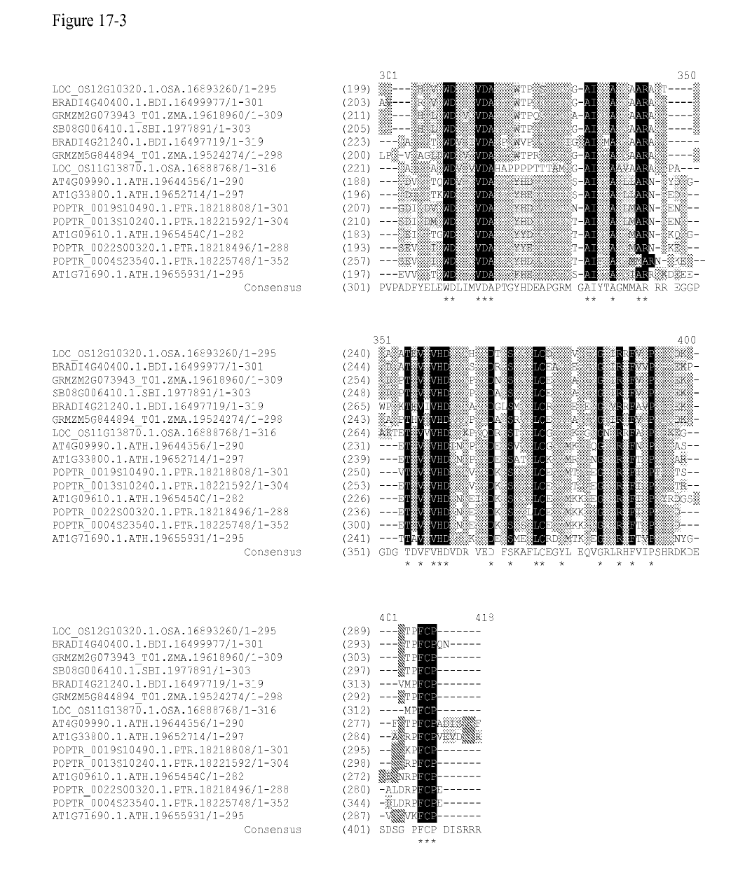

Examples of GXMT polypeptides from Z. mays, M. truncatula, S. bicolor, O. sativa, P. trichocarpa, G. max, B. distachyon, M guttatus, M esculenta, A. thaliana, S. italica, Panicum virgatum, and Eucalyptus spp. are shown in FIG. 16 (SEQ ID NOs:1-24 and 28-31). Two other GXMT polypeptides are shown in FIG. 17 (SEQ ID NO:25 and 26). Other plants have homologs, including orthologs and paralogs, of these GXMT polypeptides. Other examples of GXMT polypeptides include polypeptides having structural similarity with a reference polypeptide selected from SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:28, SEQ ID NO:29, SEQ ID NO:30, or SEQ ID NO:31.

Structural similarity of two polypeptides can be determined by aligning the residues of the two polypeptides (for example, a candidate polypeptide and any appropriate reference polypeptide described herein) to optimize the number of identical amino acids along the lengths of their sequences; gaps in either or both sequences are permitted in making the alignment in order to optimize the number of identical amino acids, although the amino acids in each sequence must nonetheless remain in their proper order. In one embodiment a reference polypeptide is a polypeptide described herein, such as any one of SEQ ID NO:1-26 or 28-31. A candidate polypeptide is the polypeptide being compared to the reference polypeptide. A candidate polypeptide can be isolated, for example, from a plant, or can be produced using recombinant techniques, or chemically or enzymatically synthesized.

Unless modified as otherwise described herein, a pair-wise comparison analysis of amino acid sequences can be carried out using the Blastp program of the blastp suite-2sequences search algorithm, as described by Tatiana et al., (FEMS Microbiol Lett, 174, 247-250 (1999)), and available on the National Center for Biotechnology Information (NCBI) website. The default values for all blastp suite-2sequences search parameters may be used, including general paramters: expect threshold=10, word size=3, short queries=on; scoring parameters: matrix=BLOSUM62, gap costs=existence:11 extension:1, compositional adjustments=conditional compositional score matrix adjustment. Alternatively, polypeptides may be compared using the BESTFIT algorithm in the GCG package (version 10.2, Madison Wis.).

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in a polypeptide of disclosed herein may be selected from other members of the class to which the amino acid belongs. For example, it is well-known in the art of protein biochemistry that an amino acid belonging to a grouping of amino acids having a particular size or characteristic (such as charge, hydrophobicity and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH2. Likewise, a polypeptide containing deletions or additions of one or more contiguous or noncontiguous amino acids that do not eliminate a GXMT activity of the polypeptide are also contemplated.

A GXMT polypeptide typically includes conserved amino acids and conserved domains. FIG. 17 depicts an amino acid alignment of 15 GXMT polypeptides and a consensus sequence. The consensus was calculated as a theoretical representative amino acid sequence in which each amino acid represents the residue seen most frequently at that same site in the aligned sequences. In FIG. 17 white letters on dark grey background refers to consensus residues derived from a block of similar residues at a given position; black letters on light grey background refers to consensus residues derived from the occurrence of greater than 50% of a single residue at a given position; and white letters on black background (also marked with an asterisk) refers to consensus residues derived from a completely conserved residue at a given position. As can be seen, 52 residues are completely conserved between each sequence, and the alignment shows multiple regions of high levels of conservation between monocots and dicots.

Thus, as used herein, reference to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:28, SEQ ID NO:29, SEQ ID NO:30, or SEQ ID NO:31 can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence similarity to the reference amino acid sequence.

Alternatively, as used herein, reference to an amino acid sequence of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:28, SEQ ID NO:29, SEQ ID NO:30, or SEQ ID NO:31 can include a polypeptide with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% amino acid sequence identity to the reference amino acid sequence.

Examples of polynucleotides encoding SEQ ID NO:1, 4, 5, 8, 13, 16, 18, 20, 28, 29, 30, and 31 are shown at SEQ ID NO:32, 33, 34, 35, 36, 37, 38, 39, 40, 41, 42, and 43, respectively. It should be understood that a polynucleotide encoding a GXMT polypeptide is not limited to a nucleotide sequence disclosed herein, but also includes the class of polynucleotides encoding the GXMT polypeptide as a result of the degeneracy of the genetic code. For example, the nucleotide sequence SEQ ID NO:32 is but one member of the class of nucleotide sequences encoding a polypeptide having the amino acid sequence SEQ ID NO:1. The class of nucleotide sequences encoding a selected polypeptide sequence is large but finite, and the nucleotide sequence of each member of the class may be readily determined by one skilled in the art by reference to the standard genetic code, wherein different nucleotide triplets (codons) are known to encode the same amino acid.

While the polynucleotide sequences described herein are listed as DNA sequences, it is understood that the complements, reverse sequences, and reverse complements of the DNA sequences can be easily deteimined by the skilled person. It is also understood that the sequences disclosed herein as DNA sequences can be converted from a DNA sequence to an RNA sequence by replacing each thymidine nucleotide with a uracil nucleotide.

Also provided herein are polynucleotide sequences having sequence similarity with SEQ ID NO:32, 33, 34, 35, 36, 37, 38, 39, 40, 41, 42, or 43 and encoding a GXMT polypeptide. Sequence similarity of two polynucleotides can be determined by aligning the residues of the two polynucleotides (for example, a candidate polynucleotide and any appropriate reference polynucleotide described herein) to optimize the number of identical nucleotides along the lengths of their sequences; gaps in either or both sequences are permitted in making the alignment in order to optimize the number of identical nucleotides, although the nucleotides in each sequence must nonetheless remain in their proper order. A reference polynucleotide may be a polynucleotide described herein. A candidate polynucleotide is the polynucleotide being compared to the reference polynucleotide. A candidate polynucleotide may be isolated, for example, from a plant, or can be produced using recombinant techniques, or chemically or enzymatically synthesized. A candidate polynucleotide may be present in the genome of a plant and predicted to encode a GXMT polypeptide.

A pair-wise comparison analysis of nucleotide sequences can be carried out using the Blastn program of the BLAST search algorithm, available through the World Wide Web, for instance at the internet site maintained by the National Center for Biotechnology Information, National Institutes of Health. Preferably, the default values for all Blastn search parameters are used. Alternatively, sequence similarity may be determined, for example, using sequence techniques such as GCG FastA (Genetics Computer Group, Madison, Wis.), MacVector 4.5 (Kodak/IBI software package) or other suitable sequencing programs or methods known in the art.

Thus, as used herein, a candidate polynucleotide useful in the methods described herein includes those with at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% nucleotide sequence identity to a reference nucleotide sequence.

Also provided herein are polynucleotides capable of hybridizing to SEQ ID N0:32, 33, 34, 35, 36, 37, 38, 39, 40, 41, 42, or 43, or a complement thereof, and encoding a GXMT polypeptide. The hybridization conditions may be medium to high stringency. A maximum stringency hybridization can be used to identify or detect identical or near-identical polynucleotide sequences, while an intermediate or low stringency hybridization can be used to identify or detect polynucleotide sequence homologs.

Provided herein are methods of using GXMT polypeptides and polynucleotides encoding GXMT polypeptides. In one embodiment, methods include altering expression of plant GXMT coding regions for purposes including, but not limited to (i) investigating function of biosynthesis of components of secondary cell walls such as glucuronoxylans and 4-O-methyl glucuronoxylan and ultimate effect on plant phenotype, (ii) investigating mechanisms of polysaccharide methylation, (iii) effecting a change in plant phenotype, and (iv) using plants having an altered phenotype.

In one embodiment, methods include altering expression of a GXMT coding region present in the genome of a plant. The plant may be a wild-type plant. In one embodiment, expression of more than one GXMT coding region present in the genome of a wild-type plant is altered. As disclosed herein, in one embodiment a wild-type plant is a woody plant, such as a member of the species Populus. In one embodiment a wild-type plant is a grass, such as a switchgrass.

Techniques which can be used in accordance with methods to alter expression of a GXMT coding region, include, but are not limited to: (i) disrupting a coding region's transcript, such as disrupting a coding region's mRNA transcript; (ii) disrupting the activity of a polypeptide encoded by a coding region, (iii) disrupting the coding region itself, (iv) modifying the timing of expression of the coding region by placing it under the control of a non-native promoter, or (v) over-expression the coding region. The use of antisense RNAs, ribozymes, double-stranded RNA interference (dsRNAi), and gene knockouts are valuable techniques for discovering the functional effects of a coding region and for generating plants with a phenotype that is different from a wild-type plant of the same species.

Antisense RNA, ribozyme, and dsRNAi technologies typically target RNA transcripts of coding regions, usually mRNA. Antisense RNA technology involves expressing in, or introducing into, a cell an RNA molecule (or RNA derivative) that is complementary to, or antisense to, sequences found in a particular mRNA in a cell. By associating with the mRNA, the antisense RNA can inhibit translation of the encoded gene product. The use of antisense technology to reduce or inhibit the expression of specific plant genes has been described, for example in European Patent Publication No. 271988, Smith et al., 1988, Nature, 334:724-726, and Smith et. al., 1990, Plant Mol. Biol., 14:369-379.

A ribozyme is an RNA that has both a catalytic domain and a sequence that is complementary to a particular mRNA. The ribozyme functions by associating with the mRNA (through the complementary domain of the ribozyme) and then cleaving the message using the catalytic domain.

RNA interference (RNAi) involves a post-transcriptional gene silencing (PTGS) regulatory process, in which the steady-state level of a specific mRNA is reduced by sequence-specific degradation of the transcribed, usually fully processed mRNA without an alteration in the rate of de novo transcription of the target gene itself. The RNAi technique is discussed, for example, in Small, 2007, Curr. Opin. Biotechnol., 18:148-153; McGinnis, 1010, Brief. Funct. Genomics, 9(2): 111-117.

Disruption of a coding region may be accomplished by T-DNA based inactivation. For instance, a T-DNA may be positioned within a polynucleotide coding region described herein, thereby disrupting expression of the encoded transcript and protein. T-DNA based inactivation can be used to introduce into a plant cell a mutation that alters expression of the coding region, e.g., decreases expression of a coding region or decreases activity of the polypeptide encoded by the coding region. For instance, mutations in a coding region and/or an operably linked regulatory region may be made by deleting, substituting, or adding a nucleotide(s). The use of T-DNA based inactiviation is discussed, for example, in Azpiroz-Leehan et al. (1997, Trends in Genetics, 13:152-156). Disruption of a coding region may also be accomplished using methods that include

transposons, homologous recombination, and the like.

Altering expression of a GXMT coding region may be accomplished by using a portion of a polynucleotide described herein. In one embodiment, a polynucleotide for altering expression of a GXMT coding region in a plant cell includes one strand, referred to herein as the sense strand, of at least 19 nucleotides, for instance, 19, 20, 21, 22, 23, 24, 25, 26, 27, 28, or 29 nucleotides (e.g., lengths useful for dsRNAi and/or antisense RNA). In one embodiment, a polynucleotide for altering expression of a GXMT coding region in a plant cell includes substantially all of a coding region, or in some cases, an entire coding region (e.g., lengths useful for T-DNA based inactivation). The sense strand is substantially identical, preferably, identical, to a target coding region or a target mRNA. As used herein, the term “identical” means the nucleotide sequence of the sense strand has the same nucleotide sequence as a portion of the target coding region or the target mRNA. As used herein, the term “substantially identical” means the sequence of the sense strand differs from the sequence of a target mRNA at least 1%, 2%, 3%, 4%, or 5% of the nucleotides, and the remaining nucleotides are identical to the sequence of the mRNA.

In one embodiment, a polynucleotide for altering expression of a GXMT coding region in a plant cell includes one strand, referred to herein as the antisense strand. The antisense strand may be at least 19 nucleotides, for instance, 19, 20, 21, 22, 23, 24, 25, 26, 27, 28, or 29 nucleotides. In one embodiment, a polynucleotide for altering expression of a GXMT coding region in a plant cell includes substantially all of a coding region, or in some cases, an entire coding region. An antisense strand is substantially complementary, preferably, complementary, to a target coding region or a target mRNA. As used herein, the team “substantially complementary” means that at least 1%, 2%, 3%, 4%, or 5% of the nucleotides of the antisense strand are not complementary to a nucleotide sequence of a target coding region or a target mRNA.

Methods are readily available to aid in the choice of a series of nucleotides from a polynucleotide described herein. For instance, algorithms are available that permit selection of nucleotides that will function as dsRNAi and antisense RNA for use in altering expression of a coding region. The selection of nucleotides that can be used to selectively target a coding region for T-DNA based inactivation may be aided by knowledge of the nucleotide sequence of the target coding region.

Polynucleotides described herein, including nucleotide sequences which are a portion of a coding region described herein, may be operably linked to a regulatory sequence. An example of a regulatory region is a promoter. A promoter is a nucleic acid, such as DNA, that binds RNA polymerase and/or other transcription regulatory elements. A promoter facilitates or controls the transcription of DNA or RNA to generate an RNA molecule from a nucleic acid molecule that is operably linked to the promoter. The RNA can encode an antisense RNA molecule or a molecule useful in RNAi. Promoters useful in the invention include constitutive promoters, inducible promoters, and/or tissue preferred promoters for expression of a polynucleotide in a particular tissue or intracellular environment, examples of which are known to one of ordinary skill in the art.

Examples of useful constitutive plant promoters include, but are not limited to, the cauliflower mosaic virus (CaMV) 35S promoter, (Odel et al., 1985, Nature, 313:810), the nopaline synthase promoter (An et al., 1988, Plant Physiol., 88:547), and the octopine synthase promoter (Fromm et al., 1989, Plant Cell 1: 977).

Examples of inducible promoters include, but are not limited to, auxin-inducible promoters (Baumann et al., 1999, Plant Cell, 11:323-334), cytokinin-inducible promoters (Guevara-Garcia, 1998, Plant Mol. Biol., 38:743-753), and gibberellin-responsive promoters (Shi et al., 1998, Plant Mol. Biol., 38:1053-1060). Additionally, promoters responsive to heat, light, wounding, pathogen resistance, and chemicals such as methyl jasmonate or salicylic acid, can be used, as can tissue or cell-type specific promoters such as xylem-specific promoters (Lu et al., 2003, Plant Growth Regulation 41:279-286).

Another example of a regulatory region is a transcription terminator. Suitable transcription terminators are known in the art and include, for instance, a stretch of 5 consecutive thymidine nucleotides.

Thus, in one embodiment a polynucleotide that is operably linked to a regulatory sequence may be in an “antisense” orientation, the transcription of which produces a polynucleotide which can form secondary structures that affect expression of a target coding region in a plant cell. In another embodiment, the polynucleotide that is operably linked to a regulatory sequence may yield one or both strands of a double-stranded RNA product that initiates RNA interference of a target coding region in a plant cell.

A polynucleotide may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the invention employs standard ligation techniques known in the art. See, e.g., Sambrook et al, Molecular Cloning: A Laboratory Manual., Cold Spring Harbor Laboratory Press (1989). A vector can provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, transposon vectors, and artificial chromosome vectors. A vector may result in integration into a cell's genomic DNA. A vector may be capable of replication in a bacterial host, for instance E. coli or Agrobacterium tumefaciens. Preferably the vector is a plasmid. In some embodiments, a polynucleotide can be present in a vector as two separate complementary polynucleotides, each of which can be expressed to yield a sense and an antisense strand of a dsRNA, or as a single polynucleotide containing a sense strand, an intervening spacer region, and an antisense strand, which can be expressed to yield an RNA polynucleotide having a sense and an antisense strand of the dsRNA.

Selection of a vector depends upon a variety of desired characteristics in the resulting construct, such as a selection marker, vector replication rate, and the like. Suitable host cells for cloning or expressing the vectors herein are prokaryotic or eukaryotic cells. Suitable eukaryotic cells include plant cells. Suitable prokaryotic cells include eubacteria, such as gram-negative organisms, for example, E. coli or A. tumefaciens.

A selection marker is useful in identifying and selecting transformed plant cells or plants. Examples of such markers include, but are not limited to, a neomycin phosphotransferase (NPTII) gene (Potrykus et al., 1985, Mol. Gen. Genet., 199:183-188), which confers kanamycin resistance, and a hygromycin B phosphotransfease (HPTII) gene (Kaster, et al, 1983, Nuc. Acid. Res. 19: 6895-6911). Cells expressing the NPTII gene can be selected using an appropriate antibiotic such as kanamycin or G418. The HPTII gene encodes a hygromycin-B 4-O-kinase that confers hygromycin B resistance. Cells expressing HPTII gene can be selected using the antibiotic of hygromycin B (Kaster, et al, 1983, Nuc. Acid. Res. 19: 6895-6911, Blochlinger and Diggelmann, 1984, Mol. Cell. Biol. 4 (12): 2929-2931). Other commonly used selectable markers include a mutant EPSP synthase gene (Hinchee et al., 1988, Bio/Technology 6:915-922), which confers glyphosate resistance; and a mutant acetolactate synthase gene (ALS), which confers imidazolinone or sulphonylurea resistance (Conner and Santino, 1985, European Patent Application 154,204).

Polynucleotides described herein can be produced in vitro or in vivo. For instance, methods for in vitro synthesis include, but are not limited to, chemical synthesis with a conventional DNA/RNA synthesizer. Commercial suppliers of synthetic polynucleotides and reagents for in vitro synthesis are well known. Methods for in vitro synthesis also include, for instance, in vitro transcription using a circular or linear expression vector in a cell free system. Expression vectors can also be used to produce a polynucleotide described herein in a cell, and the polynucleotide may then be isolated from the cell.

The invention also provides host cells having altered expression of a coding region described herein. As used herein, a host cell includes the cell into which a polynucleotide described herein was introduced, and its progeny, which may or may not include the polynucleotide. Accordingly, a host cell can be an individual cell, a cell culture, or cells that are part of an organism. The host cell can also be a portion of an embryo, endosperm, sperm or egg cell, or a fertilized egg. In one embodiment, the host cell is a plant cell.

Provided herein are transgenic plants having altered expression of a coding region. A transgenic plant may be homozygous or heterozygous for a modification that results in altered expression of a coding region.

In one embodiment, a host cell is not obtained from SALK_{—}018081 or SALK_{—}087114. In one embodiment, a transgenic plant is not plant line SALK_{—}018081 or SALK_{—}087114. In one embodiment, host cell or a transgenic plant may have a decrease in expression of an active GXMT polypeptide. In one embodiment, host cell or a transgenic plant may have expression of an inactive GXMT polypeptide. In one embodiment, host cell or a transgenic plant may have expression of a GXMT polypeptide that is altered to have decreased activity. A GXMT polypeptide that is altered to have decreased activity may be decreased by at least 10%, at least 20%, at least 30%, at least 40%, at least 50%, at least 60%, at least 70%, at least 80%, or at least 90% compared to the activity of a GXMT polypeptide in a control plant. In one embodiment, host cell or a transgenic plant may have an absence of detectable expression of a GXMT polypeptide. In one embodiment, host cell or a transgenic plant may have a decrease in GXMT activity. The GXMT activity in a host cell or a transgenic plant having decreased GXMT activity may be decreased by at least 10%, at least 20%, at least 30%, at least 40%, at least 50%, at least 60%, at least 70%, at least 80%, or at least 90% compared to the GXMT activity in a control plant.

Also provided herein are natural variants of plants. In one embodiment, a natural variant has decreased expression of a GXMT polypeptide, where the change in GXMT expression is relative to the level of expression of the GXMT polypeptide in a natural population of the same species of plant. Natural populations include natural variants, and at a low level, extreme variants (Studer et al., 2011, Proc. Nat. Acad. Sci., USA, 108:6300-6305). The level of expression of GXMT polypeptide in an extreme variant may vary from the average level of expression of the GXMT polypeptide in a natural population by at least 5%, at least 10%, at least 15%, at least 20%, or at least 25%. The average level of expression of the GXMT polypeptide in a natural population may be determined by using at least 50 randomly chosen plants of the same species as the putative extreme variant.

A plant may be an angiosperm or a gymnosperm. The polynucleotides described herein may be used to transform a variety of plants, both monocotyledonous (e.g grasses, sugar cane, corn, grains, oat, wheat, barley, rice, and the like), dicotyledonous (e.g., Arabidopsis, Brassica, tobacco, potato, tomato, peppers, melons, legumes, alfalfa, oaks, eucalyptus, maple, poplar, aspen, cottonwood, and the like).

The plants also include switchgrass (Panicum virgatum), turfgrass, sugar beet, lettuce, carrot, strawberry, cassava, sweet potato, geranium, soybean, and various types of woody plants. Woody plants include trees such as palm oak, pine, maple, fir, apple, fig, plum acacia, aspen, and willow. Woody plants also include rose and grape vines.

In one embodiment, the plants are woody plants, which are trees or shrubs whose stems live for a number of years and increase in diameter each year by the addition of woody tissue. Plants of significance in the commercial biomass industry and useful in the methods disclosed herein include members of the family Salicaceae, such as Populus spp. (e.g., Populus trichocarpa, Populus deltoides), members of the family Pinaceae, such as Pinus spp. (e.g., Pinus taeda [Loblolly Pine]), and Eucalyptus spp.

Also provided is the plant material (such as, for instance, stems, branches, roots, leaves, fruit, etc.) derived from plant described herein. In one embodiment, the plant material is present in a plant material-derived product such as lumber (including, for instance, dimensional lumber and engineered lumber). In one embodiment, a plant material-derived product is a pulp. As used herein, “pulp” refers to a mechanically, chemically and/or biologically processed wood or non-wood plant material that contains cell wall material. Cell wall material includes cell walls, cell-wall polymers and/or molecules (such as oligosaccharides) that are derived from cell wall polymers. Cell wall polymers include cellulose, hemicellulose, pectin and/or lignin. Processing to generate a pulp may increase the susceptibility of the cell wall polysaccharides to hydrolysis and fermentation. Examples of pulp include, for instance, woodchips and sawdust. Also provided is pulp derived from a plant and/or plant material described herein. The cell wall material component of a pulp may be at least 25%, at least 30%, at least 40%, at least 50%, at least 60%, at least 70%, or at least 80% cell wall material (weight cell wall material/weight total dry plant material). In one embodiment, the cell wall material component of a pulp is at least 25%, at least 30%, at least 40%, at least 50%, at least 60%, at least 70%, or at least 80% cell wall (weight cell wall/weight total dry plant material). In one embodiment, the cell wall material component of a pulp is at least 25%, at least 30%, at least 40%, at least 50%, at least 60%, at least 70%, or at least 80% cell wall polymers (weight cell wall polymers/weight total dry plant material). In one embodiment, the cell wall material component of a pulp is at least 25%, at least 30%, at least 40%, at least 50%, at least 60%, at least 70%, or at least 80% molecules derived from cell wall polymers (weight molecules derived from cell wall polymers/weight total dry plant material). In one embodiment, the cell wall material component of a pulp is no greater than 80%, no greater than 70%, no greater than 60%, no greater than 50%, no greater than 40%, or no greater than 30% molecules derived from cell wall polymers (weight molecules derived from cell wall polymers/weight total dry plant material).

Transformation of a plant with a polynucleotide described herein to result in decreased GXMT polypeptide expression may yield a phenotype including, but not limited to, changes in cell wall composition. In one embodiment the cell wall is the secondary cell wall. Changes in cell wall include changes in cell wall polysaccharide content and/or methylation of heteroxylans, such as glucuronoxylan. In one embodiment a phenotype is a decreased amount of 4-O-methyl-GlcA sidechains of glucuronoxylan. In one embodiment, a phenotype is an increase in the release of xylose during pretreatment compared to a control plant. In one embodiment, such a pretreatment includes exposure of plant biomass to a hydrothermal step. The conditions of such a hydrothermal pretreatment are described herein. In one embodiment a phenotype is reduced recalcitrance compared to a control plant. Methods for measuring recalcitrance are routine and include, but are not limited to, measuring changes in the extractability of carbohydrates, where an increase in extractability suggests a cell wall that is more easily solubilized, and thus, decreased recalcitrance. Another test for measuring changes in recalcitrance uses microbes as described in Mohnen et al. (WO 2011/130666). In one embodiment a phenotype is a change in lignin monomer composition, such as an increase in lignin methylation, compared to a control plant.

Other phenotypes present in a transgenic plant described herein may include yielding biomass with reduced recalcitrance and from which sugars can be released more efficiently for use in biofuel and biomaterial production, yielding biomass which is more easily deconstructed and allows more efficient use of wall structural polymers and components, and yielding biomass that will be less costly to refine for recovery of sugars and biomaterials.

Phenotype can be assessed by any suitable means. The biochemical characteristics of lignin, cellulose, carbohydrates and other plant extracts can be evaluated by standard analytical methods including spectrophotometry, fluorescence spectroscopy, HPLC, mass spectroscopy, molecular beam mass spectroscopy, near infrared spectroscopy, nuclear magnetic resonance spectroscopy, and tissue staining methods.

One method that can be used to evaluate the phenotype of a transgenic plant is glycome profiling. Glycome profiling gives information about the presence of carbohydrate structures in plant cell walls, including changes in the extractability of carbohydrates, such as xylose, from cell walls (Zhu et al., 2010, Mol. Plant, 3:818-833; Pattathil et al., 2010, Plant Physiol., 153:514-525), the latter providing information about larger scale changes in wall structure. Diverse plant glycan-directed monoclonal antibodies are available from, for instance, CarboSource Services (Athens, Ga.), and PlantProbes (Leeds, UK). The change in extractability may be an increase or a decrease of one or more carbohydrates in an extracted fraction compared to a control plant. In one embodiment the change is an increase of one or more carbohydrates in an extracted fraction compared to a control plant. Examples of solvents useful for evaluating the extractability of carbohydrates include, but are not limited to, oxalate, carbonate, KOH (e.g., 1M and 4M), and chlorite.

Transgenic plants described herein may be produced using routine methods. Methods for transformation and regeneration are known to the skilled person. Transformation of a plant cell with a polynucleotide described herein may be achieved by any known method for the insertion of nucleic acid sequences into a prokaryotic or eukaryotic host cell, including Agrobacterium-mediated transformation protocols, viral infection, whiskers, electroporation, microinjection, polyethylene glycol-treatment, heat shock, lipofection, particle bombardment, and chloroplast transformation.

Transformation techniques for dicotyledons are known in the art and include Agrobacterium-based techniques and techniques that do not require Agrobacterium. Non-Agrobacterium techniques involve the uptake of exogenous genetic material directly by protoplasts or cells. This may be accomplished by, for instance, PEG or electroporation mediated-uptake, particle bombardment-mediated delivery, or microinjection. In each case the transformed cells may be regenerated to whole plants using standard techniques known in the art.

Techniques for the transformation of monocotyledon species include, but are not limited to, direct gene transfer into protoplasts using PEG or electroporation techniques, particle bombardment into callus tissue or organized structures, as well as Agrobacterium-mediated transformation.

The cells that have been transformed may be grown into plants in accordance with conventional techniques. See, for example, McCormick et al. (1986, Plant Cell Reports, 5:81-84). These plants may then be grown and evaluated for expression of desired phenotypic characteristics. These plants may be either pollinated with the same transformed strain or different strains, and the resulting hybrid having desired phenotypic characteristics identified. Two or more generations may be grown to ensure that the desired phenotypic characteristics are stably maintained and inherited and then seeds harvested to ensure stability of the desired phenotypic characteristics have been achieved.

Provided herein are methods for using a plant and/or plant material described herein. In one embodiment, a method includes using a plant and/or plant material. In one embodiment, a plant and/or plant material may be used to produce a plant material-derived product. Examples of plant material-derived products include lumber and pulp. Plant material-derived products may be used in, for instance, furniture making and construction. Plant material-derived products, such as pulp, may be used as a food additive, a liquid absorbent, as animal bedding, and in gardening. Plants and/or plant material described herein may also be used as a feedstock for livestock. Plants with reduced recalcitrance are expected to be more easily digested by an animal and more efficiently converted into animal mass. Accordingly, in one embodiment, a method include using a plant and/or plant material described herein as a source for a feedstock, and includes a feedstock that has plant material from a transgenic plant as one of its components.

In one embodiment, a method includes producing a metabolic product. A process for producing a metabolic product from a transgenic plant described herein may include processing a plant (also referred to as pretreatment of a plant), enzymatic hydrolysis, fermentation, and/or recovery of the metabolic product. Each of these steps may be practiced separately, thus included herein are methods for processing a transgenic plant to result in a pulp, methods for hydrolyzing a pulp that contain cells from a transgenic plant, and methods for producing a metabolic product from a pulp.

There are numerous methods or combinations of methods known in the art and routinely used to process plants. The result of processing a plant is a pulp. Plant material, which can be any part of a plant, may be processed by any means, including, for instance, mechanical, chemical, biological, or a combination thereof. Mechanical pretreatment breaks down the size of plant material. Biomass from agricultural residues is often mechanically broken up during harvesting. Other types of mechanical processing include milling or aqueous/steam processing. Chipping or grinding may be used to typically produce particles between 0.2 and 30 mm in size. Methods used for plant materials may include intense physical pretreatments such as steam explosion and other such treatments (Peterson et al., U.S. Patent Application 20090093028). Common chemical pretreatment methods used for plant materials include, but are not limited to, dilute acid, alkaline, organic solvent, ammonia, sulfur dioxide, carbon dioxide or other chemicals to make the biomass more available to enzymes. Biological pretreatments are sometimes used in combination with chemical treatments to solubilize lignin in order to make cell wall polysaccharides more accessible to hydrolysis and fermentation. In one embodiment, a method for using transgenic plants described herein includes processing plant material to result in a pulp. In one embodiment, transgenic plants described herein, such as those with reduced recalcitrance, are expected to require less processing than a control plant. In some embodiment, the conditions described below for different types of processing are expected to result in greater amounts of carbohydrate oligomers and carbohydrate monomers when used with a plant described herein compared to a control plant.

Steam explosion is a common method for pretreatment of plant biomass and increases the amount of cellulose available for enzymatic hydrolysis (Foody, U.S. Pat. No. 4,461,648). Generally, the material is treated with high-pressure saturated steam and the pressure is rapidly reduced, causing the materials to undergo an explosive decompression. Steam explosion is typically initiated at a temperature of 160-260° C. for several seconds to several minutes at pressures of up to 4.5 to 5 MPa. The biomass is then exposed to atmospheric pressure. The process typically causes degradation of cell wall complex carbohydrates and lignin transformation. Addition of H₂SO₄, SO₂, or CO₂ to the steam explosion reaction can improve subsequent cellulose hydrolysis (Morjanoff and Gray, 1987, Biotechnol. Bioeng. 29:733-741).

In ammonia fiber explosion (AFEX) pretreatment, biomass is treated with approximately 1-2 kg ammonia per kg dry biomass for approximately 30 minutes at pressures of 1.5 to 2 MPa. (Dale, U.S. Pat. No. 4,600,590; Dale, U.S. Pat. No. 5,037,663; Mes-Hartree, et al. 1988, Appl. Microbiol. Biotechnol., 29:462-468). Like steam explosion, the pressure is then rapidly reduced to atmospheric levels, boiling the ammonia and exploding the lignocellulosic material. AFEX pretreatment appears to be especially effective for biomass with a relatively low lignin content, but not for biomass with high lignin content such as newspaper or aspen chips (Sun and Cheng, 2002, Bioresource Technol., 83:1-11).

Concentrated or dilute acids may also be used for pretreatment of plant biomass. H₂SO₄ and HCl have been used at high concentrations, for instance, greater than 70%. In addition to pretreatment, concentrated acid may also be used for hydrolysis of cellulose (Hester et al., U.S. Pat. No. 5,972,118). Dilute acids can be used at either high (>160° C.) or low (<160° C.) temperatures, although high temperature is preferred for cellulose hydrolysis (Sun and Cheng, 2002, Bioresource Technol., 83:1-11). H₂SO₄ and HCl at concentrations of 0.3 to 2% (wt/wt) and treatment times ranging from minutes to 2 hours or longer can be used for dilute acid pretreatment.

Hot water can also be used as a pretreatment of plant biomass (Studer et al, 2011, Proc. Natl. Acad. Sci., U.S.A., 108:6300-6305). In one embodiment, hydrothermal treatment is at a temperature between 130° C. and 200° C., such as 140° C., 160, or 180° C., and for a time between 5 minutes and 120 minutes. In one embodiment, examples of times include at least 5 minutes, at least 10 minutes, at least 20 minutes, at least 30 minutes, at least 45 minutes, or at least 60 minutes. In one embodiment, examples of times include no greater than 120 minutes, no greater than 105 minutes, no greater than 90 minutes, or no greater than 75 minutes. The temperature and time used depends upon the source and condition of the biomass used, and an effective combination of time and temperature can be easily determined by the skilled person. In one embodiment, the biomass is exposed to a hydrothermal pretreatment having a severity level of log R0 between 2 and 5, where severity is defined as R0=t*exp ((T−100)/14.73) with t the time in minutes and T the temperature in degree Celsius (Lloyd and Wyman, 2005, Bioresource Technology, 96(18):1967-1977; Overend and Chornet, 1987, Phil. Trans. R. Soc. Lond. (A321), 523-536; and Wyman and Kumar, US Published Patent Application 20110201084). Examples of severity levels include at least 2, at least 2.5, at least 3, at least 3.5, at least 4, at least 4.5, and at least 5.

Other pretreatments include alkaline hydrolysis (Qian et al., 2006, Appl. Biochem. Biotechnol., 134:273; Galbe and Zacchi, 2002, Appl. Microbiol. Biotechnol., 59:618), oxidative delignification, organosolv process (Pan et al., 2005, Biotechnol. Bioeng., 90:473; Pan et al., 2006, Biotechnol. Bioeng., 94:851; Pan et al., 2006, J. Agric. Food Chem., 54:5806; Pan et al., 2007, Appl. Biochem. Biotechnol., 137-140:367), or biological pretreatment.

Methods for hydrolyzing a pulp may include enzymatic hydrolysis. Enzymatic hydrolysis of processed biomass may include the use of cellulases. Some of the pretreatment processes described above include hydrolysis of complex carbohydrates, such as hemicellulose and cellulose, to monomer sugars. Others, such as organosolv, prepare the substrates so that they will be susceptible to hydrolysis. This hydrolysis step can in fact be part of the feimentation process if some methods, such as simultaneous saccharification and fermentation (SSF), are used. Otherwise, the pretreatment may be followed by enzymatic hydrolysis with cellulases.

A cellulase may be any enzyme involved in the degradation of the complex carbohydrates in plant cell walls to fermentable sugars, such as glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, e.g., hydrolyze cellulose, either in the acid, neutral, or alkaline pH-range.

A cellulase may be of fungal or bacterial origin, which may be obtainable or isolated from microorganisms which are known to be capable of producing cellulolytic enzymes. Useful cellulases may be produced by fermentation of the above-noted microbial strains on a nutrient medium containing suitable carbon and nitrogen sources and inorganic salts, using procedures known in the art.

Examples of cellulases suitable for use in the present invention include, but are not liminted to, CELLUCLAST (available from Novozymes A/S) and NOVOZYME (available from Novozymes A/S). Other commercially available preparations including cellulase which may be used include CELLUZYME, CEREFLO and ULTRAFLO (Novozymes A/S), LAMINEX and SPEZYME CP (Genencor Int.), and ROHAMENT 7069 W (Rohm GmbH).

The steps following pretreatment, e.g., hydrolysis and fermentation, can be performed separately or simultaneously. Conventional methods used to process the plant material in accordance with the methods disclosed herein are well understood to those skilled in the art. Detailed discussion of methods and protocols for the production of ethanol from biomass are reviewed in Wyman (1999, Annu. Rev. Energy Environ., 24:189-226), Gong et al. (1999, Adv. Biochem. Eng. Biotech., 65: 207-241), Sun and Cheng (2002, Bioresource Technol., 83:1-11), and Olsson and Hahn-Hagerdal (1996, Enzyme and Microb. Technol., 18:312-331). The methods of the present invention may be implemented using any conventional biomass processing apparatus (also referred to herein as a bioreactor) configured to operate in accordance with the invention. Such an apparatus may include a batch-stirred reactor, a continuous flow stirred reactor with ultrafiltration, a continuous plug-flow column reactor (Gusakov, A. V., and Sinitsyn, A. P., 1985, Enz. Microb. Technol., 7: 346-352), an attrition reactor (Ryu, S. K., and Lee, J. M., 1983, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Appl. Biochem. Biotechnol., 56: 141-153). Smaller scale fermentations may be conducted using, for instance, a flask.

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC). The fermentation can be carried out by batch fermentation or by fed-batch fermentation.

SHF uses separate process steps to first enzymatically hydrolyze plant material to glucose and then ferment glucose to ethanol. In SSF, the enzymatic hydrolysis of plant material and the fermentation of glucose to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the coferementation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbiol. Mol. Biol. Reviews, 66: 506-577).

The final step may be recovery of the metabolic product. Examples of metabolic products include, but are not limited to, alcohols, such as ethanol, butanol, a diol, and organic acids such as lactic acid, acetic acid, formic acid, citric acid, oxalic acid, and uric acid. The method depends upon the metabolic product that is to be recovered, and methods for recovering metabolic products resulting from microbial fermentation of plant material are known to the skilled person and used routinely. For instance, when the metabolic product is ethanol, the ethanol may be distilled using conventional methods. For example, after fermentation the metabolic product, e.g., ethanol, may be separated from the fermented slurry. The slurry may be distilled to extract the ethanol, or the ethanol may be extracted from the fermented slurry by micro or membrane filtration techniques. Alternatively the fermentation product may be recovered by stripping.

The present invention is illustrated by the following examples. It is to be understood that the particular examples, materials, amounts, and procedures are to be interpreted broadly in accordance with the scope and spirit of the invention as set forth herein.

### Example 1

### 4-O-Methylation of Glucuronic Acid in Glucuronoxylan is Catalyzed by a DUF Family 579 Protein

4-O-methyl glucuronoxylan is one of the principle components present in the secondary cell walls of eudicotyledonous plants. However, the biochemical mechanisms leading to the formation of this hemicellulosic polysaccharide and the effects of modulating its structure on the physical properties of the cell wall are poorly understood. Described herein is the identification and functional characterization of an Arabidopsis glucuronoxylan methyltransferase (GXMT) that catalyzes 4-O-methylation of the glucuronic acid substituents of this polysaccharide. AtGXMT1, which was previously classified as a Domain of Unknown Function (DUF) 579 protein, specifically transfers the methyl group from S-adenosyl-L-methionine to O-4 of α-D-glucopyranosyluronic acid residues that are linked to O-2 of the xylan backbone. Biochemical characterization of the recombinant enzyme indicates that GXMT1 is localized in the Golgi apparatus and requires Co²⁺ for optimal activity in vitro. Plants lacking GXMT1 synthesize glucuronoxylan in which the degree of 4-O-methylation is reduced by 75%. This is correlated to a change in lignin monomer composition and an increase in glucuronoxylan release during hydrothermal treatment of secondary cell walls. It is proposed that the DUF579 proteins constitute a family of cation-dependent, polysaccharide-specific O-methyl-transferases. This knowledge provides new opportunities to selectively manipulate polysaccharide O-methylation and extends the portfolio of structural targets that can be modified either alone or in combination to modulate biopolymer interactions in the plant cell wall.

### Materials and Methods

Plant Materials and Mutant Identification. All A. thaliana plants were in the Columbia (Col-0) background. Seeds of T-DNA insertion lines (SALK_{—}018081, gxmt1-1; SALK_{—}087114, gxmt1-2) were obtained from the Arabidopsis Biological Resource Center (www.arabidopsis.org). Plants were grown for 8 weeks under short-day conditions (12 h photoperiod) at 22° C., 50% relative humidity and a light intensity of ˜180 μmol photons m⁻² s⁻¹. (For details see Example 2).

Preparation and Analysis of the Cell Wall Polysaccharides. Details of cell wall preparation and analyses are described in SI Materials and Methods.

Generation of GST-GXMT1 Fusion Protein. The GXMT1 protein was expressed in E. coli BL21-CodonPlus (DE3)-RIPL cells with an N-terminal glutathione S-transferase tag (GST-GXMT1). Details of generation, expression and purification of the GST-GXMT1 fusion protein are described in Example 2.

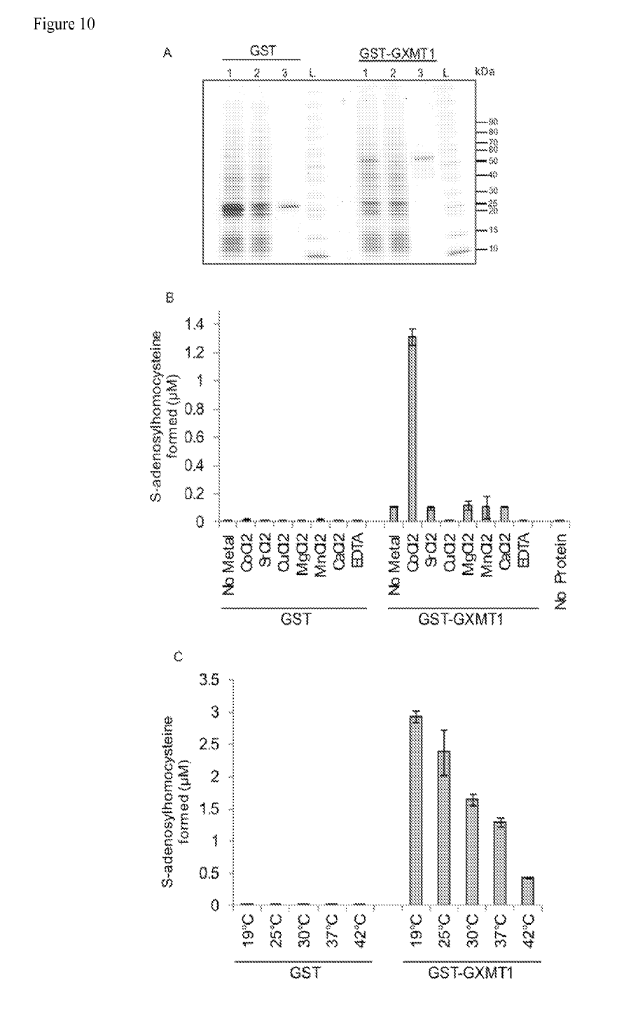

Determination of Methyltransferase Activity using ¹H-NMR Spectroscopy and LC-ESI-MS. The temperature optimum for GXMT1 activity was between 19-25° C. (FIG. 10C). The transfer of methyl groups to O-4 of GlcA was established by ¹H NMR spectroscopy. Assays were performed at 23° C. in 50 mM potassium bicarbonate, pH 7.5, (250 μL) containing acceptor substrate equivalent to 2.27 mM available GlcA residues, recombinant GXMT1 (10 μM), CoCl₂ (2 mM) and 1.5 mM S-adenosyl-L-methionine sulfate p-toluenesulfonate, unless otherwise indicated. The formation of SAH from SAM was determined using LC-ESI-MS (Salyan et al., 2006, Anal Biochem 349:112-117). Assays were performed in 50 mM HEPES, pH 7.5 (100 μL) with recombinant GXMT1 (3.4 μM), gxmt1-1 xylan polymer (220 μg), CoCl₂ (1 mM) and various amounts of SAMe-PTS. Details of both assays are in Example 2.

Subcellular Localization of GXMT1. Vector construction for the N-terminal fusion of GXMT1 to YFP, transient expression in N. benthamiana and confocal microscopy are described in Example 2. Marker proteins for ER (ER-ck), Golgi apparatus (G-ck), and PM (pm-ck) fused to CFP have been described (Nelson et al., 2007, Plant J 51:1126-1136).

Glucose and Xylose Release from Arabidopsis AIR by Hydrothermal Pretreatment and Enzymatic Hydrolysis. The amounts of glucan and xylan in Arabidopsis stem AIR were determined as described (DeMartini et al., 2011, Biotechnol Bioeng 108:306-312). Hydrothermal pretreatment and enzymatic hydrolysis of Arabidopsis stem AIR were performed as described in Example 2.

Determination of the Lignin Monomer Composition of Arabidopsis AIR by HSQC NMR Spectroscopy. AIR from ball-milled Arabidopsis stems was used for the preparation of the lignin enriched material for NMR analyses. See Example 2 for details.

Indirect Immunofluorescence Microscopy of Arabidopsis Stems using Xylan Binding Modules as Molecular Probes. Previously published protocols were used to construct, express, and purify CBM35 (Bolam et al., 2004, J Biol Chem 279:22953-22963) and CBM2b-1-2 (Bolam et al., 2001, Biochemistry 40:2468-2477). Tissue preparation, CBM labeling and microscopy of six week old Arabidopsis stem sections were as described (Pattathil et al., 2010, Plant Physiol 153:514-525). Details are in Example 2.

### Results and Discussion

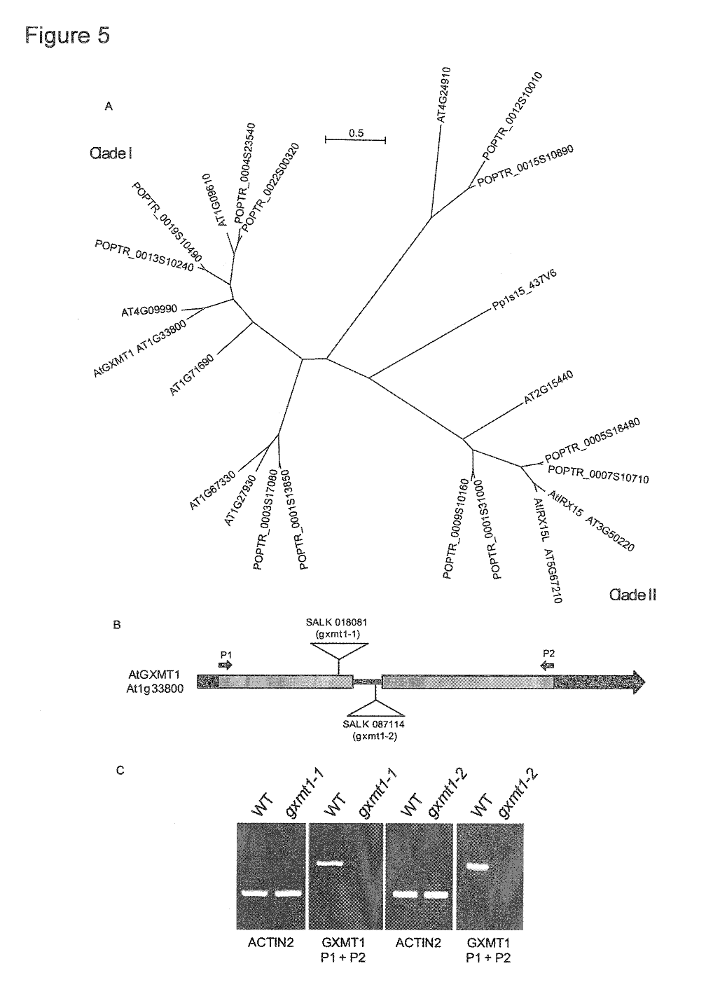

Methyl-etherification of Glucuronoxylan is Reduced in GXMT1 Mutants. Arabidopsis proteins that contain a Pfam PF04669 domain (Finn et al., 2010, Nucleic Acids Res 38:D211-D222), also known as Domain of Unknown Function 579 (DUF579), have been implicated in secondary cell wall development (Brown et al., 2005, Plant Cell 17:2281-2295, Oikawa et al., 2010, PLoS ONE 5:e15481, Brown et al., 2011, Plant J 66:401-413, Jensen et al., 2011, Plant J 66:387-400, Ruprecht et al., 2011, Front Plant Sci 2:23). The DUF579 family includes four phylogenetic clades (FIG. 5A). Two genes (At1g33800 and At1g09610) encoding previously uncharacterized members of Clade I are co-expressed with several other genes predicted to be involved in xylan synthesis including IRX7, IRX8, IRX9, IRX10, IRX15 and IRX15L (Brown et al., 2005, Plant Cell 17:2281-2295, Brown et al., 2011, Plant J 66:401-413, Jensen et al., 2011, Plant J 66:387-400). To investigate the role of GXMT1 in GX biosynthesis we isolated and characterized two homozygous T-DNA insertional alleles (SALK_{—}018081, gxmt1-1; SALK_{—}087114, gxmt1-2; FIG. 5B) in which At1g33800 is disrupted (FIG. 5C).

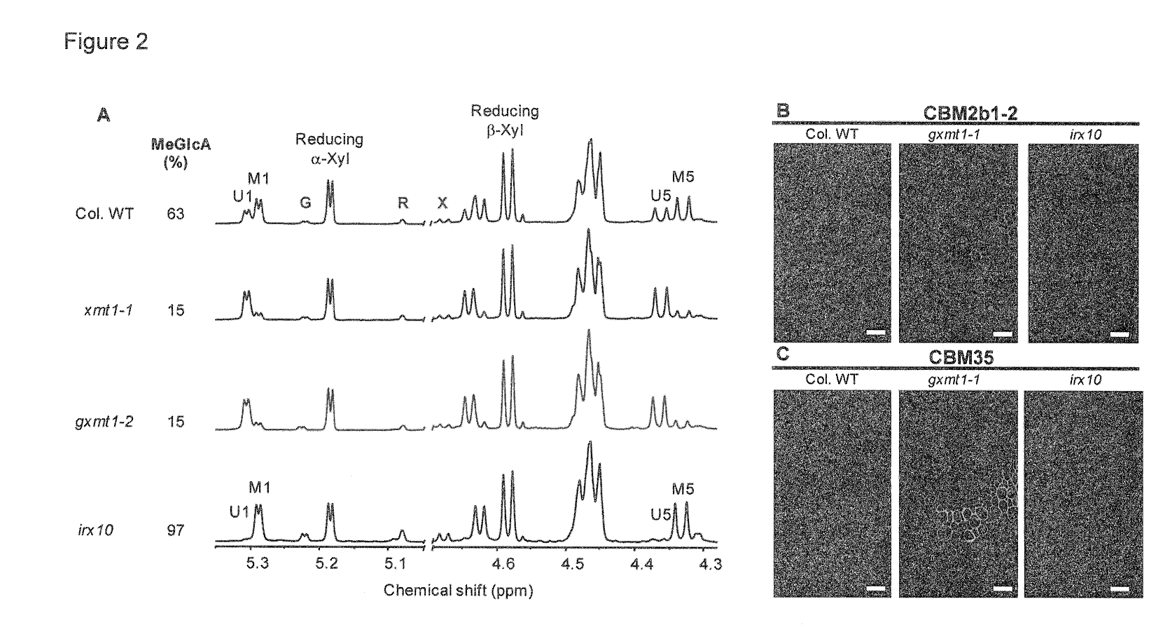

To identify and characterize changes in cell wall polysaccharide structure in GXMT1 mutants, fractions enriched in pectic and hemicellulosic polysaccharides were isolated from mature inflorescence stems, which are rich in secondary cell walls. ¹H NMR spectroscopy (Peña et al., 2007, Plant Cell 19:549-563) was used to compare the structures of the GX released by 1 N KOH-treatment of the alcohol insoluble residues (AIR) from inflorescence stems of wild-type, gxmt1-1, gxmt1-2 and irregular xylem 10 (irx10) plants. The irx10 mutant has a well-established xylan chemotype (Wu et al., 2009, Plant J 57:718-731) and served as a control. The ¹H-NMR spectra of the endo-xylanase-generated GX oligosaccharides (FIG. 2A) showed that the degree of GlcA O-methylation was 75% lower in both gxmt1-1 and gxmt1-2 plants than in wild-type plants and confirmed that GX produced by irx10 has a reduced chain length and contains almost exclusively methylated GlcA (Wu et al., 2009, Plant J 57:718-731). The amounts and distribution of branching and the degree of polymerization were indistinguishable for the GX from the GXMT1 mutants and wild-type plants. Together, these data suggest that GXMT1 is involved in 4-O-methyl etherification of the GlcA residues of GX.

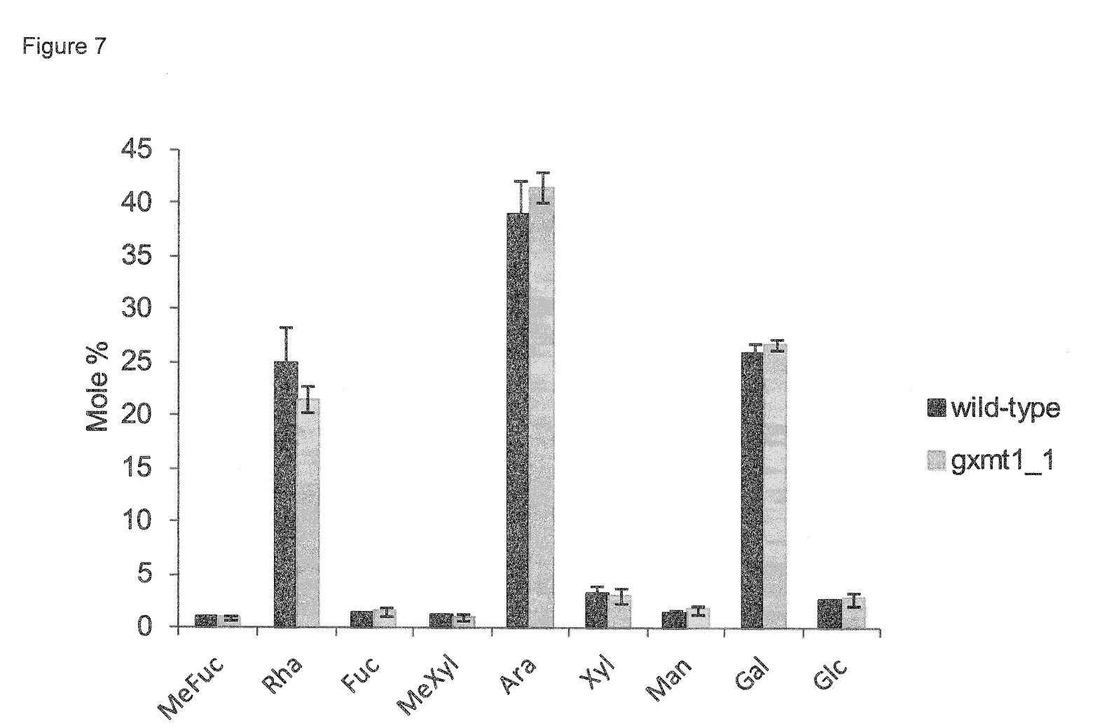

The pectic polysaccharide rhamnogalacturonan II contains 2-O-methyl-fucose and 2-O-methyl xylose (O'Neill et al., 2004, Annu Rev Plant Biol 55:109-139). Comparable amounts of these methyl-etherified sugars were present in the pectic polysaccharides from gxmt1-1 and wild-type plants (FIG. 7). Although 4-O-methyl-GlcA is known to be a component of arabinogalactan proteins in diverse plant species (Gaspar et al., 2001, Plant Mol Biol 47:161-176), we did not explore the effects of mutating GXMT1 on the structures of these polymers. GX is the only polysaccharide that we examined whose O-methylation is affected in gxmt1-1 plants.

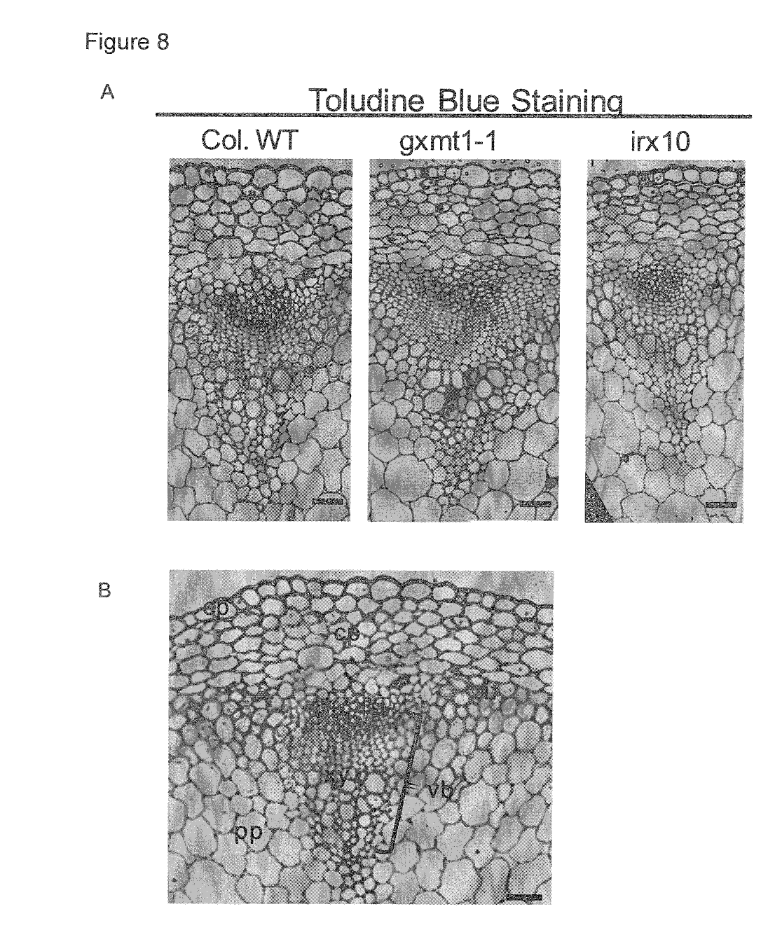

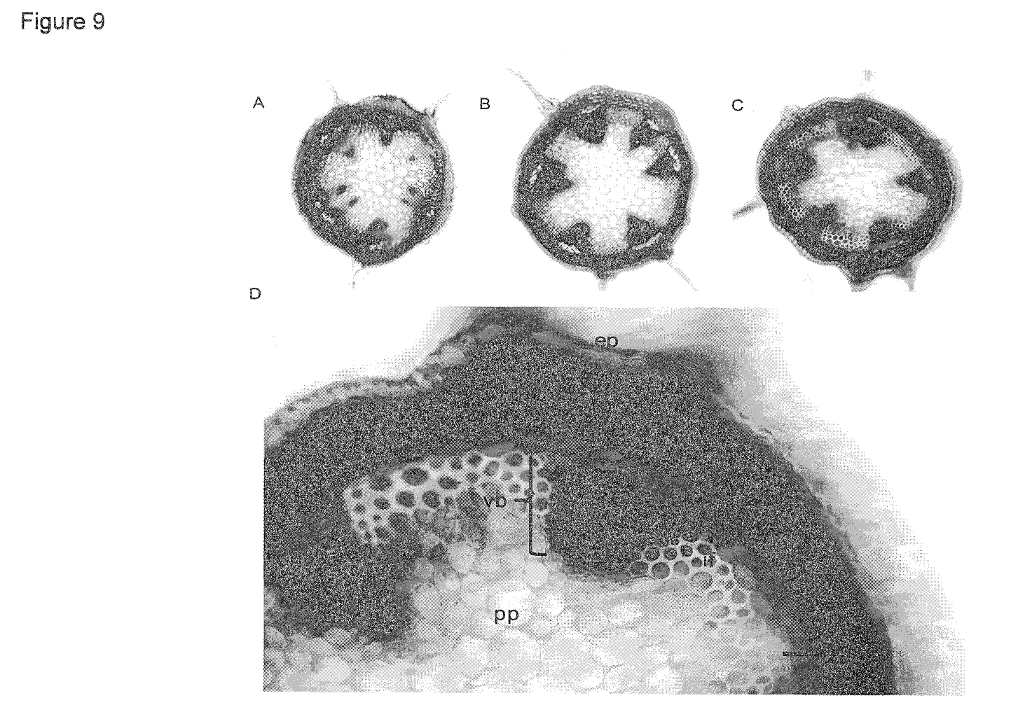

Although several Arabidopsis mutant lines, such as irx10, have altered xylan structure leading to collapsed xylem and interfascicular fibers with reduced wall thickness (Peña et al., 2007, Plant Cell 19:549-563, Brown et al., 2005, Plant Cell 17:2281-2295, Brown et al., 2007, Plant J 52:1154-1168, Wu et al., 2009, Plant J 57:718-731), gxmt1-1 stem sections are morphologically indistinguishable from wild-type stems (FIG. 8). Nevertheless, gxmt1-1 stems contain GX that is distinct from wild-type GX, with reduced methylation as shown by cytochemical analysis using non-catalytic carbohydrate binding modules (CBM). One of these, CBM2b-1-2, which binds to the backbone of linear and substituted xylans (McCartney et al., 2006, Proc Nat Acad Sci USA 103:4765-4770), extensively labels the GX-rich secondary walls of interfascicular fibers and vascular bundles in both gxmt1-1 and wild-type stems (FIG. 2B). As expected, less CBM2b1-2 labeling was observed in irx10 stems (FIG. 2B), which display a collapsed xylem phenotype due to decreased amounts of GX (Wu et al., 2009, Plant J 57:718-731). Conversely, CBM35 binds to GlcA but not to 4-O-methyl-GlcA substituents of GX (Montanier et al., 2009, Proc Nat Acad Sci USA 106:3065-3070). CBM35 and CBM2b1-2 displayed comparable labeling intensity in the walls of interfascicular fibers in the wild-type stems (FIGS. 2B and C). However, secondary walls of vascular xylem cells in these sections were weakly labeled with CBM35 (FIG. 2C), demonstrating that the GX in wild-type vascular xylem is highly methylated. Consistent with the almost complete methylation of GX in irx10 walls (FIG. 2A), no binding of CBM35 was observed (FIG. 2C). Notably, all secondary walls of gxmt1-1 stems were strongly labeled by CBM35. This binding was especially pronounced in xylem cells in vascular bundles (FIG. 2C), confirming that the GX in these tissues has a much lower degree of methylation relative to wild-type. These data are supported by analysis of transgenic pGXMT1::GUS lines (FIG. 9), which showed that the GXMT1 promoter is active predominantly in vascular bundles of mature stems.

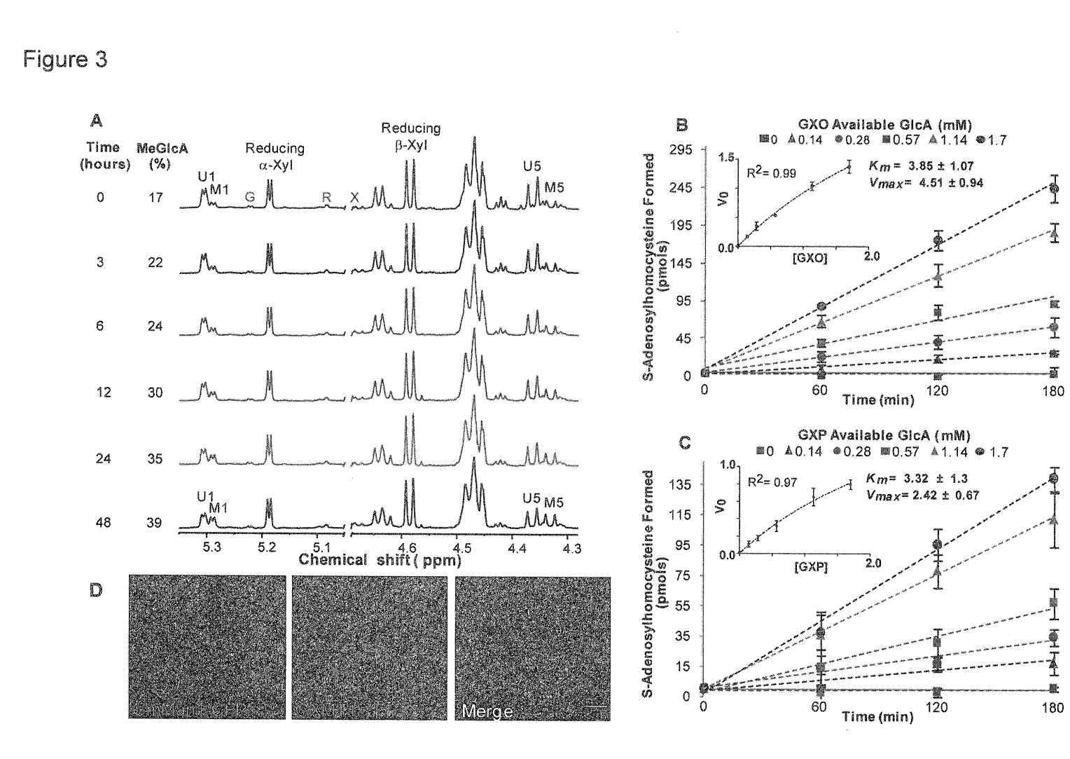

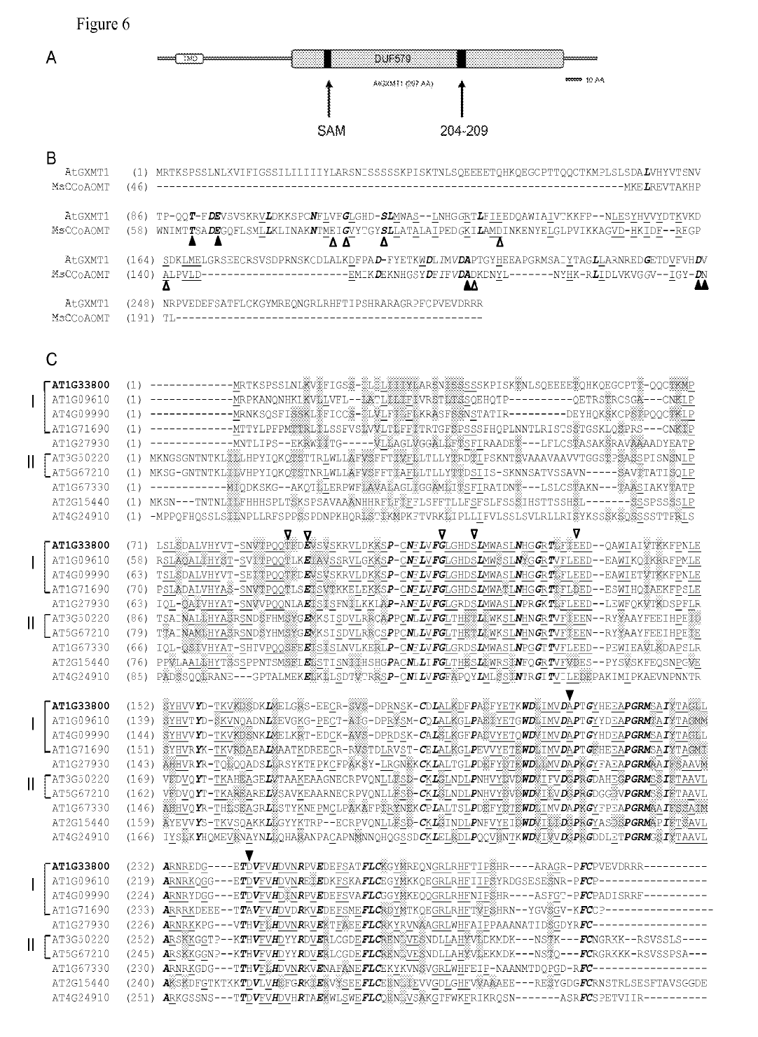

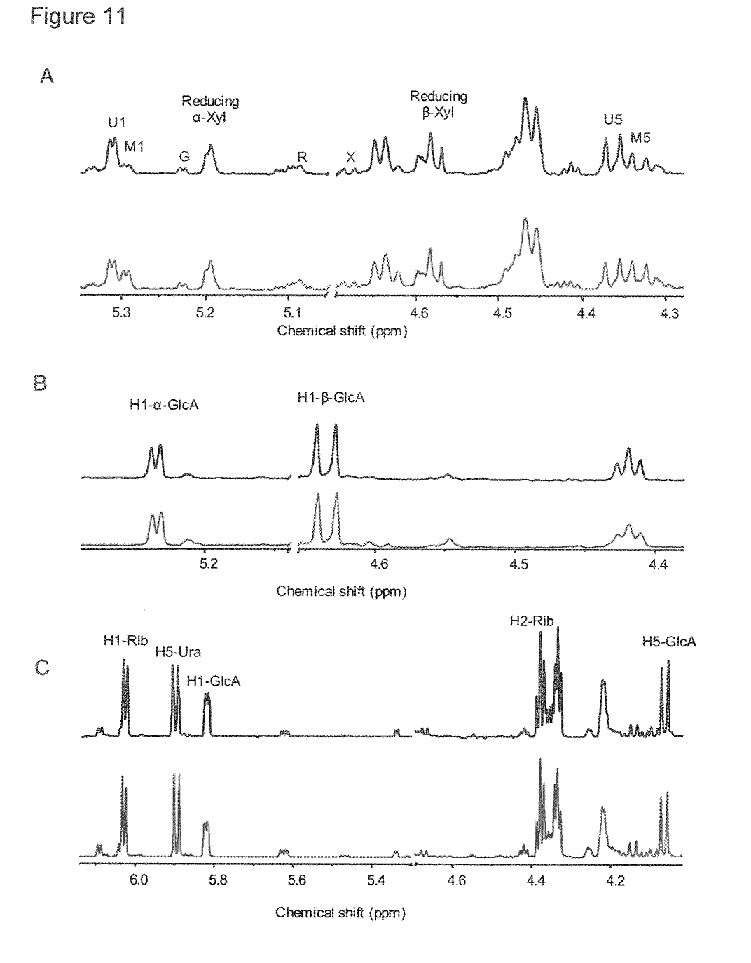

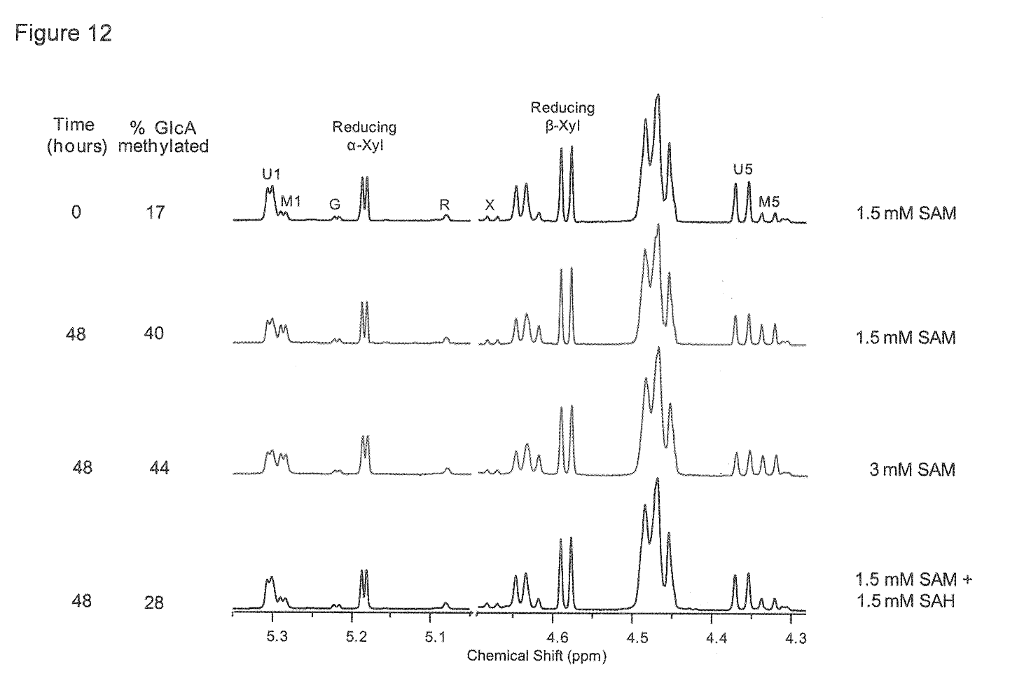

GXMT1 is a Glucuronoxylan-Specific Cation-Dependent 4-O-Methyltransferase. Our bioinformatic, spectroscopic and histochemical analyses led us to hypothesize that GXMT1 is a GX methyltransferase. Thus, a recombinant tagged form of GXMT1 (amino acids 44-297, see FIG. 6) was expressed in Escherichia coli, purified and tested for its ability to transfer the methyl group from SAM to various acceptor substrates (FIG. 10A). As it was not known if GlcA is methylated at the nucleotide sugar level or after its transfer to the xylan backbone, we evaluated a selection of potential GXMT1 acceptor substrates including GlcA, UDP-GlcA and sparsely methylated GX isolated from the gxmt1-1 mutant. After 48 h, the products formed were structurally characterized by 1D and 2D ¹H NMR spectroscopy to determine if O-methylation of the acceptor substrates had occurred. Our results establish that GXMT1 catalyzes the transfer of methyl groups exclusively to O-4 of GlcA in gxmt1-1 GX and its fragment oligosaccharides (FIG. 3A and FIG. 11). No methyl groups were transferred to GlcA or UDP-GlcA (FIG. 11), indicating that methylation occurs after addition of GlcA to the xylan backbone. The rate of methyl transfer to polymeric gxmt-1 xylan decreased after the first 3 h of the reaction (FIG. 3A) and after 48 h the degree of methylation had increased to 40%, which is somewhat less than the degree of methylation in wild-type GX. This is likely due to inhibition by S-adenosyl-L-homocysteine (SAH), the end-product of the reaction and a strong competitive inhibitor of many SAM-dependent methyltransferases (Moffatt and Weretilnyk, 2001, Physiol Plant 113:435-442). Indeed, we found that in vitro GXMT1 activity is inhibited by adding SAH at the start of the reaction (FIG. 12). In vivo, plants utilize SAH hydrolase (EC 3.3.1.1) and adenosine kinase (EC 2.7.1.20) to metabolize SAH, thus circumventing its inhibitory effects and promoting SAM regeneration and methyltransferase activities (Pereira et al., 2007, J Exp Bot 58:1083-1098).

To extend our knowledge regarding the biochemical properties of GXMT1, we adapted a liquid chromatography-electrospray ionization mass spectroscopy (LC-ESI-MS) method QSalyan et al., 2006, Anal Biochem 349:112-117, to detect and quantify GX 4-O-methyltransferase activity. This technique, which quantifies a product of the OMT reaction (SAH) with a detection limit of 60.25 nM and a linear response up to 3 μM, was used to show that recombinant GXMT1 exhibits similar Kₘ and Vₘₐₓ values for GX and its oligosaccharide fragments (FIGS. 3B and 3C). The Vₘₐₓ and Kₘ can only be approximated, as the acceptor substrate is not soluble at concentrations above the estimated Kₘ.

Previous assays of xylan methyltransferase activity using crude microsomal membranes suggest that xylan methylation is enhanced by certain divalent cations and inhibited by EDTA (Kauss and Hassid, 1967, J Biol Chem 242:1680-1685, Baydoun et al., 1989, Biochem J 257:853). We used the LC-ESI-MS based assay to evaluate the 4-O-methyltransferase activity of metal-depleted GXMT1 in the presence of Co²⁺, Sr²⁺, Cu²⁺, Mg²⁺, Mn²⁺, Ca²⁺ or EDTA. These analyses revealed that GXMT1-catalyzed transmethylation of GlcA substituents is a divalent metal-dependent process that is selectively potentiated by Co²⁺, enhancing GXMT1 activity an average of 1,180%. Enzyme activity was completely inhibited by Cu²⁺ and EDTA (FIG. 10B). These data suggest that 4-O-methylation of GlcA proceeds via a catalytic mechanism characteristic of plant Class I cation-dependent OMTs (Kopycki et al., 2008, J Mol Biol 378:154-164), consistent with an early report using a particulate enzyme from corn cobs (Kauss and Hassid, 1967, J Biol Chem 242:1680-1685). Plant cation-dependent OMTs typically require Mg²⁺, Ca²⁺, or Zn²⁺ for activity (Ferrer et al., 2005, Plant Physiol 137:1009-1017), although Co²⁺ can also enhance activity of selected OMTs (Luka\check{c}in et al., 2004, FEBS Lett 577:367-370). While several cobalt-dependent mammalian DNA N-methyltransferases have been described (Pfohl-Leszkowicz et al., 1987, Biochimie 69:1235-1242), GXMT1 is the only Co²⁺-dependent OMT described to date.

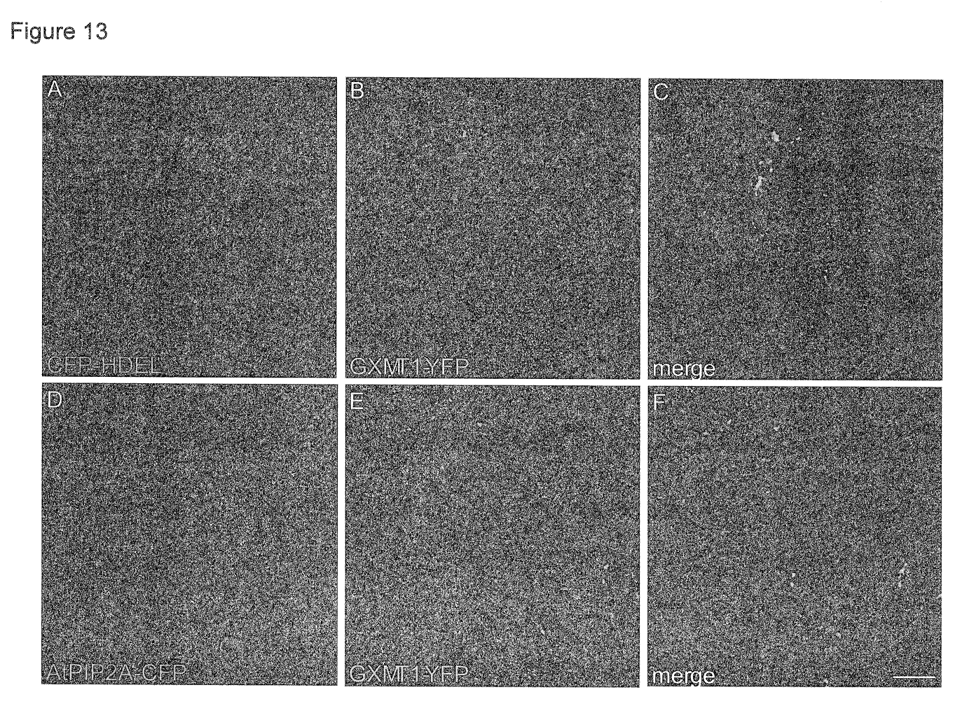

GXMT1 is Localized in the Golgi Apparatus. GXs are believed to be synthesized in the Golgi apparatus, but it is not known if they are O-methylated in this organelle (Scheller and Ulvskov, 2010, Annu Rev Plant Biol 61:263-289). Thus, we co-expressed GXMT1 fused to Yellow Fluorescent Protein (GXMT1-YFP) with several well-characterized organelle markers in Nicotiana benthamiana and performed live-cell confocal analysis (Nelson et al., 2007, Plant J 51:1126-1136). GXMT1-YFP fluorescence, which was observed within small, highly mobile puncta characteristic of tobacco leaf Golgi (Brandizzi et al., 2002, Plant Cell 14:1293-1309), co-localized with the Golgi marker GmMan1-CFP (G-ck) (FIG. 3D), but not with the endoplasmic reticulum (ER) marker CFP-HDEL (ER-ck) or the plasma membrane (PM) marker AtPIP2A-CFP (pm-ck) (FIG. 13). The SVMtm Transmembrane Domain Predictor (Yuan et al., 2004, J Comp Chem 25:632-636) predicts that GXMT1 has a single transmembrane domain spanning amino acids 13-31. Together, this suggests that xylan methylation occurs in the Golgi and is consistent with studies showing that other putative xylan biosynthetic enzymes are localized in this organelle (Peña et al., 2007, Plant Cell 19:549-563, Wu et al., 2009, Plant J 57:718-731, Brown et al., 2011, Plant J 66:401-413, Jensen et al., 2011, Plant J 66:387-400).

Potential Roles of Other DUF579 Proteins in Xylan Biosynthesis. IRX15 and IRX15L are two proteins in Clade II of the DUF579 family (FIG. 5A) that have been proposed to be involved in GX biosynthesis, although their biochemical functions are not known (Brown et al., 2011, Plant J 66:401-413, Jensen et al., 2011, Plant J 66:387-400). IRX15 and IRX15L share low sequence similarity (30% identity) with GXMT1. Nevertheless, several of the amino acid sequences predicted to function in divalent metal coordination and SAM/SAH binding are conserved in IRX15 and IRX15L, indicating that these proteins may function as OMTs (FIG. 6). However, a direct role for IRX15 and IRX15L in O-methylation of GX is difficult to reconcile with the observation that the degree of GlcA O-methylation is increased in irx15 and irx151 single mutants and that the irx15 irx151 double mutant (Brown et al., 2011, Plant J 66:401-413) produces a homodisperse, highly methylated GX with a reduced degree of polymerization (Brown et al., 2011, Plant J 66:401-413, Jensen et al., 2011, Plant J 66:387-400) similar to that found in irx9 and irx10 mutants. IRX9 and IRX10 are members of GT families GT43 and GT47, respectively, and have been implicated in xylan backbone elongation (Peña et al., 2007, Plant Cell 19:549-563, Wu et al., 2009, Plant J 57:718-731). Thus, the possibility cannot be discounted that IRX15 and IRX15L are structural rather than catalytic components of a putative xylan synthase complex. Non-catalytic GT homologs have been proposed to participate in the assembly of glycosyltransferase complexes involved in pectin synthesis (Atmodjo et al., 2011, Proc Nat Acad Sci USA 108:20225-20230). IRX15 and IRX15L may serve a similar role in xylan biosynthesis.

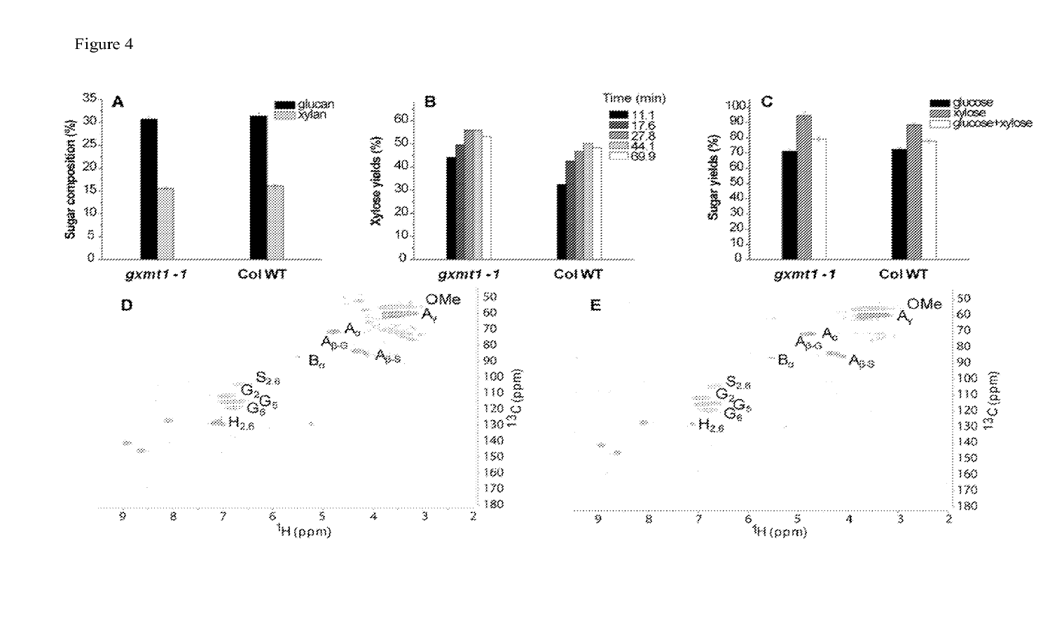

Mutating GXMT1 Enhances Xylan Release During Mild Hydrothermal Pretreatment. Engineering plant biomass to increase the accessibility of secondary cell wall components to enzyme-catalyzed hydrolysis may facilitate the conversion of biomass into fermentable sugars (Carroll and Somerville, 2009, Ann Rev Plant Biol 60:165-182, Himmel et al., 2007, Science 315:804-807). One promising approach is to alter the expression of genes that affect the molecular interactions of polymers responsible for the wall's structural integrity. For example, modulating the expression of OMTs involved in lignin biosynthesis has had success in decreasing the recalcitrance of plant biomass to enzyme-catalyzed saccharification (Chen and Dixon, 2007, Nature Biotechnol 25:759-761, Fu et al., 2011, Proc Nat Acad Sci USA 108:3803-3808). In contrast, the effects of manipulating O-methylation of GX are unknown. We therefore examined the effects of reduced O-methylation of GX on the release of xylose during hydrothermal pretreatment at several severities (Studer et al., 2010, Biotechnol Bioeng 105:231-238). Wild-type and gxmt1-1 plants contain comparable amounts of total glucan and xylan (FIG. 4A). However, hydrothermal pretreatment solubilized more xylan from gxmt1-1 AIR than from wild-type AIR (FIG. 4B). This difference was greatest when the least severe condition (11.1 min) was used. When this pretreatment was followed by cellulase and xylanase treatments, a greater proportion of the xylose and more total sugar was released from the gxmt1-1 AIR than from wild-type AIR (FIG. 4C). These data suggest that the molecular interactions holding GX in secondary walls are altered in gxmt1-1 plants and that mild hydrothemial pretreatment protocols that efficiently remove GX from such plants are feasible. Harsh pretreatments using mineral acids or high temperatures for extended times typically convert some of the GX to by-products that inhibit downstream processing by enzymes or microorganisms (Mosier et al., 2005, Biores Technol 96:673-686). Thus, biomass engineered to facilitate GX solubilization using mild hydrothermal conditions has potential as a feedstock that can be efficiently converted to fermentable sugars.

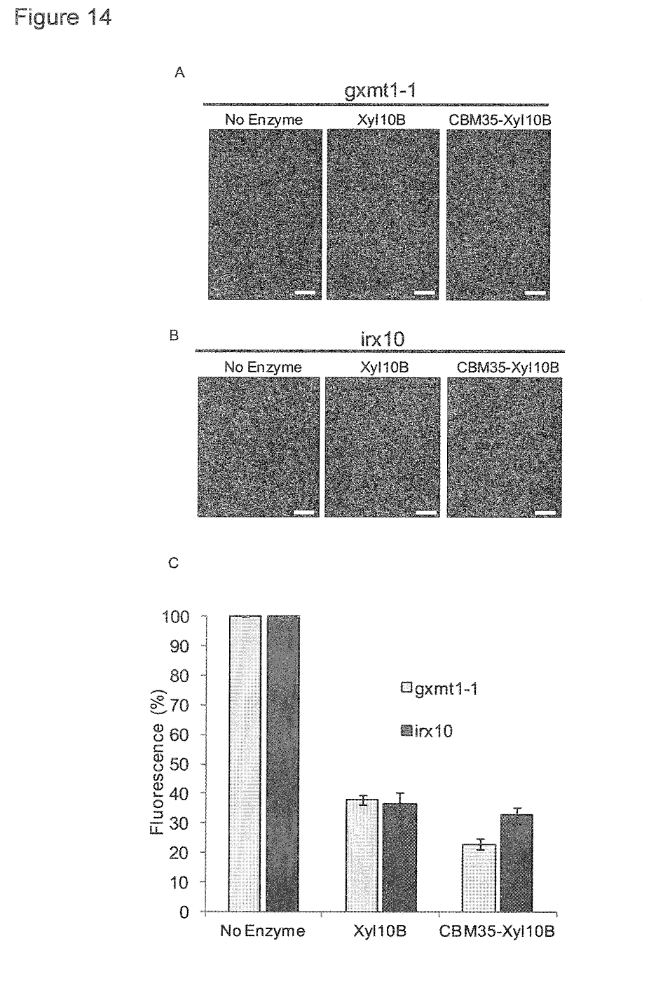

The selective removal of GX from biomass can be enhanced by using glycanases engineered to contain CBMs that target this polysaccharide (Hervé et al., 2010, Proc Nat Acad Sci USA 107:15293-15298). In this context, we demonstrated that a bacterial xylanase (Xyl10B) linked to CBM35 is more effective than the xylanase alone in fragmenting GX in the secondary cell walls of gxmt1-1 plants (FIG. 14). These results establish proof-of-principle for approaches that combine engineered secondary cell walls with designer endoglycanases to increase the efficiency of bioconversion technologies for lignocellulosic feedstocks.

Mutation of GXMT1 Results in Altered Lignin Structure. Patten et al. (Patten et al., 2010, Mol BioSyst 6:499-515) observed that the S-lignin is less abundant in Arabidopsis stem vascular bundles than in interfascicular fibers. Our data (FIGS. 2B and C) indicates that the degree of O-methylation of GX is higher in vascular bundles than in interfascicular fibers. This suggests that the degree of GX methylation is negatively correlated to the degree of lignin methylation. Indeed, HSQC NMR spectroscopy (Kim and Ralph, 2010, Org Biomol Chem 8:576-591) showed that the decrease in O-methylation of GX in gxmt1-1 plants is correlated to a ˜20% increase in the overall extent of lignin methylation, manifested as an increase in S lignin and a decrease in H lignin (FIGS. 4D and E). GX and lignin biosynthesis compete for a limited pool of SAM that is available during secondary cell wall synthesis. Therefore, the increase in lignin methylation observed in gxmt1-1 plants may reflect changes in metabolic flux associated with the decrease in GX methylation. Alternatively, O-methylation of GX may influence its association with the amphiphilic surface of lignin and/or the monolignols from which lignin is polymerized, thereby exerting a direct effect on lignin assembly in the cell wall.

### CONCLUSIONS

The results show that Arabidopsis GXMT1 encodes a GX-specific 4-O-methyltransferase responsible for methylating 75% of the GlcA residues in GX isolated from mature Arabidopsis inflorescence stems. Reduced methylation of GX in gxmt1-1 plants is correlated with altered lignin composition and increased release of GX by mild hydrothermal pretreatment. In addition to providing fundamental insights into cell wall synthesis, this discovery and characterization of AtGXMT1 extends the portfolio of structural targets that can be modified either alone or in combination to increase the economic value of lignocellulosic biomass. The ability to selectively manipulate polysaccharide O-methylation may provide new opportunities to modulate biopolymer interactions in the plant cell wall. The implications of our discovery are not limited to xylan biosynthesis, as other members of the DUF579 family may well catalyze the methyl-etherification of other plant polysaccharides.

### Example 2

### Details of Materials and Methods Used in Example 1

Plant materials and mutant identification. All Arabidopsis thaliana plants used were in the Columbia (Col-0) background. Seeds of T-DNA insertion lines (SALK_{—}018081, gxmt1-1; SALK_{—}087114, gxmt1-2) in AtGXMT1 (At1g33800) were obtained from the Arabidopsis Biological Resource Center (available using the world wide web at arabidopsis.org). Arabidopsis irx10 seeds (Wu et al., 2009, Plant J 57:718-731) were a gift of Alan Marchant (University of Southampton, England). A. thaliana plants were grown in Conviron growth chambers under short-day conditions (12 h photoperiod) at 22° C., 50% relative humidity, and a light intensity of ˜180 μmol photons m⁻² s⁻¹.

PCR analysis of genomic DNA isolated from individual gxmt1-1 and 1-2 plants was used to confirm the presence of the T-DNA insertion and the absence of the intact gene. The following primers were used: SALK_{—}018081_LP (5′-TGCAACTACCATGTTGGTTCC, SEQ ID NO:34), SALK_{—}018081_RP (5′-AGTTTCACCATCTTCACGGTTAC, SEQ ID NO:35) and LBb1.3 (5′-ATTTTGCCGATTTCGGAAC, SEQ ID NO:36). Transcript analysis of the mutants was performed using RNA extracted from stem tissue using the RNeasy Plant mini kit (Qiagen, Valencia, Calif.) with the DNaseI step. First strand cDNA was prepared from 1 μg of total RNA with the RevertAid First Strand cDNA Synthesis Kit (Fermentas, TherraroFisher, Waltham, Mass.) and Oligo(dT)₁₈ primer. The absence of GXMT1 transcript in gxmt1-1 and gxmt1-2 was verified by RT-PCR analysis using the following primer pair 1g33800_CDS_F(P1), 5′-ATGAGGACCAAATCTCCATCTTCTC/1g33800_CDS_R(P2), 5′-ACGGCGGCGATCAACTTCC (SEQ ID NO:37 and SEQ ID NO:38, respectively). See FIG. 5B for primer locations.

Phylogenetic analysis. The deduced amino acid sequence of Arabidopsis GXMT1 was used to search the publicly available databases at TAIR (http://www.arabidopsis.org/) and Phytozome v8.0 (http://www.phytozome.net). The identified protein sequences from Arabidopsis thaliana, Physcomitrella patens and Populus trichocarpa were downloaded and sequence alignments performed using the ClustalW2 algorithm (Larkin et al., 2007, Bioinformatics 23:2947-2948). An unrooted phylogenetic tree was built with SeaView v.3.3 (Gouy et al., 2010, Mol Biol Evol 27:221-224) using the maximum-likelihood PHYML 3 heuristic (Guindon and Gascuel, 2003, System. Biol. 52:696-704) within SeaView. The following settings were used: model, LG; invariable sites, optimized; across site rate variation, optimized; tree searching operations, best for Nearest Neighbor Interchanges (NNI) and Sub-tree Pruning and Regrafting (SPR) and BioNJ as the starting optimized tree topology. One hundred bootstrap replicates were completed to evaluate branch support.

Isolation and fractionation of stem alcohol insoluble residues. Arabidopsis stems from approximately 100 plants were harvested onto ice and kept at −80° C. The tissue was ground in liquid nitrogen to a powder with a mortar and pestle. The powder was suspended in aq. 80% (v/v) ethanol and then homogenized with a polytron (Kinematica Switzerland). The resulting slurry was filtered through 50 μm nylon mesh and the retentate washed with aq. 80% (v/v) ethanol. The insoluble residue was suspended in chloroform:methanol (1:1 v/v) and stirred for 1 h at room temperature. The suspension was filtered, the insoluble residue washed with acetone and air dried. The alcohol insoluble residues (AIR), which contains mainly cell walls, were then treated with enzymes and alkali to solubilize material enriched in pectin, xyloglucan and xylan (Zhong et al., 2005, Plant Cell 17:3390-3408). Briefly, AIR was treated with a mixture of Aspergillus niger endopolygalacturonase (EPG; Novozyme, Bagsvaerd, Denmark) and Aspergillus oryzae pectin methylesterase (PME; Novozyme,), then with a xyloglucan-specific endoglucanase (XEG; Novozymes) purified as described (Pauly et al., 1999, Glycobiology 9:93-100). The enzyme treated residue was then extracted with 1 N KOH containing 1% (w/v) NaBH₄ and then with 4 N KOH containing 1% (w/v) NaBH₄. The 1 and 4 N KOH soluble fractions were neutralized with glacial acetic acid, dialyzed against deionized water and lyophilized.

Analysis of cell wall polysaccharides. The materials solubilized by EPG and PME treatment of the AIR were desalted using a PD-10 gel filtration column (GE Healthcare, Fairfield, Conn.). The polysaccharides were converted to alditol acetate derivatives as described (Zhong et al., 2005, Plant Cell 17:3390-3408). Neutral sugars were identified and quantified by analysis of the derivatives by gas chromatography-electron-impact mass spectrometry (GC-MS) and GC-FID, respectively.

Glucuronoxylan oligosaccharides were generated by treating the 1 N KOH soluble material from stem AIR for 24 h at 37° C. with a Trichoderma viride endoxylanase (M1, 0.04 units/10 mg polysaccharide; Megazyme, Wicklow, Ireland). Ethanol was added to the reaction mixture (to 65% v/v) and the precipitate that formed removed by centrifugation. The glucuronoxylan-derived oligosaccharides remain in solution and were characterized by MALDI-TOF MS spectrometry and by ¹H NMR spectroscopy. The 4 N KOH extracts were treated with XEG to generate xyloglucan oligosaccharides, which were analyzed by MALDI-TOF mass spectrometry.

MALDI-TOF mass spectrometry. Positive-ion MALDI-TOF mass spectra were recorded using a Bruker Microflex LT mass spectrometer and Biospectrometry workstation (Bruker Daltonics, Billerica, Mass.). Aqueous samples (1 μL of a 1 mg/mL solution) were mixed with an equal volume of matrix solution (0.1 M 2,5-dihydroxbenzoic acid in aq. 50% methanol) and dried on the MALDI target plate. Typically, spectra from 150 laser shots were summed to generate each mass spectrum.

¹H-NMR Spectroscopy. Glucuronoxylan and xylo-oligosaccharides (1 to 2 mg) were dissolved in D₂O (0.25 mL, 99.9%; Cambridge Isotope Laboratories, Andover, Mass.). One- and two-dimensional NMR spectra were recorded at 298° K with a Varian Inova-NMR spectrometer (Agilent Technologies, Santa Clara Calif.) operating at 600 MHz for ¹H and equipped with a 5-mm NMR cold probe. Two-dimensional homonuclear gCOSY experiments were recorded using standard Varian pulse programs. The COSY spectra were collected as 800 1024 complex points. The data were processed with shifted squared sinebell window functions and zero filled to obtain a 2048 2048 matrix. Chemical shifts were measured relative to internal acetone (δ2.225). Data were processed using MestReNova software (Universidad de Santiago de Compostela, Spain).

Determination of the degree of polymerization of glucuronoxylan and the extent of GlcA methylation. The degree of polymerization of the glucuronoxylan was determined by analysis of the 1 and 2D ¹H NMR spectra of the endoxylanase generated oligosaccharides. Integrals of selected resonances in the 1-D spectra were used to determine the total amount of residues and the number of reducing ends in the glucuronoxylan (Para et al., 2007, Plant Cell 19:549-563). The extent of GlcA methylation was determined by integration of the signals corresponding to the anomeric protons of GlcA and methylated GlcA. The areas of the overlapping signals were determined using the deconvolution method in MestReNova.

Indirect immunofluorescence microscopy of Arabidopsis stems using xylan binding modules as molecular probes. Previously published protocols were used to construct, express, and purify CBM35 (Bolam et al., 2004, J Biol Chem 279:22953-22963) and CBM2b-1-2 (Bolam et al., 2001, Biochemistry 40:2468-2477). Both recombinant CBMs contain a His₆-tag. Inflorescence stems from six week old A. thaliana plants were prepared for immunofluorescence microscopy as described (Pattathil et al., 2010, Plant Physiol 153:514-525). For each experiment, 10-15 sections from three plants per line tested were used. Semi-thin transverse sections (250 nm) were cut from the basal portion of the stem with a Leica EM UC6 ultramicrotome (Leica Microsystems, Austria) and mounted on glass slides (colorfrost/plus, Fisher Scientific USA). Sections were blocked for 30 min with 3% (w/v) non-fat dry milk in 50 mM HEPES, pH 8.0, containing 2 mM CaCl₂. This buffer was used as calcium is required for CBM35 to bind to its ligand (Bolam et al., 2004, J. Biol. Chem., 279:22953-22963). No difference in CBM2b1-2 binding was observed in the presence and absence of calcium. The solution was removed and the sections treated overnight at 4° C. with CBM35 or CBM2b-1-2 (74 diluted to a concentration of 2 μM in HEPES-CaCl₂) and 3% (w/v) non-fat dry milk in HEPES-CaCl₂ (4 μL). The sections were washed three times with 10 mM Tris, pH 8.0, containing 150 mM NaCl₂ (5 min per wash). Mouse anti-His monoclonal antibody in Tris-NaCl (Sigma, 100-fold dilution) was applied and allowed to react for 1 h. The sections were washed three times with Tris-NaCl (5 min per wash) and then treated for 1 h in the absence of light with goat anti-mouse IgG (50-fold dilution in Tris-NaCl) coupled to Alexa Fluor 488 (Invitrogen). Sections were then washed twice with Tris-NaCl for 5 min, followed by distilled water. CITIFLUOR antifadant mounting medium AF1 (Electron Microscopy Sciences, Hatfield, Pa.) was applied and the sections covered with a cover slip. Light microscopy was carried out with a Nikon Eclipse 80i microscope as described (Pattathil et al., 2010, Plant Physiol 153:514-525).

GUS reporter gene analysis in Arabidopsis. The upstream region of the GXMT1 gene (At1g33800) was fused with the bacterial β-glucuronidase (GUS) gene by replacing the CaMV 35S promoter of pCAMBIA 1305.2 with promoter regions by ligation of restriction endonuclease digested PCR products into the GUS binary vector pCAMBIA 1305.2 (Cambia, Canberra, Australia). Arabidopsis genomic DNA, isolated from 5 day old leaves using the DNeasy Plant Mini Kit (Qiagen, Valencia, Calif.) was used as a template for PCR amplification of a 1538 bp region upstream of the start codon using the primer pair 1g33800_GUS_F-KpnI (5′-CGCGCGGTACCTGTCAGTGCCGTCAAG, SEQ ID NO:39) and 1g33800_GUS_R-NcoI (5′-CGCGCCCATGGTTTCTGACTAAAGAATCG, SEQ ID NO:40). Incorporated restriction sites are underlined.

Stable transformation of Arabidopsis Col-0 was performed using a floral dip method (Clough and Bent, 1998, Plant J. 16:735-743). T₁ plants were selected on plates containing 0.5× Murashige and Skoog basal medium with vitamins (PhytoTechnology Laboratories; Shawnee Mission, Kans.), 2-(N-morpholino)-ethanesulfonic acid (0.5% w/v), sucrose (0.8% w/v), agar (1.0% w/v) and hygromycin (50 mg/L). Seedlings were transferred to potting soil after 10 d, covered with saran wrap for 5 d to maintain a high humidity, uncovered and then allowed to self-pollinate. Histochemical analysis was performed on T2 plants.

GUS staining was performed as described (Sieburth and Meyerowitz, 1997, Plant Cell 9:355-365) with minor modifications. Tissue was incubated for 4 h at 37° C. in a staining solution consisting of 50 mM NaHPO₄, pH 7.2, containing Triton X-100 (0.2%), potassium ferrocyanide (2 mM), potassium ferricyanide (2 mM) and 2 mM X-Gluc (Gold Biotechnology, St. Louis, Mo.). Staining patterns were consistent across multiple independent lines. Hand-cut cross sections from representative plants were imaged under white light with a stereoscopic microscope (Olympus SZH-ILLD, Center Valley, Pa.) equipped with a digital camera (Nikon DS-Ril, Melville, N.Y.) and NIS-Elements Basic Research software (Nikon, Melville, N.Y.).

Subcellular localization of fluorescent protein-tagged proteins. GXMT1-YFP was generated by amplifying the full length coding sequence (without stop codons) by PCR from cDNA, prepared from Arabidopsis inflorescence stem tissue, using the following primer pair 5′-ATGAGGACCAAATCTCCATCTTCTC/5′-ACGGCGGCGATCAACTTCC (SEQ ID NO:41 and SEQ ID NO:42 respectively, and then cloned into the PCR8/GW/TOPO vector (Invitrogen, Carlsbad, Calif.) to create an Entry clone. The orientation of the CDS in the Entry clone was verified by PCR analysis followed by sequencing. To generate the vector for N-terminal fusions to YFP, the Entry clone was recombined into pEarlyGate101 using Gateway Technology (Invitrogen, Carlsbad, Calif.) via the LR-reaction (Earley et al., 2006, Plant J 45:616-629). pEarlyGate 101 clones were sequenced and transformed into Agrobacterium tumefaciens strain GV3101 by electroporation. Marker proteins for endoplasmic reticulum (CFP-HDEL, ER-ck), Golgi apparatus (GmMAN1-CFP, G-ck), and plasma membrane (AtPIP2A-CFP, pm-ck) have been described previously (Nelson et al., 2007, Plant J 51:1126-1136). A. tumefaciens was grown at 28° C. in YEB media supplemented with kanamycin (50 mg/L), rifampicin (50 mg/L) and gentamycin (25 mg/L). Cells were harvested by centrifugation (15 min at 2800×g) and then suspended to a final OD₆₀₀ of 0.5 in AS-medium (10 mM MES, pH 5.6, 10 mM MgCl₂, and 150 μM acetosyringone). Cell suspensions were kept at room temperature for 2 h prior to infiltration into leaves of 4-week-old Nicotiana benthamiana plants as described (Voinnet et al., 2003, Plant J 33:949-956). For co-infiltration, A. tumefaciens strains carrying different plasmids were mixed in a ratio of 1:1 to reach a final OD₆₀₀ of 1.0 prior to infiltration. Infiltrated leaves were imaged on a filter-based Olympus FV-1000 laser scanning confocal microscope (Olympus, Center Valley, Pa.) at 24, 48, 72 and 96 h post-infiltration. Maximal expression was observed at 72 h post infection. For assessment of protein co-localization, multiple 0.5 μm slices were taken through the z-plane using a 60× (N.A.1.2) water immersion objective. Due to the rapid mobility of the GXMT1-YFP+ structures, the highest scanning speeds were used and no Kalman corrections performed. Transient expression experiments and confocal microscopy were performed two independent times on multiple leaf samples. Images projections were generated using Image J (http://rsbweb.nih.gov/ij/) and processed in Adobe Photoshop (Adobe Systems, Mountain View, Calif.).

Generation of GST-GXMT1 fusion protein. GXMT1 was expressed with an N-terminal glutathione S-transferase tag (GST-GXMT1). The coding sequence of GXMT1 (amino acids 44-297) was amplified from Arabidopsis cDNA by PCR using the primers: GXMT1-EcoRI_F, 5′-GCGCGGAATTCAACAAATCTCTCCCAAGAAG, SEQ ID NO:43, and GXMT1-XhoIR, 5′-GCGCGCTCGAGACGGCGGCGATCAACTTC, SEQ ID NO:44 (the incorporated restriction sites are underlined). The PCR amplified cDNA subfragment was digested with EcoRI and XhoI and ligated in frame with GST in the pGEX-5X1 vector (GE Healthcare Fairfield, Conn.). The GST-GXMT1 fusion protein was overexpressed in E. coli BL21-CodonPlus (DE3)-RIPL cells (Agilent Technologies, Santa Clara, Calif.) by incubating the culture for 4 h at 27° C. in the presence of 80 μM isopropyl-β-thiogalactoside (IPTG) and 0.1× trace metals (Studier, 2005, Protein Expr Purif 41:207-234). Recombinant proteins were purified using a GSTrap-HP (GE Healthcare, Fairfield, Conn.) column according to the manufacturer's instructions. GST-GXMT1 was estimated to be at least 95% pure by SDS-PAGE with Coomassie Blue staining. Protein concentrations were estimated by measuring absorbance at 280 nm and using the calculated extinction coefficient (GST-GXMT1, 68030 M⁻¹ cm⁻¹; GST, 41060 M⁻¹ cm⁻¹). For activity assays using LC-ESI-MS, proteins were buffer exchanged into 50 mM HEPES, pH 7.5, using either a PD-10 gel filtration column (GE Healthcare, Fairfield, Conn.) or dialysis (3500 molecular weight cut-off).

Determination of methyltransferase activity using LC-ESI-MS. Glucuronoxylan 4-O-methyltransferase activity was determined using a liquid chromatography-electrospray ionization/mass spectroscopy (LC-ESI-MS) method using selective reaction monitoring (SRM) that measures the formation of S-adenosylhomocysteine from SAM (S17). LC-ESI-MS analyses were performed on a LTQ-XL linear ion trap mass spectrometer (ThermoFisher, Waltham, Mass.) equipped with a Surveyor MS Pump Plus HPLC, a Surveyor Autosampler Plus. Chromatographic separation was as described (Salyan et al., 2006, Anal Biochem 349:112-117). S-adenosylhomocysteine (SAH) eluted at 3.2 min. Thus, the material eluting between 2.5 and 5 min was transferred to the mass spectrometer using an in-line divert/inject valve. The selected reaction monitoring transition used was 385.1>250.1 with a collision energy of 20%. The detection limit was 60.25 nM S-adenosylhomocysteine with a linear response up to 3 μM. Xylan methyltransferase assays (100 μL) were performed in 50 mM HEPES, pH 7.5, containing recombinant enzyme (3.4 μM), gxmt1-1 xylan polymer (220 μg) and CoCl₂ (1 mM) unless otherwise indicated. Enzymatic reactions were equilibrated to the required temperature and then initiated by the addition of S-adenosyl-L-methionine sulfate p-toluenesulfonate (SAMe-PTS, Affymetrix, Santa Clara, Calif.) at the concentrations indicated. A 200 μM solution of SAMe-PTS in 50 mM HEPES, pH 7.5 was freshly prepared from a stock solution of SAMe-PTS (32 mM) in 5 μM sulfuric acid, 10% (v/v) ethanol, stored and kept at −20° C.

To investigate the divalent metal-dependence of GXMT1 activity, metal-depleted enzyme stock solutions and buffers were prepared by two treatments for 30 min at 4° C. with Chelex-100 ion exchange resin (Bio-Rad, Hercules, Calif.) and collected using Micro-Spin Columns (Pierce, Rockford, Ill.). The concentrations of metal salts or chelating agents, when added, are indicated in the figure legends. Reactions were performed for 3 h at 23° C., unless indicated otherwise, and terminated by the addition of formic acid to a final concentration 0.2% (v/v). Quenched reactions were centrifuged (13.3×g 10 min) to remove any precipitates that had formed and then transferred to glass Total Recovery vials (Waters, Milford, Mass.). A portion of the solution (5 μL) was then injected onto the column. Each injection series included a S-adenosylhomocysteine (Sigma) standard curve (6.25 nM to 3 μM) prepared similarly.

For determination of kinetic parameters, transferase reactions comprising 50 mM HEPES, pH 7.5, 3.4 μM enzyme, 1 mM CoCl₂ were performed at 23° C. for up to 180 min. Methyltransferase reactions were carried out using 150 μM SAMe-PTS as a donor substrate and six different concentrations of gxmt1-1 glucuronoxylan oligosaccharides (0-1.7 mM) or gxmt1-1 polymeric glucuronoxylan (0-0.17 mM), which is equivalent to 0-1.7 mM available GlcA residues. The average molecular weight of the oligomeric and polymeric glucuronoxylan were estimated by ¹H-NMR analysis to be 1268 g/mol and 17600 g/mol respectively with an average of 10 unmethylated GlcA residues present on each xylan polymer. To calculate initial rates, 200 aliquots were removed at regular time intervals, quenched by the addition of 50 μl of 1% (v/v) formic acid and the amount of S-adenosylhomocysteine formed determined by LC-ESI-MS. The steady state parameters Km and Vₘₐₓ were calculated by fitting the initial velocities to the Michaelis-Menten equation using nonlinear curve fitting in GraphPad Prism 5 (GraphPad Software, La Jolla, Calif.).

Monitoring methyltransferase activity by ¹H-NMR spectroscopy. Purified proteins were buffer exchanged into 50 mM potassium bicarbonate, pH 7.5, using a PD-10 gel filtration column (GE Healthcare) and then concentrated using a 10 kDa molecular weight cut-off Amicon Ultra centrifugal filter device (Millipore). The following substrates were tested for their ability to function as acceptors for GXMT1 at the indicated concentrations: gxmt1-1 xylan (1 mg), gxmt1-1 xylan oligosaccharides (2.27 mM), UDP-GlcA (2.27 mM, Sigma) and GlcA (2.27 mM, Sigma). Each reaction (250 μL) contained recombinant enzyme (10 μM), CoCl₂ (2 mM), SAMe-PTS (1.5 mM) and acceptor substrate at the concentrations indicated. Reactions were allowed to proceed at 23° C. for the indicated amount of time and then terminated by heating for 15 min at 100° C. Cobalt was removed prior to ¹H-NMR analysis by treating the heat-inactivated solutions for 30 min at 23° C. with Chelex-100 ion exchange resin (50 mg, BioRad), with end-over-end mixing. The solution was collected by centrifugation, transferred to a clean tube, heated for 5 min at 100° C. and treated twice more with fresh Chelex resin. The resin was removed from the solutions using Micro-Spin Columns (Pierce). The solutions were diluted to 1 mL with D₂O (99.98%, Cambridge Isotope Laboratories) and lyophilized. The dry reaction products were dissolved in 0.25 mL D₂O and analyzed by ¹H-NMR spectroscopy. Spectra were recorded before and after endoxylanase treatment, when polymeric xylan was used as a substrate.

The glucan and xylan content of Arabidopsis AIR. Glucan and xylan contents were determined using a downscaled compositional analysis method (DeMartini et al., 2011, Biotechnol Bioeng 108:306-312). The entire process was performed in 1.5 mL high recovery glass vials (Agilent, Santa Clara, Calif., USA) with 3 mg dry biomass, loaded into each vial by a Core Module Robotics Platform (Symyx Technologies, Sunnyvale, Calif.). A set of glucose and xylose standards was run in parallel to correct for sugar degradation. The released sugars were quantified using a Waters Alliance 2695 HPLC (Milford, Mass., USA) equipped with an Aminex HPX-87H column (BioRad, Hercules, Calif., USA) and a refractive index detector.

Pretreatment and enzymatic hydrolysis of Arabidopsis AIR. The amounts of glucose and xylose released by hydrothermal pretreatment and enzymatic hydrolysis of Arabidopsis stem AIR (4.5 mg) were determined as described (Studer et al., 2010, Biotechnol Bioeng 105:231-238, DeMartini and Wyman, 2011, Biores Technol 102:1352-1358). Hydrothermal pretreatment was conducted for 11.1 to 69.9 min at 180° C., which corresponds to severity factor ranging from 3.4 to 4.2 (Studer et al., 2010, Biotechnol Bioeng 105:231-238). A portion of the pretreated slurry was collected and centrifuged, and the soluble and insoluble materials subjected to acid hydrolysis for 1 h at 121° C. in sulfuric acid (4% by weight) to determine the sugar composition of the material released by pretreatment and present in the pretreated residue (DeMartini et al., 2011, Biotechnol Bioeng 108:306-312). The remaining pretreated slurry in 50 mM Na citrate, pH 4.8. was treated for 72 h at 50° C. with Accellerase® 1500 cellulase and Accellerase® XY xylanase (Genencor, Rochester, N.Y.) at a loading of 112.5 mg cellulase and 37.5 mg xylanase/g glucan+xylan in the biomass. The released sugars were quantified using an Agilent 1200 HPLC (Agilent, Santa Clara, Calif., USA) equipped with an Aminex HPX-87H column (BioRad, Hercules, Calif., USA) and a refractive index detector.

Production of xylanase-CBM fusion proteins. The CBM35-xylanase fusion protein was obtained by generating a gene construct encoding the CBM35 derived from Cellvibrio japonicus Abf62A fused to the Cellvibrio mixtus xylanase Xyl10B. The required pFV1-PT plasmid was constructed by modifying pET22b. Briefly, a 48-bp oligonucleotide adaptor encoding NdeI, KpnI, BamHI, HindIII, EcoRI, SacI, SalI, and XhoI was ligated into NdeI and XhoI-digested pET22b to generate pFV1. A DNA fragment encoding a 15-amino acid proline- and threonine-rich linker was cloned into HindIII- and EcoRI-digested pFV1 to generate pFV 1-PT. DNA sequences encoding CBM35 and the C-terminal catalytic domain of Xyl10B, were amplified by PCR incorporating appropriate terminal restriction sites. Amplified DNA encoding the catalytic domain was cloned into EcoRI- and XhoI-digested pFV 1-PT. The PCR product encoding CBM35 was cloned into BamHI-HindIII-digested pFV1-PT-xyll OB to generate genes encoding CBM35 fused to the xylanase. The expression and purification of the proteins were as described (Bolam et al., 2004, J Biol Chem 279:22953-22963, Bolam et al., 2001, Biochemistry 40:2468-2477).

Determination of xylanase and CBM35-xylanase activities in muro using immunofluorescence microscopy. Stem sections were treated for 2 h at room temperature with xylanase or CBM-xylanase (10 μM in Tris-NaCl). Control sections were treated with buffer only. The sections were then washed 3 times with Tris-NaCl (5 min each wash). For quantification of xylanase action, equivalent regions of the micrographs were selected for quantification. The extent of glucuronoxylan hydrolysis was assessed from the extent of binding of His-tagged CBM2b1-2 (10 μM) to plant sections using the labeling protocol described above. Enzymatic removal of xylan results in the disappearance of epitopes recognized by CBM2b1-2 and a corresponding reduction in the fluorescence signal. Control micrographs obtained without enzymatic treatment were designated as 100% of initial fluorescence, and fluorescence levels in micrographs of treated sections scaled accordingly. Fluorescence was measured using Image J software (http://rsbweb.nih.gov/ij/).

Determination of the lignin monomer composition of Arabidopsis AIR by HSQC NMR spectroscopy. Extractive free AIR from Arabidopsis stems was treated for 2 h in 30 min milling cycles followed by 30 min of rest in a vibrational ball-mill (Retsch, Newtown, Pa.). The milled material (100 mg) was suspended in 20 mM Na acetate, pH 5 (30 mL), and treated for 48 h at 35° C. and 200 rpm with Cellulysin (10 mg, EMD Chemicals, Gibbstown, N.J.). The solids were recovered by centrifugation (8000 g, 35° C., 30 min) and treated three more times (48 h each) with Cellulysin. The residue was recovered by centrifugation, suspended in 20 mM in potassium phosphate, pH 7, (30 mL), and then treated overnight at 37° C. with protease (5 mg, Sigma-Aldrich, St Louis, Mo.) to hydrolyze the remaining cellulases. The protease was deactivated by heating for 2 h at 90° C. The residue was washed extensively with 20 mM potassium phosphate, pH 7, then with deionized water and freeze-dried.

The lignin-enriched material was dissolved at 60° C. in perdeuterated pyridinium chloride-DMSO-d₆ (1:3 w/w). HSQC spectra were recorded at a sample temperature of 50° C. with a Bruker Avance-500 NMR spectrometer (Bruker, Billerica, Mass.) equipped with an xyz-gradient triple resonance probe for indirect detection. The spectral widths were 11.0 and 180.0 ppm for the ¹H and ¹³C dimensions, respectively. HSQC analysis was performed using a standard Bruker gradient-enhanced pulse sequence optimized for a ¹J_{C-H} of 145 Hz with a 90° pulse of 5 ms, an acquisition time of 0.11 s and a relaxation delay of 1.5 s. Data for 256 transients were recorded as 256 complex data points for each single-quantum evolution time. HSQC cross peak assignments are annotated using the nomenclature of Kim and Ralph (Goujon et al., 2010, Nucleic Acids Res 38:W695-W699).

### Example 3

As described in Example 1, we have demonstrated that an Arabidopsis gene (At1g33800) encodes a cation-dependent glucuronoxylan methyltransferase (GXMT1) that specifically methylates O-4 of the GlcA substituents of GX. This OMT is a member of a family of proteins that contain a Domain of Unknown Function 579 (DUF579), which includes four phylogenetic clades. The Clade I in Arabidopsis contains AtGXMT1 and other two uncharacterized proteins that share high sequence similarity with GXMT1. To investigate if these two proteins are glucuronoxylan methyltransferases, we isolated and characterized two homozygous T-DNA insertion lines (SALK_{—}050883, which we named gxmt2-1 and SALK_{—}084669, which we named gxmt3-1) in which At1g09610 and At4g09990, respectively, are disrupted. RT-PCR analysis was used to confirm that the transcript was absent in these lines using primers to the full length coding sequence. The gxmt2-1 and gxmt3-1 plants were morphologically indistinguishable from wild type plants.

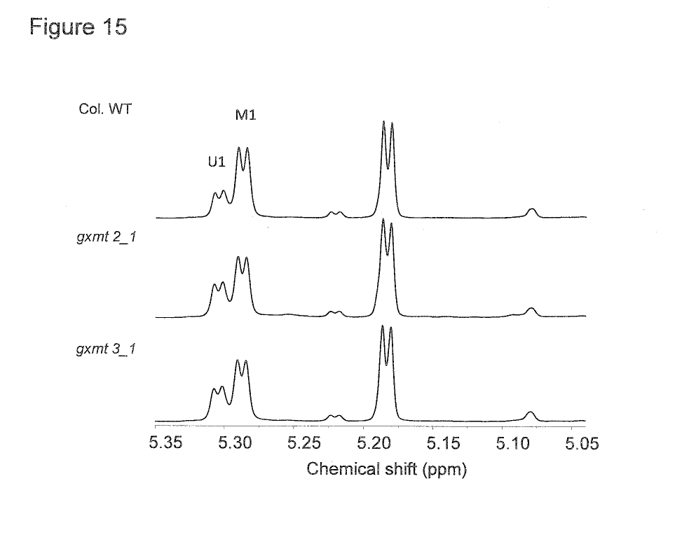

In order to determine if the GXMT2 and GXMT3 proteins are involved in O-methylation of GX, mature inflorescence stems of wild-type, gxmt2-1, gxmt3-1 and gxmt1-1 gxmt2-1 plants were sequentially extracted to obtain fractions enriched in hemicelluloses. The GX was extracted with 1N KOH, hydrolyzed with a β-endoxylanase, and the resulting GX oligosaccharides were analyzed by ¹H-NMR spectroscopy as described in Examples 1 and 2. The NMR analysis showed the degree of GlcA O-methylation of the GX synthesized by gxmt2-1 and gxmt3-1 was decreased by 12% and 9%, respectively, compared to wild type (FIG. 15, Table 1). These results, together with the amino acid sequence analysis, indicate that AtGXMT2 and AtGXMT3 are glucuronoxylan methyltransferases involved in xylan synthesis in Arabidopsis and that the GXMT2 and GXMT3 genes are good targets to manipulate to obtain biomass with reduced GX methylation.

TABLE 1
Reduction in O-methylation of GlcA in GX
isolated from gxmt2-1 and gxmt3-1 plants
relative to the degree of the wild-type.

| | Reduction of methylation |
|---|---|
| Plant | (% compared to WT) |
| gxmt 2-1 | 12 |
| gxmt 3-1 | 9 |

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1-91. (canceled)

92. A transgenic plant comprising decreased GXMT activity compared to a control plant, wherein the transgenic plant is not plant line SALK_{—}018081 or SALK_{—}087114.

93. The transgenic plant of claim 92 wherein the transgenic plant is a dicot plant.

94. The transgenic plant of claim 92 wherein the transgenic plant is a monocot plant.

95. The transgenic plant of claim 92 wherein the transgenic plant is a woody plant.

96. The transgenic plant of claim 97 wherein the transgenic plant is a member of the genus Populus.

97. The transgenic plant of claim 92 wherein the transgenic plant is switchgrass.

98. The transgenic plant of claim 92 wherein the transgenic plant comprises a phenotype of decreased recalcitrance.

99. A part of the transgenic plant of claim 92 wherein the part is chosen from a leaf, a stem, a flower, an ovary, a fruit, a seed, and a callus.

100. The progeny of the transgenic plant of claim 92.

101. The progeny of claim 100 wherein the progeny is a hybrid plant.

102. Plant material from the transgenic plant of claim 92.

103. A pulp from the transgenic plant of claim 92.

104. A method for using the plant of claim 92 comprising exposing biomass obtained from the plant to conditions suitable for the production of a metabolic product.

105. The method of claim 104 wherein the exposing comprises contacting the biomass with a microbe.

106. A transgenic plant comprising decreased expression of a coding region encoding a GXMT polypeptide compared to a control plant, wherein the transgenic plant is not plant line SALK_{—}018081 or SALK_{—}087114.

107. The transgenic plant of claim 106 wherein the transgenic plant is a dicot plant.

108. The transgenic plant of claim 106 wherein the transgenic plant is a monocot plant.

109. The transgenic plant of claim 106 wherein the transgenic plant is a woody plant.

110. The transgenic plant of claim 109 wherein the transgenic plant is a member of the genus Populus.

111. The transgenic plant of claim 106 wherein the transgenic plant is switchgrass.

112. The transgenic plant of claim 106 wherein the GXMT polypeptide is selected from a polypeptide having at least 80% identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:28, SEQ ID NO:29, SEQ ID NO:30, or SEQ ID NO:31.

113. The transgenic plant of claim 106 wherein the transgenic plant comprises a phenotype of decreased recalcitrance.

114. A part of the transgenic plant of claim 106 wherein the part is chosen from a leaf, a stem, a flower, an ovary, a fruit, a seed, and a callus.

115. The progeny of the transgenic plant of claim 106.

116. The progeny of claim 115 wherein the progeny is a hybrid plant.

117-120. (canceled)


SEQUENCE LISTING (53 sequences)

SEQ ID NO: 1 - protein, 298 aa, Zea mays
MSSPTHARKAIHLASLRARLAQQGKAGVALRLLLAAALAGFLLVFAARTLVTSPVPASPSYSYSSEPQPHPLPLPVAEALVHYATSNATPQQTAEEIGVALRVLQRRAPCNFLVFGLGLDSPMWAALNHGGRTVFLEEDAAWIGSVRGRHPALESHHVAYDTALADADALLGLRAHPACVAQPDLAAAAAASCRLALRGLPPVFAGLDWDLVMVDAPTGWTPRAPGRMGAIYTAGMAARARRPGAGPTHVFVHDVDRPVEDAFSRAFLCEGYLAEQVGRIRHFVIPSHRDKDGTPFCP

SEQ ID NO: 2 - protein, 298 aa, Medicago truncatula
MRSKTQLSSNLKLVIISLVFLFLFIMLFKSSIFSFTQPLTTTSNTNNDSKISSQETEKTTKSECPSLPITPTCTKNPPSLANALIHYATTKITPQQTIQEISVSAKVLQKKSPCNFLVFGLGHDSLMWTSLNYGGRTVFLEEDKTWIEQIQSKFPTLESYHVVYDTKVHQSDELMKIGMEQEDCKKVSDPRFSKCGLALKGFPSEIYDVEWDVIMVDAPTGYFDGAPGRMSAIYTAGLIARNRENGDTDVFVHDVDRKVEDQFSKAFLCEGYLREQEGRIRHFNIPSHRARLGRPYCP

SEQ ID NO: 3 - protein, 303 aa, Sorghum bicolor
MSSPMHVRKAIHFASLRAKLAQQGKAGLALRLLLAAALAGFLLVFAARTLLVSSPAAATSSLAECAAKGTAAATAGLPLAVMEALVHYTTSNVTPQQTADEIGVSLRVLQRRAPCNFLVFGLGHDSPMWAALNHGGRTVFLEEDASWIASVRATHPSLESYHVAYDTVLTDADALLQLRDHPACVAQPDLASAADASCRLALRGLPPVFHELEWDLIMVDAPTGWTPEAPGRMGAIYTAGMAARARRPGDGPTDVFVHDVDRPVEDAFSKAFLCEGYLAEQVGRIRHFVIPSHREKDGTPFCP

SEQ ID NO: 4 - protein, 295 aa, Oryza sativa
MSSPMHVRKAIHFVSMKAKLQSFGGLRLLLVGCLAALLLLFAVRTLSFTTSSATATAAREAAEAGCGKLPAAVAQAMVHYATANVTPQQTAAEIGVSLRVLQLRAPCNFLVFGLGLDSAMWAALNHGGRTVFLEEDASWIASVKAGHPGLESYHVAYDTRVTDADELIALRHEPACTSQPDLAAAAAASCRLALRGLPPVFHEVEWDLIMVDAPTGWTPESPGRMGAIYTAGMAARARTPGAGATEVFVHDVDRHVEDTFSKAFLCDGYLVEQVGRIRRFVIPSHRDKDGTPFCP

SEQ ID NO: 5 - protein, 288 aa, Populus trichocarpa
MRPKSQQISNLKLLLLGVFLAFFLLFVLRSSMSSSQESSSPTAQPKTSDSMKEARATNCSQGCSKVPRFLTQALIHYTTSTITPQQTQKEISVSAKILEKKSPCNFLVFGLGHDSLMWSALNYGGRTVFLEEDEAWIAQIKRRFPMLESYHVTYDSKVNQANNLMEVGKGPECTAISDPKFSMCQLALKGLPSEVYEIEWDLIMVDAPTGYYEEAPGRMTAIYTAGMMARNRKEGETDVFVHDVNREVEDKFSKALLCEGYMKKQVGRLRHFIIPSHRDALDRPFCPE

SEQ ID NO: 6 - protein, 306 aa, Glycine max
MRSKTTQFTCTFKVALLSLAVFCLFILVFRSSIFSLSPQLTSQTNMSSSDTIVPVSPQETENAGTPSSSSCPNSPPLNPPCTKAPPSLANAIIHYATANVTPQQTLHEISVSARVLEKKSPCNFLVFGLGHDSLMWTSLNYGGRTVFLEEDKSWIDQIQEKVPSLESYHVMYDTQVHQAEELMKTGMKEDCQKVTDPRFSRCPLAHKGLPSEVYDIDWDVIMVDAPTGYFDEAPGRMSAIYTAGLIARNKERGQTDVFVHDVDRKVEDMFSKAFLCEGYFKEQEGRIRHFNIPSHRSRLWRPFCPE

SEQ ID NO: 7 - protein, 168 aa, Zea mays
MSSLLQQGKAGVALRLLLAAALAGFLLVFATRTLVTSPVSASPSCSSEPLPRPVAEALVHYATSNTTPQQTSEEIGVALRVLQRHAPCNFMVFGLGLDSPMWAALNHGGRTVFLEEDAWIGSLRGRHPALESHHVAYDTALADADALLGLRAHPACIAQPDRRGERGE

SEQ ID NO: 8 - protein, 309 aa, Zea mays
MSSPTHVRKAIRFASLRAKIAQQGKTGLALRLLLAAALAGFLLVFAARTLVTPSPAPTSSSQAQEYHQDAAPECGKGNAGLPLAVAEALVHYTTSNVTPQQTADEIGVSLRVLQRRAPCNFLVFGLGLDSPMWAALNHGGRTVFLEEDASWIASVRSKHPGLESYHVAYDTVLTDADSLLGLRDHPACVAQPDLAAAAAASCRLALALKGLPPVFHELEWDLVMVDAPTGWTPQAPGRMAAIYTAGMAARARRPGDGPTDVFVHDVDRPVEDNFSKAFLCEGYLAEQVGRIRHFVIPSHREKDGTPFCP

SEQ ID NO: 9 - protein, 328 aa, Sorghum bicolor
MTSPMHARKAKLKTHLISAKAKLKHNVTPRRVVLLAAAATSAFLLLFTLRTLHSAASRHGAAAGAPAIAAHHADDHQELEHQQQHQQQECAAAKQVPASVAEALVHYATSNETPPRQTEAEAGAAARVLARRAPCSLLVFGLGPGSALWAALNHGGRTLFLEADADRIASARAARPAGIDLQAHPVAFQQEAIATPSDDLLVALRNSSDCAAAASSSPPKPLTPDHLEQSPCALAPRGLPAAFYEAEWDVIVVDAPVPGAVYTAGVAARARRPGTGETDVLVHGVDGAADESFTRAFLCEGYLKEEAGRLRHFSIPSHRDKDAMPFCP

SEQ ID NO: 10 - protein, 301 aa, Brachypodium distachyon
MSSPVHARKAIHTFSMKAAKLQSFLGLRVLLAAALAAFLLLFSARTLFPSSTNPPSSHALLHGGGESCGKLPAAVAETLVHYATTNTTPQQTAAEIGVSLRVLQRRSPCNFLVFGLGLDSPMWAALNHGGRTVFLEEDASWIESVRAAHSGLDLESYHVRYDTRLTDADDLLALRDHSDCVGADLDAGPESSSCKLALKGLPAVFREVEWDLIMVDAPTGWTPEAPGRMGAIYTAGMAARARRPGDGATDVFVHDVDRSVEDRFSKAFLCEAYLEEQVGRIRHFVVPSHREKPGTPFCPQN

SEQ ID NO: 11 - protein, 327 aa, Zea mays
MTSPMHARKAKLKTHFISAKAKLKHNVTPRRVVLLAAAATSAFLLLFTLRTLHSAASRSARASAAATVAAAIASTSPASVAADHADQLEQHHHQECAKVPSSVAEALVHYATSNETPHQTVAEAGAAARVLARRAPCSLLVLGLGPDSALWAALNHGGRTLFLEADAGRIASARAAHSAGIDLQAHPVAFQQEAMTPSDDLLALRNSSDCAASPPKPLTVDHLEQSPCTLAPRGLPETFYEAEWDVIMVNAPVPGAIYTAAVAARARRPGTGETDVLVHGVDDTTEESFARAFLCEPYLKEEASRLRHFSIPSHMDKDAMPFCPLIR

SEQ ID NO: 12 - protein, 296 aa, Mimulus guttatus
MRPKTKNPFNLKLLIIPTTLVFFLIFILRSNLSPSSNITTSPRSNIRQSKSLPKTKAEMKCPKNYCGKIPQSISEALIHYSTSTITPQQTLEEISITSRILEEYSPCNFLVFGLGHDSLMWHSLNHGGRTVFLEEDESWIEQIKRRFPMLESYRASYGTKVSQANDLMHVGKGIECTAVSDPRYSVCQLALKGLPSEVYEVEWDLIMVDAPTGYYEEAPGRMAAIYTAGMMARNRRRRGGGGGGGTHVFVHDVNREVEDKFSMEFLCEGYLKKQEGRLRHFVVPSHRDDLERPFCP

SEQ ID NO: 13 - protein, 316 aa, Oryza sativa
MASPMHARRAKLKSQLVSAKAKLKHHVTPRRLLLLSAAAASAFLLLLTLRTLSAAAANTSSPAPVVVHRSQQQQRDDQCDRVPAGVAEALVHYATSNATAWGRGRRRSAEEVAATARAVSRRAPCNLLVFGLGHGAALWAALNHGGRTVFLEEDDALVSGASPASLAIEAYRVAYLASAADADELLALRDSEHCTGAAATQLSPGHFDRSPCKLAVRGLPAAFYEAEWDVIVVDAHAPPPPTTTAMMGAIYTAAVAARARRPAAETETDVVVHDVDKPVQDRFSTAFLCGGYLKEGVGNLRRFAIPSHKEGMPFCP

SEQ ID NO: 14 - protein, 291 aa, Morchella esculenta
MRPKSQPIPILKLLLFGVFLAFFLLFLLRSNISSSKENPSPVSRTFQSKTPNHEEANATNCSPTSGKIPRSLAQALIHYTTSTITPQQTLKEISVTAKILEKKSPCNFLVFGLGHDSLMWSSLNYGGRTIFLEEDEAWISQIRRRFPMLESYHVTYDSKVNQANNLMEVGRGPECTAISDPKFSMCQLALKGLPSEVYEIKWDLIMVDAPTGYYEEAPGRMTAIYTAGMMARNKEEGEETEVFVHDVNREVEETFSKVFLCEGYMKKQEGRLRHFTIPSHRGALERPFCPE

SEQ ID NO: 15 - protein, 319 aa, Brachypodium distachyon
MTSPPLARKARLKTHLVSAKVKLRQHVTLRRALLLAAASAASFLFLLTLRTLLSSSHARIQTPATQLQLRGQHCAKLPAPVAEALVYYATTSANNATSSQQQTAAELAVTARALARCRAPCNVLVFGGLGGTESALWAALNHGQGGRTVFLEEDASLISEISSRHPGLGIEPHQVAYQTTLADADELLALRDSPDCKKTQNRPLSPDEFESSKCKLAMRGLPAAFYETEWDVIIVDAPPGWVPEAPGRIGGAIYMAGMAARARRWPGKTEVIVHDVDRAVEDGLSMAFLCRGYLEEEVGRVRRFAVPSHREKDVMPFCP

SEQ ID NO: 16 - protein, 352 aa, Populus trichocarpa
KQSLPSTLKLSTSQLKPTEIYSLHAASKTQTNNIQSPLLFHSKPEKCKDLFLSFSRSIPKIKSNMRPKGQQITNLKMLLFGVFLAFFVLFVLRSGMPSSQESPSPTSQPGVLNPMKEARETNCSQACTQIPRSLTQALVHYTTSTITPQQTRKEISVSAKVLEKKSPCNFLVFGLGHDSLMWSALNYGGRTVFLEEDEAWIAQIKRRFPMLESHHVTYDSKVNEAANLMEVGKGPECTAVGDPKFSMCQLAMKGLPSEVYEIEWDLIMVDAPTGYHDEAPGRMTAIFTAGMMARNRKEGETDVFVHDVNREVEDKFSKSFLCEGYMKKQVGRLRHFTIPSHRDDLDRPFCPE

SEQ ID NO: 17 - protein, 333 aa, Glycine max
MQQQALQSLLDSQFRQVGRQVFCLHPSILHAYSLGVQASIKHNRQFHARRRSSMRTKAHQPGLNLKLLIVAVLVAITLLFVLIRSSNTKGVKQQEALLVKSDGSCNKMKIPGSVSEALIHYSTSSITPQQTVKEISVTARVLEKKSPCNFLVFGLGHDSLMWNALNHGGRTIFLEEDESWIQQMRRRFPMLEAYQVNYESKVNEAESMMEVGRGGECRAVGDPRYSICPLALKGLPSVVYETKWDLIMVDAPTGYYENAPGRMTAIYTAGMMARNRESGETEVFVHDVNRVVEDQFSRAFLCQKYMKKQEGRLRHFTIPSYRDHHQDMPFCPP

SEQ ID NO: 18 - protein, 301 aa, Populus trichocarpa
MRSKNQSPINIKIIILGLFFLLFLLVIAWSSYSSPRPNSSPKAENDDLSDSSNLSTDEEEPKPSTAACPSLPLTPTCTKIPPSLANALVHYVTTNITPQQTLKEISVSLRVLEKKSPCNFLVFGLGHDSLMWTSLNHGGRTVFLEEDKAWIEQIREKLPTLESYHVTYDTRVHQADGLMETGMGDECKVVGDPRFSKCQLALKGFPGDIYDVEWDLIMVDAPTGYHDEAPGRMNAIYTAGLMARNRENGVTDVFVHDVDRVVEDKFSKAFLCEGYMTEQEGRLRHFIIPTHRTSSGKPFCP

SEQ ID NO: 19 - protein, 350 aa, Zea mays
MAPRTPARPSTWPPCARLAQQGKVGVALRLLLAAVLAGFLLVFVARTLVTSPVPASPSCSSEPQPQPLSLPVAEALVHYATSNATPQQTTEEIGVALRVLQRHAPCNFLVFGLGLDSPMWAALNHGGRTMFLEEDAAWIGSVRGRHPALESHHVPYDTALADVDALLGLRAHLACVVQPDRRGERRERATTSSKATRRRPPHQLPPPAPKPSTPRRPLPRQEEDFEVDFEEFGVKAGDSELDSADEAMTFAAPGAHSSEVTTGSSQSPELSLSVTPFLAAIAVPLEEKTSTVDFVWAIPLSLLFSGDLQPSRRRCLYTMASVMLIFSAKVIDIHQIIPLVKAAGPDKMVC

SEQ ID NO: 20 - protein, 304 aa, Populus trichocarpa
MRPNKNQSPINIKIITLSLFFLLFLVLVAWSSFSSPRSNPSPKAENDDLTNPSSSLSTDEEDQPKPSTPACPSLPLTPTCTKTPPSLANALVHYVTTNITPQQTLKEISVTLRVLEKKSPCNFLVFGLGHDSLMWTSLNHGGRTVFLEEDKAWIEQITEKLPSLEAYHVTYDTRVHQADGLMETGMGDECKVVSDPRFSKCQLSLKGFPSDIYDMEWDLIMVDAPTGYHDEAPGRMTAIYTAGLMARNRENGETDVFVHDVDRVVEDKFSKAFLCEGYLTEQEGRLRHFIIPSHRTRSGRPFCP

SEQ ID NO: 21 - protein, 282 aa, Arabidopsis thaliana
MRPKANQNHKLKVLLVFLLATLILIFIVRSTLTSSQEHQTPQETRSTRCSGACNKLPRSLAQALIHYSTSVITPQQTLKEIAVSSRVLGKKSPCNFLVFGLGHDSLMWSSLNYGGRTVFLEEDEAWIKQIKRRFPMLESYHVTYDSKVNQADNLIEVGKGPECTAIGDPRYSMCQLALKGLPAEIYETGWDLIMVDAPTGYYDEAPGRMTAIYTAGMMARNRKQGGETDVFVHDVNREIEDKFSKAFLCEGYMKKQEGRLRHFIIPSYRDGSESESNRPFCP

SEQ ID NO: 22 - protein, 297 aa, Arabidopsis thaliana
MRTKSPSSLNLKVIFIGSSILILIIIYLARSNISSSSSKPISKTNLSQEEEETQHKQEGCPTTQQCTKMPLSLSDALVHYVTSNVTPQQTFDEVSVSKRVLDKKSPCNFLVFGLGHDSLMWASLNHGGRTLFIEEDQAWIAIVTKKFPNLESYHVVYDTKVKDSDKLMELGRSEECRSVSDPRNSKCDLALKDFPADFYETKWDLIMVDAPTGYHEEAPGRMSAIYTAGLLARNREDGETDVFVHDVNRPVEDEFSATFLCKGYMREQNGRLRHFTIPSHRARAGRPFCPVEVDRRR

SEQ ID NO: 23 - protein, 298 aa, Setaria italica
MSSPMHVRKAIHFASLRARFAQGKGGLALRLLLAAALAGFLLVFAARSLSSPSPSTSRRQEAAECGGEGKGLPLPVAEALVHYTTSNVTPQQTADEIGVSLRVLQRRAPCNFLVFGLGFDSPMWAALNHGGRTVFLEEDASWIANVRSKHPALESYHVTYDTVLTESDALLELRDHPACVAQPDLASAAEASCRLALKGLPPVFHDLQWDLIMVDAPTGWTPEAPGRMGAIYTAGMAARARRPGEGATDVFVHDVDRPVEDAFSKAFLCEGYLAEQVGRIRHFVIPSHREKEGTPFCP

SEQ ID NO: 24 - protein, 194 aa, Setaria italica
MALRPARPLPAARRRSTTKQLGKHKGNHEDGPNRKAYHVAYDDTKVGDADKLLELRGDPACVAQPDLAAAPCRLAPPGLPAAFYKAEWDVILVDAPTGYAPWSPGRMGAIYAAGMAARARRPGATTDVLVHDVDRPVEDRFSRAFLCEAYMVEQVGELRHFAVPSHRGRDAVPSHRGRDAVPPFCPHDEDTANA

SEQ ID NO: 25 - protein, 295 aa, Panicum virgatum
MHVRKAMHLASLRARLAQGKGGLALRLLLAAALAGFLLVFAARSLSSPPPSPAASRREECGGDKAGKGLPLAVAEALVHYTTSNTTPQQTADEIGVSLRVLQRRAPCNFLVFGLGFDSPMWAALNHGGRTVFLEEDASWIADVRSKHPSLESYHVAYDTVLTDADALLELRQHPACVAQPDLASAAGASCRLALRGLPPVFHELEWDLIMVDAPTGWTPQSPGRMGAIYTAGMAARARRPGDGPTDVFVHDVDRPVEDAFSRAFLCEGYLAEQVGRIRHFVIPSHREKDGTPFCP

SEQ ID NO: 26 - protein, 303 aa, Panicum virgatum
MSSSSPMHARKAIHLASLRARLAQGKGGLALRLLLAAALAGFLLVFAARSLSSPSPPPSTSASRRQECGGGGGDKGLPLAVAEALVHYTTSNTTPQQTADEIGVSLRVLQRRAPCNFLVFGLGFDSPMWAALNHGGRTVFLEEDASWIASVRSKHPSLESYHVTYDTVLTDADALLELRDHPACVAQPDLASAAEASCRLALRGLPLVFHELEWDLIMVDAPTGWTPQSPGRMGAIYTAGMAARARRPGDGPTDVFVHDVDRPVEDAFSKAFLCEGYLAEQVGRIRHFVIPSHREKDGTPFCP

SEQ ID NO: 27 - protein, 307 aa, Eucalyptus spp.
MGSSNGNTSKTHFAINAKIILLSFLFLFLLLLIVRSTFPSSPQLSPVPSSTTELTVQAKAQDTAPAVDCPTTAAAAAAATGTCTKTPPSLAQALVHYATTNITPQQTYDEISVSLRVLAKKSPCNFLVFGLGHDSLMWTSLNHGGRTVFLEEDKAWIHQITQKLPTLEAFHVTYDTKVTQADNLMKLREEEECKAVTDPRSSKCELAHKGFPSEVYEVEWDLIMVDAPTGWHDEAPGRMSAIYTAGLMARNRESGETDVFVHDVDRVIEDRFSKAFLCEGYLTEQVGRLRHFTVPSHRARSGRPFCP

SEQ ID NO: 28 - protein, 256 aa, Eucalyptus spp.
MKPKATYPINHKFLLLGIFLAFLVIFILRSSFSSSNEVSSSVSQNSSLKASSEAKASDCSPTCSKIPNPLAQALIHYTTSTITPQQTLKEISVTARVLKKKSPCNFLVFGLGHDSLMWSSLNYGGRTIFLEEDEAWIEQIKKRFPMLESYHVSYDSKGLPDEVYERQWDLIMVDAPTGYYEEAPGRMTAIYTAGMMARNRKEGETDVFVHDVNRSVEDKFSKEFLCEGYMKKQENRLRHFIIPSHTGESSMPFCPV

SEQ ID NO: 29 - DNA, 967 bp, Zea Mays
cggaggaggagagccatgtccagccccacgcacgcccgcaaggccatccacctggcctccctgcgagccaggctcgcgcagcagggcaaggccggcgtggcgctgcgcctgctcctcgccgccgcgctcgccggcttcctcctcgtcttcgccgcgcgcaccctggtcacgtcgcctgtcccggcatcgccctcgtactcatactcgtcggagccgcagccgcatccgctgccgctgcccgtggcggaggcgctggtgcactacgccacgtccaacgcgacgccgcagcagacggcggaggagatcggggtggcgctgcgcgtgctgcagcggcgcgcgccctgcaacttcctggtgttcggtctgggcctcgacagccccatgtgggcggcgctgaaccacggggggcggaccgtgttcctggaggaggacgcggcgtggatcggcagcgtgcggggcaggcacccggcgctggagtcccaccacgtggcctacgacacggcgctggccgacgccgacgcgctgctagggctccgcgcccacccggcctgcgtcgcgcagcccgacctcgcggcggccgccgccgcgtcctgccgcctcgcgctccggggcctcccgcccgtcttcgccggcctcgactgggacctcgtcatggtggacgcgcccacggggtggacgccccgggcgcccggccgcatgggcgccatctacaccgccggcatggcggcgcgcgcgcgcaggccaggggccggccccacccacgtcttcgtccacgacgtcgaccgccccgtcgaggacgccttctccagggcgttcctgtgcgagggctacctcgccgaacaggtcggacggatcaggcacttcgtcatcccgtcacaccgcgacaaggacggcacgccattctgcccttgacaagagtttgttctgctttggtttggatcccacggtgccaattgccaaactacta

SEQ ID NO: 30 - DNA, 1807 bp, Oryza sativa
ctgcaattaatcaatctgttaatctgttattagtagttactactaacaattatagagtatattaattaatttaagctaaaaaaatggggcccacaaagcagaggaagagaacccatccatccaaccattcataaaatctatctagaattccaacttaacaccaaatctattggtctcttgtcctcatcaccaaaccccatcttcatcgtcgtcatcttcaacccccatccccatatcttcctttgcctctccttcaagcatcatcaccaagtaaaaagagaaaaagaacccataaatttgaagaattttagcttcattcaagaactcgatcgaggagggccaagaaaggtgtgagaccttgatttggtcgccatgtcgagccccatgcatgttcgcaaggcgatccacttcgtctccatgaaggccaagctccagagcttcggcggcctccgcctcctcctcgtcggctgcctcgccgcgctgctgctgctgttcgccgtcaggacgctgtcgttcacgacgtcgtcggcgacggcgacggcggcgagggaggcggcggaggcgggctgcgggaagctcccggcggcggtggcgcaggcgatggtgcactacgcgacggcgaacgtgacgccgcagcagacggcggcggagatcggggtgtcgctgcgggtgctgcagctgcgggcgccgtgcaacttcctggtgttcggcctgggcctcgacagcgccatgtgggcggcgctcaaccacggcggccgcaccgtgttcctcgaggaggacgcgtcctggatcgcctccgtcaaggcgggccacccggggctcgagtcctaccacgtcgcctacgacacccgcgtcaccgacgccgatgagctcatcgccctccgccacgagccggcctgcacgtcccagccggacctcgccgccgccgccgccgcgtcgtgccgcctcgccctccgcgggctcccgccggtgttccacgaggtggagtgggacctcatcatggtggacgcgccgacggggtggacgccggagtcgccggggaggatgggggcgatctacaccgcgggcatggcggcgcgcgccaggacgcccggggcgggcgccaccgaggtgttcgtccacgacgtcgaccgccacgtcgaggacaccttctccaaggcgttcctctgcgacggctacctcgtcgagcaggtggggcgcatccgccgcttcgtcatccccagccaccgcgacaaggacggcacgccgttctgcccttgatgagagatgagcttcgccttcgccttcgccgcgcacatggacaccgtgatgacaccattgccatcgtcgtcttcttcatcttcttcatcttcttcttcacatatgacaccaccattgctgccgtcatcgtcttcttgaattcacatgtcttttgtgatgatcaaggataagtaggatgatgagtgcacctttgggatcatgcaccaatggaggcggtggcgcgatgcgtgtgagccgtcggatcgagaacggacggttcggatcggaccgtggagatattgttattatatatagtttcttttttttttcaaatgcagctttttctttgttgttttttttatggacggtgggtgagatggatatgcaggcgaattataattcttggctatttttcatagattatcgttgtgtttctttttaattcataattgagatatcatcttcagtgggtgactgatgagtggtgccatttgagattcctatctactgaagcaataatgctaagatagaaacttacataatttgttgagaaatgcagttgattttt

SEQ ID NO: 31 - DNA, 1173 bp, Populus trichocarpa
tattttttattcgatatctgaaaggagaagaaaccttttttgagtttctcctactctatcccagagatcaatccaaaagggctgggaaatatgaggcctaaaagccagcaaatctccaaccttaagctgctcctccttggtgtcttccttgctttcttcctgctttttgtgttacgatcaagcatgtcatcttcccaggaaagttcatctcccaccgctcaaccaaaaacctcagattccatgaaagaagctagagcaacaaattgctcacagggatgtagtaaggtcccacgcttcctaacccaagctctcattcactacacaacctcaaccatcaccccacagcaaacacagaaagaaatatcagtgagtgccaaaattttagaaaagaagtctccatgtaatttcttggttttcggcctcggtcatgatagccttatgtggagtgcactcaactacggtggacgaacagttttccttgaagaagatgaagcttggattgcgcaaattaaacgccggtttcctatgttagagtcttatcacgtcacatatgacagcaaggtgaatcaagctaacaatctcatggaggtaggcaaggggcctgagtgcacagcaattagtgatccaaagttctctatgtgccagcttgccttgaaaggtttgcctagtgaagtttatgagatagaatgggacttgatcatggttgatgcaccaacagggtactatgaagaggcaccagggagaatgactgccatatatactgccggaatgatggcaagaaacaggaaagaaggagagaccgatgtttttgtgcatgatgtgaacagagaggtggaagataagttctccaaggcacttctttgtgaaggatatatgaagaaacaagtagggaggttaaggcactttatcattcctagccacagggatgccttggacagacctttttgcccagagtagattcttttcttttcttttctttttaatctcttttcctatgcaatttttgtgcatctaagaacaaacttggtatcattttcaaagaacatgcatggcttctgttgaatgttgagtaggcagctaaaaccaaactggaagtttctctccattgtgattgaattgatatttgataaatggcagagcataataagaaggtacaaaagattgaagtttctc

SEQ ID NO: 32 - DNA, 1402 bp, Zea Mays
caaatttctcctcccctccctagtccctaccaaattcgccaccatccccatctatattatccagtcctctgctcatccatacctttgctttgcctctccttcacttcacttcaactccatcgcccgttgcagcggcggcggcggcaatcatatcacccagacgcggacgccatagtagattagattgatctctccgtggggaggaagaggagcaagagccatgtccagccccacgcacgtccgcaaggccatccgcttcgcctccctgcgggcaaagatcgcgcagcagggtaagacaggcctggcgctgcgcctgctcctcgccgccgcactcgccggcttcctcctcgtgttcgccgcgcgcaccctggtcacgccgtcccctgccccgacctcgtcgtcccaggcgcaggaatatcaccaggacgccgcgccggagtgcggcaaggggaacgcgggcctgccgctggcggtggcggaggcgctggtgcactacaccacctcgaacgtgacgccgcagcagaccgcggacgagatcggggtgtcgctgcgcgtgctgcagcgccgcgcgccctgcaacttcctcgtcttcgggctgggcctcgacagccctatgtgggcggcgctgaaccacggcggccgcaccgtgttcctggaggaggacgcgtcgtggatcgccagcgtgcgttccaagcacccggggctcgagtcctaccacgtcgcctacgacaccgtgctcaccgacgccgactcgctcctggggctccgcgaccacccggcctgcgtcgcgcagccagacctcgccgccgccgccgccgcgtcctgccgcctcgcgctcgccctcaagggcctcccgcccgtcttccacgaactggagtgggacctcgtcatggtcgacgcgcccacggggtggacgccgcaggcgcccggacggatggccgccatctacaccgccggcatggcggcgcgcgcgcgcaggcccggggacggacccaccgacgtcttcgtgcacgacgtcgaccgacccgtcgaggacaacttctccaaggcgttcctgtgcgagggatacctcgccgagcaggtcggccggatcaggcacttcgtcatcccgtcccaccgtgagaaggacggcacaccattctgcccttgacaatctttattttgattctttgagagtttgagtgagagccatccatccataatccatctctctttttttttttgcttttcctgcgctctgccttggctttatttacatacatatatatgagacgaaaaaacactacagcatgcatatgcagcagccgtcagcaactggagctccttcatcattctttctgtactactactactaccgttgcagtattactcctgctctagagctgttgccaaaaaaaattaa

SEQ ID NO: 33 - DNA, 1356 bp, Oryza sativa
aaatcgcaatgcatcatatcattgcataatcacaacaacacaccactaatctaaagcaaagtgaaaaagaaaaggagacactagcaacttagcttagcttagctagtgcaagaagaagaggtgcaatggcgagccccatgcacgcgcggagggcgaagctgaagagccagctggtgtcggccaaggccaagctcaagcaccatgtcacgccgcgccgcctcctcctcctctccgccgccgccgcctccgccttcctcctcctcctcaccctccgcaccctctccgccgccgccgccaacacctcctcgccggcgcccgtcgtcgtccaccgctcgcagcagcagcagcgggacgatcagtgtgaccgtgtcccggcgggcgtggcggaggcgctggtccactacgcgacgtcgaacgcgacggcgtgggggcgggggaggaggaggtccgccgaggaggtggcggcgacggcgcgggcggtgtcgcggcgggcgccgtgcaacctgctcgtgttcgggctcggccacggcgccgcgctctgggcggcgctcaaccacggcggccgcacggtgttcctcgaggaggacgacgcgctggtgtccggcgccagcccggccagcctcgccatcgaggcctaccgcgtcgcctacctcgccagcgccgccgacgccgacgagctcctcgccctccgcgactccgagcactgcaccggcgccgccgccacccagctctcgccgggccacttcgaccgctcgccgtgcaagctcgccgtgcgcggcctccccgcggcgttctacgaggcggagtgggacgtgatcgtggtcgacgcgcacgcgccgccgccgccgacgacgacggcgatgatgggcgcgatatacaccgcggcggtggcggcgcgggcgcggcggccggcggcggagacggagacggacgtggtggtgcacgacgtggacaagccggtgcaggacaggttctcgacggcgttcctgtgcggcggctacctcaaggagggggttggcaacctcaggcgattcgccatccctagccacaaggaaggaatgccattttgcccttgaaatattttattatcattgggtaaatatataccattttaccagtaattaaacacatattagtagcatatgcatatttaaatatttgtataggatttatgttagaggaaacaataatatatttgtagatttcttcttctgcttctttgtgttcctttttgttgccatggtttttctcaaatctcaactcttgttgataacttgaaatggggacaacatttcggaatttcaaattcactgtcgaatttgaagtaactatgagttaatgacatttttatgatct

SEQ ID NO: 34 - protein, 147 aa, Medicago sativa
MKELREVTAKHPWNIMTTSADEGQFLSMLLKLINAKNTMEIGVYTGYSLLATALAIPEDGKILAMDINKENYELGLPVIKKAGVDHKIDFREGPALPVLDEMIKDEKNHGSYDFIFVDADKDNYLNYHKRLIDLVKVGGVIGYDNTL

SEQ ID NO: 35 - DNA, 1307 bp, Populus trichocarpa
atactatctctgaattgttctttgcattgttaatttttgagatcataaattatatccacatgcaaaataataataaaaaagcatcttatatttcttttttatcttatatacctctcaaagaaagcaaaaacaaataatttcttgtctcctaggagcaaccacaagccccccactttctttcattaatatggactaaattcaacctctgttcatcatccctttcccccactctctcgccctccgaagaaaaaccaaacgcctgatcaagaaccatgaggtccaaaaaccaatctcccattaacatcaagatcatcatcctcggtttattctttctcctcttccttttggtgattgcttggtcaagttactcgtctcccagaccaaattcatcccccaaagcagaaaatgatgatctatcagattcatcaaatctgtccactgatgaagaagaaccaaaaccatcaacagcagcctgtccttctcttccattgacgccgacatgcaccaagatcccaccttctttagccaacgctcttgtccattatgtgacaacaaacatcaccccacaacaaaccctcaaggaaatctcagtctcattaagggtcctcgaaaaaaagtcaccttgcaacttcctagtctttggccttggccatgacagtctaatgtggacatctctaaatcacggcggccgcactgttttcctggaagaggacaaggcctggattgagcagatcagagagaaactgcccaccttggagtcctatcatgtgacatacgacaccagagtgcatcaggcagatgggctcatggagacaggaatgggagatgaatgcaaagtagtcggtgatccaagattctccaagtgtcagcttgcccttaaagggttccctggtgatatctatgatgtggaatgggacttgatcatggtggatgcacctactgggtaccatgatgaggctccagggagaatgaatgcaatctacacagctggattgatggctagaaacagagaaaatggagtgactgatgtgtttgtgcatgatgttgatagagttgtggaggataaattctccaaggcttttctttgtgaaggatacatgactgagcaagaagggaggttaaggcacttcattattcctactcatagaactagttcgggaaaacctttttgtccttagaggtttctcatctctcttttctgcttaaatttgctgttattaattgtttatcttgatctttgtttcttgtcatataaaatataaattaccgggcaaaggtttttttttatattgttactaattaatttc

SEQ ID NO: 36 - DNA, 1121 bp, Populus trichocarpa
ctctctctctctctctctctatccaaagaaaaaccagagacgtcaagaaccatgagacccaacaaaaaccaatctcccattaacatcaagatcatcaccctcagtttattctttcttctcttccttgtgctggtagcttggtcaagcttctcatctccgagatcaaatccatcccccaaagcagaaaatgatgacctaacaaacccatcatcaagtctgtccactgatgaagaagatcagcccaaaccatcaacaccagcctgcccttctcttcccctgacaccaacatgcaccaaaaccccaccttctttagccaatgctcttgtccattacgttacaacgaacatcaccccacaacaaaccctcaaagaaatctcggtcacattaagggtcctggagaaaaagtcaccttgcaatttcctagtctttggtcttggtcatgacagtctaatgtggacatctctaaatcatggtggccgaactgttttcctggaagaagacaaggcttggattgagcaaatcacagagaaactgccctctttagaggcctaccatgtgacgtacgacaccagagtgcatcaggcagatgggctcatggagacaggaatgggagatgaatgcaaagttgtaagcgaccccagattctccaagtgccagctttctcttaaagggttccctagtgatatttatgacatggaatgggacttgatcatggtggatgcacccactgggtatcatgacgaggcaccagggaggatgactgcaatctacacggctggattgatggctaggaacagagagaatggagagactgatgtgtttgtgcatgatgttgatagagttgtggaggataaattctccaaggcttttctttgtgaaggatacttgactgagcaggaagggaggttaaggcatttcatcattcctagtcatagaactcgttcgggcagacctttttgtccttagagttcctcatctctcttttctgcttagatttgttgttattgtttctcctgatcttgtttcttgtcatataaaatataaatttctgggcaaagggttttttcttttcatatatgtattttatattgttcccaattaattaatttctcttccctcgg

SEQ ID NO: 37 - DNA, 888 bp, Panicum virgatum
atgcacgtccgcaaggcgatgcacctggcgtccctgcgcgccaggctcgcgcaggggaagggcggcctcgcgctgcgcctcctcctcgccgcggcgctcgccggcttcctcctcgtcttcgccgcgcgctcgctctcctccccgccgccgtcgccggcagcgtcccggcgggaggagtgcggcggcgacaaggcggggaaggggctgccgctggcggtggcggaggcgctggtgcactacacgacctccaacacgacgccgcagcagaccgccgacgagatcggggtgtcgctccgggtgctgcagcgccgggcgccctgcaacttcctcgtcttcgggctcgggttcgacagccccatgtgggcggcgctcaaccacggcggccggacggtgttcctggaggaggacgcgtcgtggatcgccgacgtccgctccaagcacccgtccctcgagtcctaccacgtcgcctacgacaccgtcctcaccgacgccgacgcgctcctcgagctccgccagcacccggcctgcgtcgcgcagccggacctcgcctccgccgccggggcctcctgccgcctcgccctgcgcggcctgccgcccgtgttccacgagctcgagtgggacctcatcatggtggacgcgccgacggggtggacgccgcagtcccccggccggatgggcgccatctacaccgccggcatggcggcgcgcgcgcggcggcccggcgacggccccaccgacgtgttcgtgcacgacgtcgaccgccccgtcgaggacgccttctctagggccttcctctgcgagggctacctcgccgagcaggtcggccggatcaggcacttcgtcatcccgtcgcaccgggagaaggacggcacgccattctgcccttga

SEQ ID NO: 38 - DNA, 912 bp, Panicum virgatum
atgtcgagctcgagccccatgcacgcccgcaaggcgatccacttggcgtccctgcgcgccaggctcgcgcaggggaagggcggcctcgcgctgcgcctcctcctcgccgcggcgctcgccggcttcctcctcgtcttcgccgcgcgctcgctctcctccccctccccgccgccgtcgacgtccgcgtcccggcggcaggagtgcggcggcggcggcggcgacaaggggctgccgctggcggtggcggaggcgctggtgcactacacgacctccaacacgacgccgcagcagaccgccgacgagatcggggtgtcgctccgggtgctgcagcgccgggcgccctgcaacttcctcgtcttcgggctcgggttcgacagccccatgtgggcggcgctcaaccacggcggccggacggtgttcctggaggaggacgcgtcgtggatcgccagcgtccgctccaagcacccgtccctcgagtcctaccacgtcacctacgacaccgtcctcaccgacgccgacgcgctcctcgagctccgcgaccacccggcctgcgtcgcgcagccggacctcgcctccgccgccgaggcctcctgccgcctcgccctgcgcggcctgccgctcgtgttccacgagctggagtgggacctcatcatggtggacgcgccgacggggtggacgccgcagtccccgggccggatgggcgccatctacaccgccggcatggcggcgcgcgcgcggcggcccggcgacggccccaccgacgtgttcgtgcacgacgtcgaccgccccgtcgaggacgccttctccaaggccttcctctgcgagggctacctcgccgagcaggtcggccggatcaggcacttcgtcatcccgtctcaccgggagaaggacggcacgccattctgcccttga

SEQ ID NO: 39 - DNA, 1709 bp, Eucalyptus spp.
gtgaaatacaacctatttgttcgccgcgaatttggatagttaatcggccttccaaatggtaattatctttatttatggtgcgaggtgccggtcggcggttgtgcaatgatgtaggggttattgtgtacgtactatacacgacatccgatgctttcggagggtacagccgctggaccaccaatcctatgaggcgcagacacgtgtgcccgtattattgccgtggttcggacgggtcattactgttcagttcgcataattccaactcaaacgccgttttccccgaatcaacttacctttagtttagtcctgcggaaggaaagaacagaacagacagaaaccataggaaaaacagggaagcgggttcacgctctttataggtattcggtgagagaaagctagctctttcttcagacagactttcaagcaaaagaccagagcagtcctctggtctctcctctgtcaaacgcaccatcaagcgaattctcgagacagaatcgggtcgtcttcgcacatcggaaaatgggttccagcaacggcaacacgagcaagacccatttcgccatcaacgccaagatcatcctcctcagcttcctcttcctcttcctcctcctcctcatcgtccgcagcaccttcccttcctctccgcagctctcgcccgttccgtcgtccaccactgagctgacggtccaggcaaaggcacaggacactgcccccgccgtcgactgcccgaccacagccgccgccgccgccgccgccaccgggacgtgcaccaagacgccgccgtccttggcccaagccctagtccactacgccaccaccaacatcaccccgcaacagacgtacgacgagatctccgtctccctccgggtgctggccaagaagtcgccgtgcaacttcctcgtgttcggcctcggccacgacagcctcatgtggacctccctcaaccacggcggacgcaccgtcttcctggaggaggacaaggcctggatccaccagatcacccagaagctcccgacgctggaggctttccacgtcacctacgacaccaaagtcacccaggccgacaacctgatgaagctccgggaggaggaggagtgcaaggcggtgacggacccaaggtcgtccaagtgcgagctggcgcacaaggggttcccgagcgaggtgtacgaggtggagtgggacctgatcatggtggacgcgcccaccgggtggcacgacgaggcccccgggaggatgagcgccatctacacggcgggcctgatggcgcggaaccgggagagcggcgagaccgacgtgttcgtgcacgacgtcgaccgggtcatcgaggaccgcttctccaaggccttcctgtgcgagggctacctgacggagcaggtgggcaggttgcgccacttcaccgtgcccagccaccgggcccgctccggccgacccttttgcccctagaatctgctaatcttttctctttttcatttgcattttcctgggggtaattacggaaatctgccacgacggaaagaatttctttttctttttttcttttttttacttgtgaatccttttttatattatttgtaattttattttatattaccgggaggggaattcgttccctattttcactttaggacgtgcgcgcggccaggacacgttgacagagacttcggattcccattaataagttttattataaaatactcgaattctgggga

SEQ ID NO: 40 - DNA, 1308 bp, Eucalyptus spp.
attgcttcacattcgacagcgcagagagttctttcttatcgcttcctggatcaaaacgagggatagaactagaagcatgaagcccaaggccacttaccccatcaaccacaagttccttctcctaggcatcttcctcgcatttctcgttatcttcattttgagatcaagcttttcttcttccaatgaagtctcctcttccgtttcccaaaactcctccttgaaagcctcgtcagaagcaaaggcatccgactgctcgccgacttgttccaagatcccgaatccactagcacaagctctcatccactatacaacgtcgaccatcacccctcaacaaacactcaaggagatatcggtgactgctcgggtcctcaaaaagaaatcgccatgcaatttcctcgtgttcggactcggccacgacagtctcatgtggagctccctcaactatggcgggcgaactatcttcctagaagaagacgaggcgtggatagagcaaatcaaaaaaaggtttccgatgctggagtcataccacgtgagttatgatagcaagggcttgccagacgaagtttacgagagacaatgggaccttatcatggtcgatgcgccgacgggatactatgaggaggcaccggggaggatgacggcgatatacacagcagggatgatggctaggaataggaaggagggagagacggatgtgtttgtgcacgacgttaacaggtccgtagaagacaaattctccaaggaattcttgtgtgagggttacatgaaaaaacaggaaaacaggttgaggcacttcatcatccctagccacacaggcgagtccagcatgcccttctgtccagtctaaaattagaggccaaagccaaattttcggatttctttctcatggaatctggggttttcttgttcatggagcggccacagggccaccgacgcgggtctaggacagcgttttgagcttataaataagcatgtgtgctgtagtccttccttgtcataagcatcacttttctccccgcaatgtaaaaatacacttgggctatttcactcttcttatatcttccttcgatccacaaaccatgtctgcttaaaagccccgtcttgttcagggctaccatgcgttctctctttaggtaaccccaaagatcatgacaaggaaattcagtcagtctgccagaggatacgaaaaaagcctccagcgtccatgaactcaaagttgcattcgcgttcacatcgaaatcgttggcggtcgttttcatttaataagatagaggaccaagccgtctgttccacatcataatccgcaat

SEQ ID NO: 41 - DNA, 25 bp, artificial
  synthetic oligonucleotide primer
atgaggaccaaatctccatcttctc

SEQ ID NO: 42 - DNA, 19 bp, artificial
  synthetic oligonucleotide primer
acggcggcgatcaacttcc

SEQ ID NO: 43 - DNA, 31 bp, artificial
  synthetic oligonucleotide primer
gcgcggaattcaacaaatctctcccaagaag

SEQ ID NO: 44 - DNA, 29 bp, artificial
  synthetic oligonucleotide primer
gcgcgctcgagacggcggcgatcaacttc

SEQ ID NO: 45 - DNA, 21 bp, artificial
  synthetic oligonucleotide primer
tgcaactaccatgttggttcc

SEQ ID NO: 46 - DNA, 23 bp, artificial
  synthetic oligonucleotide primer
agtttcaccatcttcacggttac

SEQ ID NO: 47 - DNA, 19 bp, artificial
  synthetic oligonucleotide primer
attttgccgatttcggaac

SEQ ID NO: 48 - DNA, 25 bp, artificial
  synthetic oligonucleotide primer
atgaggaccaaatctccatcttctc

SEQ ID NO: 49 - DNA, 19 bp, artificial
  synthetic oligonucleotide primer
acggcggcgatcaacttcc

SEQ ID NO: 50 - DNA, 27 bp, artificial
  synthetic oligonucleotide primer
cgcgcggtacctgtcagtgccgtcaag

SEQ ID NO: 51 - DNA, 29 bp, artificial
  synthetic oligonucleotide primer
cgcgcccatggtttctgactaaagaatcg

SEQ ID NO: 52 - DNA, 1059 bp, Populus trichocarpa
aagcaatcactaccttcaactctcaaactctccacttctcaactcaaacctaccgaaatatattctttgcatgcagcttccaaaacacaaacaaacaatatccagtcccctttacttttccattccaaacccgaaaagtgcaaggacctttttttgagtttctctcgctctatcccaaagatcaaatccaatatgaggcctaaaggccaacaaatcaccaatcttaagatgctcctctttggtgtcttccttgctttctttgtgcttttcgtgttgcgatcaggcatgccgtcttcccaggaaagtccatctcccacctctcaaccaggagttttaaaccccatgaaagaagctagagaaacaaattgctcacaagcatgcactcagatcccacgctccctaactcaagctctcgttcactacacaacctcaaccatcaccccacagcagacacgcaaagaaatatcagtgagtgccaaagttttagaaaagaaatccccatgtaacttcttggtttttggcctcggtcatgatagccttatgtggagtgcactcaactacggtggacgaacagttttcttagaagaagatgaagcttggattgcgcaaattaaacgccggtttcccatgttagagtcccatcatgtcacatatgacagcaaggtgaatgaagcggccaatctcatggaagtaggcaagggaccggagtgcacagcagttggtgatccgaagttctccatgtgccaacttgccatgaaaggtttgcctagtgaagtttatgagatagaatgggacttgatcatggttgatgcaccaacaggctaccatgatgaggcaccagggagaatgactgccatatttacagctggaatgatggcacgaaacaggaaagagggagagaccgatgtttttgtgcacgatgtgaacagagaggtggaagataagttctccaagtcatttctttgtgaaggatatatgaagaaacaagttggaaggttaaggcacttcaccattcctagccacagggatgacttggacagacccttttgcccagagtag

SEQ ID NO: 53 - protein, 346 aa, artificial
  consensus sequence for GXMT polypeptides
KQSLPSTLKLSTSQLKPTEIYSLHAASKTQTNNIQSPLLFHSKPEMSSPMHARKAIHASLKAAMQKNLLLLLAALAFLLLFRTLSSSSSSLSSTKQPKPSTPACPSPCKLPSLAEALVHYTSSITPTSSRQQTEISVSRVLKKSPPCNFLVFGGLLGHDSLMWAALNHGQGGRTVFLEEDAWIAIRRHKPLESYHVYDTKVDADLMELRDHPECAVDLNTQLSPADPRSCLALKGLPLPPVPADFYELEWDLIMVDAPTGYHDEAPGRMGAIYTAGMMARRREGGPGDGTDVFVHDVDRVEDFSKAFLCEGYLEQVGRLRHFVIPSHRDKDESDSGPFCPDISRRR